(19) United States
(12) Patent Application Publication
(10) Pub. No.: US 2016/0207991 A1
(43) Pub. Date: Jul. 21, 2016

(54) Therapeutic Antibody

(71) Applicant: Pfizer Inc., New York, NY (US)

(72) Inventors: Laird Bloom, Needham, MA (US); Qingcong Lin, Needham, MA (US); Heather Hongrong Shih, Andover, MA (US); Ying Sun, Maynard, MA (US); Orla Margaret Cunningham, Clondalkin (IE); William James Jonathan Finlay, Clondalkin (IE)

(73) Assignee: PFIZER INC., New York, NY (US)

(21) Appl. No.: 14/842,422

(22) Filed: Sep. 1, 2015

Related U.S. Application Data
(60) Provisional application No. 62/044,579, filed on Sep. 2, 2014.

(51) Int. Cl. C07K 16/22; A61K 45/06; A61K 39/395; A61K 47/48; A61K 51/10

(52) U.S. Cl. CPC C07K 16/22; A61K 47/48546; A61K 51/1021; A61K 39/3955; A61K 45/06; C07K 2317/76; C07K 2317/56; C07K 2317/92; A61K 2039/505

(57) ABSTRACT

The present invention relates to antibodies that bind brain-derived neurotrophic factor (BDNF). The invention further relates to nucleic acid sequences coding for such antibodies. The present invention also relates to immunoconjugates comprising the antibodies of the invention and pharmaceutical compositions comprising the antibodies and/or the immunoconjugates. The present invention further relates to methods for treating pain and medical uses relating thereto.

This application claims the benefit under 35 U.S.C. §119(e) of U.S. Provisional Patent Application No. 62/044,579, filed on Sep. 2, 2014, the disclosure of which is hereby incorporated by reference in its entirety.

### REFERENCE TO SEQUENCE LISTING

This application is being filed electronically via EFS-Web and includes an electronically submitted sequence listing in .txt format. The .txt file contains a sequence listing entitled “PC72113_ST25.txt” created on Aug. 31, 2015 and having a size of 22 KB. The sequence listing contained in this .txt file is part of the specification and is herein incorporated by reference in its entirety.

### FIELD OF THE INVENTION

The present invention relates to antibodies that bind brain-derived neurotrophic factor (BDNF). The invention further relates to nucleic acid sequences coding for such antibodies. The present invention also relates to immunoconjugates comprising the antibodies of the invention and pharmaceutical compositions comprising the antibodies and/or the immunoconjugates. The present invention further relates to methods for treating pain and medical uses relating thereto.

### BACKGROUND OF THE INVENTION

Brain-derived neurotrophic factor BDNF, is a small soluble protein with molecular weight of 13 kDa for the monomer (27 kDa as homodimer) that belongs to the neurotrophin family of growth factors. It shares amino acid sequence homology to other family members including Nerve Growth Factor (NGF), Neurotrophin-3 (NT-3) and Neurotrophin-4 (NT-4) and is composed of a highly homologous structure containing antiparallel β strands and cysteine residues in a cystine knot motif. BDNF is important in developmental neurobiology where it controls aspects of survival, differentiation and proliferation of neurons in both the peripheral and central nervous systems. Furthermore, in adulthood, BDNF controls aspects of neuronal function, where it regulates synapse formation and synaptic plasticity.

Although widely expressed in a number of tissues, BDNF is highly abundant in the brain and its activity is linked to processes such as long term potentiation that underlies learning and memory. BDNF mutant (BDNF −/−) mice suffer developmental defects and usually fail to survive beyond the second postnatal week. Mice lacking BDNF display sensory neuron losses particularly in the vestibular and nododse-petrosal ganglion, that affect coordination and balance, suggesting that BDNF plays an important role in normal neural development. The physiological actions of BDNF are mediated via interaction with two types of receptors; the high affinity tyrosine receptor kinase B (TrkB) and p75NTR also known as low-affinity nerve growth factor receptor (LNGFR).

BDNF engagement of the TrkB receptor results in the dimerization of the TrkB receptor, leading to autophosphorylation of tyrosine residues in the cytoplasmic domain and enhanced tyrosine kinase activity of the receptor. This yields docking sites for adapter proteins containing phosphotyrosine-binding (PTB) or src-homology-2 (SH-2) motif that couple the receptor to multiple intracellular signaling cascades such as Ras/ERK (extracellular signal-regulated kinase), PI3K (phosphatidylinositol-3-kinase) and PLC-γ (phospholipase C γ). These pathways are involved in different aspects of neurone development and cell function including cell survival, differentiation, neurite outgrowth and synapse formation. The lower affinity p75NTR on the other hand, is a member of the tumour necrosis receptor superfamily. Unlike TrkB, it lacks intrinsic catalytic activity and contains a death domain in the cytoplasmic sequence. All members of the neurotrophin family activate p75NTR with similar affinities and ligand engagement leads to activation of several intracellular signal transduction pathways, including nuclear factor-κB (NF-κB), Jun kinase and sphingo-myelin hydrolysis. Trk-p75NTR interaction has been proposed to critically regulate Trk receptor signalling and furthermore enhance the ligand specificity of Trk receptors. The functional role of p75NTR is diverse and is implicated in both pro- and antitrophic processes, including neurite outgrowth and ligand mediated apoptosis.

Dysregulation in BDNF levels has been documented in a number of human disease conditions including joint disease, peripheral nerve damage, intervertebral disc degeneration and visceral conditions such as inflammatory bowel syndrome, chronic pancreatitis and overactive bladder. Correlations between peripheral BDNF levels and pain or disease severity have been documented. Accordingly, there is a need to provide agents that specifically and preferably selectively recognize and interact with BDNF and dampen or inhibit BDNF signalling through its receptor and to provide for therapeutic use of such agents particularly in conditions associated with BDNF, for example in chronic pain.

### SUMMARY OF THE INVENTION

The present invention provides isolated monoclonal antibodies, in particular chimeric and humanised monoclonal antibodies, or antigen-binding portions thereof, that bind specifically to BDNF, particularly human BDNF and exhibit numerous desirable properties including selectivity of binding to BDNF over other neurotrophins and inhibition of BDNF-mediated receptor binding and biological activity. Also provided are nucleic acids encoding such antibodies and vectors and cells comprising such nucleic acids as well as methods of producing such antibodies.

In particular the present invention relates to an isolated monoclonal antibody or an antigen-binding portion thereof that binds specifically to BDNF. More particularly, the isolated monoclonal antibody, or an antigen-binding portion thereof competes for binding to BDNF with and/or binds to the same epitope on BDNF as any of the anti-BDNF monoclonal antibodies of the invention as described herein.

The antibody or antigen binding portion thereof may compete for binding with and/or bind to the same epitope as a reference antibody comprising:

(i) a heavy chain variable region comprising the amino acid sequence of SEQ ID NO:14 and a light chain variable region comprising the amino acid sequence of SEQ ID NO:16; or
(ii) a heavy chain variable region comprising the amino acid sequence of SEQ ID NO:4 and a light chain variable region comprising the amino acid sequence of SEQ ID NO:6; or
(iii) a heavy chain variable region comprising the amino acid sequence of SEQ ID NO:18 and a light chain variable region comprising the amino acid sequence of SEQ ID NO:20; or
(iv) a heavy chain variable region comprising the amino acid sequence of SEQ ID NO:22 and a light chain variable region comprising the amino acid sequence of SEQ ID NO:24; or comprises:
(vii) a heavy chain region comprising the heavy chain variable region sequence encoded by the plasmid deposited at the ATCC and having ATCC Accession No. PTA-121203 and a light chain region comprising the light chain variable region sequence encoded by the plasmid deposited at the ATCC and having ATCC Accession No. PTA-121204, or
(viii) a heavy chain region comprising the heavy chain variable region sequence encoded by the plasmid deposited at the ATCC and having ATCC Accession No. PTA-121201 and a light chain region comprising the light chain variable region sequence encoded by the plasmid deposited at the ATCC and having ATCC Accession No. PTA-121202.

The isolated monoclonal antibody or an antigen-binding portion thereof of the invention may bind specifically to BDNF and may bind selectively to BDNF, optionally human BDNF. The isolated monoclonal antibody or an antigen-binding portion thereof of the invention may inhibit the interaction of BDNF with the receptor TrkB and/or p75NTR and may inhibit the biological activity of BDNF, in particular the biological activity of BDNF at the TrKB and/or p75NTR receptor. The invention also provides an isolated nucleic acid molecule encoding the antibody or antigen-binding portion thereof, optionally comprised within an expression vector. A host cell comprising the expression vector and methods for preparing the anti-BDNF antibody by expressing the antibody in the host cell are also provided.

The present invention additionally relates to an immunoconjugate comprising the antibody, or antigen-binding portion thereof, and to pharmaceutical compositions comprising the antibody or antigen-binding portion thereof, or the immunoconjugate, optionally further comprising a pharmaceutically acceptable carrier.

The antibody, or antigen-binding portion thereof, immunoconjugate or pharmaceutical composition is also provided for use in a method of treating or preventing a disease condition, particularly pain, which pain may be chronic or acute, more particularly pain selected from inflammatory pain, nociceptive pain, visceral pain and neuropathic pain, which pain may be chronic or acute. The present invention further provides the antibody, or antigen-binding portion thereof, the immunoconjugates or pharmaceutical compositions for use as a medicament and for use in treating or preventing pain, which pain may be chronic or acute, particularly inflammatory pain, nociceptive pain, visceral pain and neuropathic pain, which pain may be chronic or acute. Also provided are methods for treating a variety of diseases using the antibodies, antigen-binding portion thereof, immunoconjugates and pharmaceutical compositions of the invention, including the treatment or prevention of pain, which pain may be chronic or acute, particularly inflammatory pain, visceral pain, nociceptive pain and neuropathic pain which may be acute or chronic pain. The antibody, or antigen-binding portion thereof, immunoconjugate or pharmaceutical composition are also provided for use separately, sequentially or simultaneously in combination with a second therapeutic agent as a medicament for use in the foregoing treatments or methods of treatment.

### BRIEF DESCRIPTION OF THE DRAWINGS

FIG. 1: Provides an amino acid alignment of mouse, rat, human and chicken BDNF. Differences in sequence are marked with ‘.’ where one sequence varies or ‘:’ where two sequences in the series vary from the reference sequence (mouse BDNF).

FIG. 2: Crystal structure of BDNF-homodimer in complex with the neutralizing antibody fragment R3BH1-Fab.

FIG. 3: Detailed structure of epitope 1, involving the antibody heavy (A) and light (B) chains, represented by the molecular ribbons and the BDNF-cytokine chains of the homodimer (F and G).

FIG. 4: Anti-BDNF R3BH1 binding to BDNF measured by SPR on the BIAcore T200.

FIG. 5: Anti-BDNF R3BH1 displaces TrkB receptor bound BDNF in a competition HTRF assay.

FIG. 6: SPR anti-BDNF R3BH1 inhibition of BDNF binding to immobilised p75NTR.

FIG. 7: BDNF neurotrophin/chemokine interaction assay. All antibodies were titrated in a dilution series from 0-300 μg/mL. Only the highest concentration is shown here for clarity.

FIG. 8: Cell based ERK phosphorylation assay in U20S TrkB/p75NTR cells. Anti-BDNF antibody R3BH1 and TrkB-Fc molecule inhibits TrkB receptor activation and downstream signalling mediated by BDNF, as measured by phosphorylated pERK activity.

FIG. 9: Cell based TrkB phosphorylation assay in U20S TrkB/p75NTR cells. Anti-BDNF antibody R3BH1 and BDNF scavenging molecule, TrkB-Fc inhibits BDNF mediated TrkB receptor activation while the negative control had no effect.

FIG. 10: HTRF screening assay to identify affinity-optimized R3BH1 variants. Affinity optimised clones displace TrkB receptor bound BDNF in a competition HTRF assay.

FIG. 11: Anti-BDNF binding of humanised anti-BDNF clones to BDNF measured by SPR on the BIAcore T200.

FIG. 12: Crystal structure of BDNF-homodimer in complex with the neutralizing antibody fragment F30-Fab.

FIG. 13: BDNF neurotrophin/chemokine interaction assay. All antibodies were titrated in a dilution series from 0-300 μg/mL. Only the highest concentration is shown here for clarity.

FIG. 14: Cell based ERK phosphorylation assay in U20S TrkB/p75NTR cells. The humanised anti-BDNF molecule, B30 demonstrated greater BDNF binding compared to R3BH1 and TrkB-Fc, as measured by inhibition of pERK activity in U20S TrkB/p75NTR cells.

FIG. 15: Improved BDNF binding of humanised B30 clone in cell based TrkB phosphorylation assay in U20S TrkB/p75NTR cells. B30 clone inhibits BDNF mediated TrkB receptor phosphorylation in TrkB/p75NTR U2OS cells.

FIG. 16: Ligand binding assay using a fluorescent readout for total BDNF measured in plasma following intravenous dosing of rats with anti-BDNF antibody R3BH1 and humanised anti-BDNF molecule, B30.

FIG. 17: In vitro electrophysiology in dissociated dorsal root ganglion (DRG) neurones. Anti-BDNF antibody, R3BH1 reverses alterations in Kv current in a rat model of neuropathic pain. (A) Representative traces of Kv current recordings from uninjured (contralateral) and injured (ipsilateral) DRG neurons. Peripheral nerve injury causes downregulation of Kv channels and suppression of the Kv current. (B)/(C) Systemic administration of anti-BDNF antibody, R3BH1 reverses injury induced Kv suppression in a dose dependent manner. 10 mg/kg dose of R3BH1 fully reversed the Kv suppression seen in nerve injured animals.

FIG. 18: In vitro electrophysiology in dissociated DRG neurones. Humanised anti-BDNF antibody, B30 reverses alterations in Kv current induced by nerve injury in a rat model of neuropathic pain. (A)/(B) Systemic administration of anti-BDNF antibody, B30 reverses Kv suppression in a dose dependent manner. A dose of 0.1 mg/kg was shown to be effective in the model.

FIG. 19: Evaluation of the effects of the humanised anti-BDNF molecule, B30 on nerve injury induced thermal hypersensitivity in an ex vivo skin nerve preparation. Heat stimulation was delivered using a slow ramp (Ai) or fast ramp protocol (Aii). Animals treated with hIgG isotype control shows a sensitised heat response to slow ramp application. Anti-BDNF molecule, B30 dose dependently reduces the heat hypersensitivity seen in the injured leg. All data are presented as mean values±95% confidence intervals. *p<0.05, ***p<0.001.

FIG. 20: In vivo electrophysiological recordings of spinal dorsal horn neurones in rats sustaining peripheral nerve injury. Mechanical punctate (von Frey) responses were dose dependently attenuated by the anti-BDNF molecule, B30 (0.1 and 1 mg/kg) and pregabalin. Responses to heat stimuli were similarly attenuated by the anti-BDNF molecule, B30.

### DETAILED DESCRIPTION

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### DEFINITIONS

As used herein, the terms “brain derived neurotrophic factor” and “BDNF” refer to brain derived neurotrophic factor and variants thereof that retain at least part of the biological activity of BDNF. As used herein, BDNF includes all mammalian species of native sequence BDNF, including human, rat, mouse and chicken. The term “BDNF” is used to include variants, isoforms and species homologs of human BDNF. Antibodies of the invention may, in certain cases, cross-react with BDNF from species other than human. In certain embodiments, the antibodies may be completely specific for human BDNF and may not exhibit non-human cross-reactivity. The complete amino acid sequence of an exemplary human BDNF has Genbank accession number: CAA62632.1 (and is designated herein as SEQ ID NO:1).

As used herein, “p75NTR” is the p75 neurotrophic receptor and “trkB” is the tropomyosin-receptor-kinase B and are receptors for BDNF or are BDNF receptors, and include the TrkB receptor and the p75NTR receptor of any mammalian species, including, but are not limited to, human, rat, mouse and chicken.

As used herein, an “antagonist” as used in the context of the antibody of the invention or an “anti-BDNF antagonist antibody” (interchangeably termed “anti-BDNF antibody”) refers to an antibody which is able to bind to BDNF and inhibit BDNF biological activity and/or downstream pathway(s) mediated by BDNF signalling. An anti-BDNF antagonist antibody encompasses antibodies that can block, antagonize, suppress or reduce (including significantly) BDNF biological activity, including downstream pathways mediated by BDNF signalling, such as receptor binding and/or elicitation of a cellular response to BDNF. For the purposes of the present invention, it will be explicitly understood that the term “anti-BDNF antagonist antibody” encompass all the herein identified terms, titles, and functional states and characteristics whereby BDNF itself, and BDNF biological activity (including but not limited to its ability to mediate any aspect of pain), or the consequences of the activity or biological activity, are substantially nullified, decreased, or neutralized in any meaningful degree. In some embodiments, an anti-BDNF antibody or anti-BDNF antagonist antibody binds BDNF and prevents BDNF induced p75NTR and/or trkB receptor dimerisation and/or autophosphorylation and/or binding to a BDNF receptor (such as p75NTR and/or trkB). Examples of anti-BDNF antibodies or anti-BDNF antagonist antibodies are provided herein.

“Biological activity”, “BDNF activity” or “activity” in the context of BDNF generally refers to the ability to bind BDNF receptors (trkB and/or p75NTR) and/or activate BDNF receptor signalling pathways. Without limitation, a biological activity includes any one or more of the following: the ability to bind a BDNF receptor (such as p75NTR and/or trkB); the ability to promote trkB and/or p75NTR receptor dimerization and/or autophosphorylation; the ability to activate a BDNF receptor signalling pathway; the ability to promote or effect cell or neuron biology such as for example, cell differentiation, proliferation, survival, growth and other changes in cell physiology, including (in the case of neurons, including peripheral and central neurons) change in neuronal morphology, synaptogenesis, synaptic function, neurotransmitter and/or neuropeptide release and regeneration following damage; the ability to promote differentiation and proliferation of neurons in both the peripheral and central nervous systems, control of aspects of neuronal function and regulation of synapse formation and synaptic plasticity and neural development and/or the ability to mediate pain for example chronic or acute pain, particularly inflammatory pain, nociceptive pain, visceral pain or neuropathic pain, which pain may be chronic or acute, more particularly neuropathic and/or inflammatory pain, and/or chronic pain.

BDNF “specifically binds” “specifically interacts”, “preferentially binds”, “binds” or “interacts” with a receptor such as trkB or p75NTR if it binds with greater affinity, avidity, more readily, and/or with greater duration than it binds to other receptors, particularly other neurotrophin receptors. “Specifically binds” “specifically interacts” or “preferentially binds” in the context of BDNF binding to a BDNF receptor generally refers to the ability to bind BDNF receptors (trkB and/or p75NTR) and/or the ability to promote trkB and/or p75NTR receptor dimerization and/or autophosphorylation and/or activate a BDNF receptor signalling pathway.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also any antigen binding fragment (i.e., “antigen-binding portion”) or single chain thereof, fusion proteins comprising an antibody, and any other modified configuration of the immunoglobulin molecule that comprises an antigen recognition site including, for example without limitation, scFv, single domain antibodies (e.g., shark and camelid antibodies), maxibodies, minibodies, intrabodies, diabodies, triabodies, tetrabodies, v-NAR and bis-scFv (see, e.g., Hollinger and Hudson, 2005, Nature Biotechnology 23(9): 1126-1136). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody, as used herein, refers to one or more fragments of an intact antibody that retain the ability to specifically bind to BDNF. Antigen binding functions of an antibody can be performed by fragments of an intact antibody. Examples of binding fragments encompassed within the term “antigen binding portion” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., 1989 Nature 341:544-546), and an isolated complementarity determining region (CDR).

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987). When choosing FR to flank subject CDRs, e.g., when humanizing or optimizing an antibody, FRs from antibodies which contain CDR1 and CDR2 sequences in the same canonical class are preferred.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1989, Nature 342:877-883. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., 1996, J. Mol. Biol., 262:732-745. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

The term “monoclonal antibody” (Mab) refers to an antibody, or antigen-binding portion thereof, that is derived from a single copy or clone, including e.g., any eukaryotic, prokaryotic, or phage clone, and not the method by which it is produced. Preferably, a monoclonal antibody of the invention exists in a homogeneous or substantially homogeneous population.

“Humanized” antibody refers to forms of non-human (e.g. murine or chicken) antibodies, or antigen-binding portion thereof, that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity.

“Human antibody or fully human antibody” refers to those antibodies, or antigen-binding portion thereof, derived from transgenic mice carrying human antibody genes or from human cells.

The term “chimeric antibody” is intended to refer to antibodies, or antigen-binding portion thereof, in which the variable region sequences are derived from one species and the constant region sequences are derived from another species, such as an antibody in which the variable region sequences are derived from a mouse antibody and the constant region sequences are derived from a human antibody.

“Antibody-drug conjugate” and “immunoconjugate” refer to antibodies, or antigen-binding portion thereof, including antibody derivatives that bind to BDNF and are conjugated to cytotoxic, cytostatic, and/or therapeutic agents.

Antibodies of the invention, or antigen-binding portion thereof, can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50 (1999) and Fellouse, F. A., et al, J. Mol. Biol., 373(4):924-40 (2007)).

The term “epitope” refers to that portion of a molecule capable of being recognized by and bound by an antibody, or antigen-binding portion thereof, at one or more of the antibody's antigen-binding regions. Epitopes can consist of defined regions of primary secondary or tertiary protein structure and includes combinations of secondary structural units or structural domains of the target recognised by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody can specifically bind as determined by any method well known in the art, for example, by conventional immunoassays, antibody competitive binding assays or by x-ray crystallography or related structural determination methods (for example NMR). A “nonlinear epitope” or “conformational epitope” comprises noncontiguous polypeptides (or amino acids) within the antigenic protein to which an antibody specific to the epitope binds. Once a desired epitope on an antigen is determined, it is possible to generate antibodies to that epitope, e.g., using the techniques described in the present specification. During the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another e.g., the antibodies compete for binding to the antigen or antigenic epitope.

An epitope that “specifically binds”, “specifically interacts” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to BDNF or a BDNF epitope is an antibody that binds BDNF or the BDNF epitope with greater affinity, avidity, more readily, and/or with greater duration than it binds to other neurotrophins or chemokines or to other BDNF epitopes or non-BDNF epitopes, for example it is also selective for BDNF over other neurotrophins or chemokines. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

Binding selectivity in the context of antibody ligand interaction is a relative or comparative term indicating that the antibody can bind with differing affinities with different ligands such as neurotrophins or chemokines to form a complex. Where an antibody is described as selectively binding BDNF or human BDNF this indicates that in comparison to binding other neurotrophins or chemokines the equilibrium constant for the reaction of displacement of BDNF from the binding site of the antibody lies in the direction of the BDNF-antibody complex in comparison to the antibody complex with the other or related neurotrophins or chemokines.

The term “binding affinity” or “K_{D}” as used herein, is intended to refer to the dissociation rate of a particular antigen-antibody interaction. The K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M). It follows that the smaller the K_{D}, the stronger the affinity of binding. Therefore, a K_{D} of 1 μM indicates weak binding affinity compared to a K_{D} of 1 nM. K_{D} values for antibodies can be determined using methods well established in the art. One method for determining the K_{D} of an antibody is by using surface plasmon resonance (SPR), typically using a biosensor system such as a Biacore® system.

The term “potency” is a measurement of biological activity and may be designated as IC₅₀, or effective concentration of an antibody or antibody drug conjugate to the antigen BDNF to inhibit 50% of activity measured in a BDNF activity assay such as the pERK or Pathfinder assay described herein.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The term “inhibit” or “neutralize” as used herein with respect to bioactivity of an antibody of the invention means the ability of the antibody to substantially antagonize, prohibit, prevent, restrain, slow, disrupt, eliminate, stop, reduce or reverse e.g. progression or severity of that which is being inhibited including, but not limited to, a biological activity or binding interaction between BDNF and p75NTR and/or trkB.

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment. For the avoidance of doubt, references herein to “treatment” also include references to curative, palliative and prophylactic treatment.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples.

As used herein, “substantially pure” refers to material which is at least 50% pure (i.e., free from contaminants), more preferably at least 90% pure, more preferably at least 95% pure, more preferably at least 98% pure, more preferably at least 99% pure.

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

### Anti-BDNF Antibodies

According to a first aspect of the present invention there is provided an isolated anti-BDNF antibody, or an antigen-binding portion thereof, wherein the antibody:

(a) binds to human BDNF and
(b) competes for binding to human BDNF with and/or binds to the same epitope on human BDNF as, a reference antibody comprising:
(i) a heavy chain variable region comprising the amino acid sequence of SEQ ID NO:14 and a light chain variable region comprising the amino acid sequence of SEQ ID NO:16; or
(ii) a heavy chain variable region comprising the amino acid sequence of SEQ ID NO:4 and a light chain variable region comprising the amino acid sequence of SEQ ID NO:6; or
(iii) a heavy chain variable region comprising the amino acid sequence of SEQ ID NO:18 and a light chain variable region comprising the amino acid sequence of SEQ ID NO:20; or
(iv) a heavy chain variable region comprising the amino acid sequence of SEQ ID NO:22 and a light chain variable region comprising the amino acid sequence of SEQ ID NO:24; or

In an embodiment, the antibody or antigen-binding portion thereof, competes for binding to human BDNF with and/or binds to the same epitope on human BDNF as a reference antibody comprising; (i) a heavy chain region comprising the heavy chain variable region sequence encoded by the plasmid deposited at the ATCC and having ATCC Accession No. PTA-121201 and a light chain region comprising the light chain variable region sequence encoded by the plasmid deposited at the ATCC and having ATCC Accession No. PTA-121202, or

(i) a heavy chain region comprising the heavy chain variable region sequence encoded by the plasmid deposited at the ATCC and having ATCC Accession No. PTA-121203 and a light chain region comprising the light chain variable region sequence encoded by the plasmid deposited at the ATCC and having ATCC Accession No. PTA-121204.

The invention provides antibodies that compete for binding to human BDNF with and/or binds to the same epitope on human BDNF as any one or more of the anti-BDNF monoclonal antibodies of the invention. The invention therefore includes antibodies that have the ability to compete for binding to or cross-compete for binding to BDNF with any of the monoclonal antibodies of the invention. In an embodiment of the invention, the reference antibody for cross-competition studies can be the monoclonal antibody R3BH1 (having V_{H} and V_{L} sequences as shown in SEQ ID NOs: 4 and 6, respectively), or the monoclonal antibody B30 (having V_{H} and V_{L} sequences as shown in SEQ ID NOs: 14 and 16, respectively), or the monoclonal antibody B20 (having V_{H} and V_{L} sequences as shown in SEQ ID NOs: 18 and 20, respectively), or the monoclonal antibody B18 (having V_{H} and V_{L} sequences as shown in SEQ ID NOs: 22 and 24, respectively). Such cross-competing antibodies can be identified based on their ability to cross-compete with any one or more of R3BH1, B30, B20 or B18 in a BDNF binding assay. For example, BIAcore analysis, ELISA assays or flow cytometry may be used to demonstrate cross-competition with the antibodies of the current invention. For example, BDNF competition binding assays can be conducted using an ELISA format with plate bound BDNF in the presence of any of the reference antibodies R3BH1, B30, B20 or B18, which may for example be biotinylated, the effect of the test antibody on the binding of the reference antibody to BDNF can be readily determined. Antibodies can be biotinylated using commercially available reagents (Pierce, Rockford, Ill.). The ability of a test antibody to inhibit the binding of, for example, any one or more of R3BH1, B30, B20 or B18, to human BDNF demonstrates that the test antibody can compete with any one or more of R3BH1, B30, B20 or B18 for binding to human BDNF and/or binds to the same epitope on human BDNF as any one or more of R3BH1, B30, B20 or B18. In an embodiment of the present invention, the antibody that competes for binding to human BDNF with and/or binds to the same epitope on human BDNF as any one or more of R3BH1, B30, B20 or B18 is a chimeric, human or humanised monoclonal antibody. Such chimeric, human or humanised monoclonal antibodies can be prepared and isolated according to known methods. Methods of determining whether any particular anti-BDNF monoclonal antibody (test antibody) competes for binding to human BDNF with and/or binds to the same epitope as any one of the reference antibodies are known.

According to an embodiment of the invention there is provided an antibody, or antigen-binding portion thereof, which competes for binding to human BDNF with and/or binds to the same epitope on human BDNF as, a reference antibody comprising:

(i) a heavy chain variable region comprising CDR1, CDR2, CDR3 from SEQ ID NO: 14 and a light chain variable region comprising CDR1, CDR2, CDR3 from SEQ ID NO: 16, or
(ii) a heavy chain variable region comprising CDR1, CDR2, CDR3 from SEQ ID NO: 4 and a light chain variable region comprising CDR1, CDR2, CDR3 from SEQ ID NO: 6, or
(iii) a heavy chain variable region comprising CDR1, CDR2, CDR3 from SEQ ID NO: 18 and a light chain variable region comprising CDR1, CDR2, CDR3 from SEQ ID NO: 20, or
(iv) a heavy chain variable region comprising CDR1, CDR2, CDR3 from SEQ ID NO: 22 and a light chain variable region comprising CDR1, CDR2, CDR3 from SEQ ID NO: 24.

According to an embodiment of the invention there is provided an antibody, or antigen-binding portion thereof, which competes for binding to human BDNF with and/or binds to the same epitope on human BDNF as, a reference antibody comprising:
i. a heavy chain variable region CDR1 comprising SEQ ID NO: 25,
   a heavy chain variable region CDR2 comprising SEQ ID NO: 26,
   a heavy chain variable region CDR3 comprising SEQ ID NO: 27,
   a light chain variable region CDR1 comprising SEQ ID NO: 28,
   a light chain variable region CDR2 comprising SEQ ID NO: 29 and
   a light chain variable region CDR3 comprising SEQ ID NO: 30; or
ii. a heavy chain variable region CDR1 comprising SEQ ID NO: 7,
   a heavy chain variable region CDR2 comprising SEQ ID NO: 8,
   a heavy chain variable region CDR3 comprising SEQ ID NO: 9,
   a light chain variable region CDR1 comprising SEQ ID NO: 10,
   a light chain variable region CDR2 comprising SEQ ID NO: 11 and
   a light chain variable region CDR3 comprising SEQ ID NO: 12; or
iii. a heavy chain variable region CDR1 comprising SEQ ID NO: 31,
   a heavy chain variable region CDR2 comprising SEQ ID NO: 32,
   a heavy chain variable region CDR3 comprising SEQ ID NO: 33,
   a light chain variable region CDR1 comprising SEQ ID NO: 34,
   a light chain variable region CDR2 comprising SEQ ID NO: 35 and
   a light chain variable region CDR3 comprising SEQ ID NO: 36; or
iv. a heavy chain variable region CDR1 comprising SEQ ID NO: 37,
   a heavy chain variable region CDR2 comprising SEQ ID NO: 38,
   a heavy chain variable region CDR3 comprising SEQ ID NO: 39,
   a light chain variable region CDR1 comprising SEQ ID NO: 40,
   a light chain variable region CDR2 comprising SEQ ID NO: 41 and
   a light chain variable region CDR3 comprising SEQ ID NO: 42.

According to an embodiment of the invention there is provided an antibody, or antigen-binding portion thereof, which competes for binding to human BDNF with and/or binds to the same epitope on human BDNF as, as the reference antibody. In some embodiments the isolated monoclonal antibody, or an antigen-binding portion thereof, competes for binding for and/or binds to an epitope of human BDNF comprising residues within the region of ILE 16 to PHE 102, ILE 16 to Arg 104 or residues ILE 16 to ASN 106 of SEQ ID NO:1, or comprising residues ILE 16 to PHE 102, ILE 16 to Arg 104 or residues ILE 16 to ASN 106 of SEQ ID NO:1.

According to an embodiment of the invention, the isolated monoclonal antibody, or an antigen-binding portion thereof, competes for binding for and/or binds to, an epitope of human BDNF comprising a region comprised within both BDNF monomers in the BDNF homodimer, such as for example to a region comprising loop 1 and loop 4 of a first BDNF monomer and loop 2, loop 3 and the N-terminal region of a second BDNF monomer in the BDNF homodimer.

According to an embodiment of the invention, the isolated monoclonal antibody, or an antigen-binding portion thereof, competes for binding for and/or binds to, an epitope of human BDNF comprising:

(a) residues ILE 16, SER 17 TRP 19, THR 21, ALA 23, MET 31, SER 32, GLU 40, LYS 41, LYS 46, LEU 49, LYS 50, TYR 52, MET 61, ARG 88, LYS 95, ARG 97, GLY 99, TRP 100, ARG 101, PHE 102 of SEQ ID NO:1, or
(b) residues ILE 16, SER 17, TRP 19, THR 21, ALA 23, MET 31, SER 32, GLY 33, GLU 40, LYS 41, VAL 44, SER 45, GLN 48, LEU 49, LYS 50, TYR 52, TYR 86, TRP 100, ARG 101, PHE 102, ARG 104 of SEQ ID NO:1, or
(c) residues ILE 16, SER 17 TRP 19, ALA 23, MET 31, SER 32, GLU 40, LYS 41, LEU 49, LYS 50, TYR 52, MET 61, ARG 88, ARG 97, GLY 99, TRP 100, ARG 101, PHE 102 of SEQ ID NO:1, or
(d) residues ILEU 16, SER 17 TRP 19, THR 21, ALA 23, MET 31, SER 32, GLU 40, LYS 41, LYS 50, TYR 52, TRP 100, ARG 101, PHE 102 of SEQ ID NO:1
(e) residues TRP 19, LYS 41, LYS 50, TYR 52, ARG 88, ARG 97, ARG 101 of SEQ ID NO:1, or.
(f) residues ILE 16, MET 31, LEU 49, GLY 99, PHE 102 of SEQ ID NO:1, or
(g) residues, THR 21, SER 32, SER 17, GLU 40, MET 61, ASP 30 of SEQ ID NO:1, or residues ALA 23, GLN 48, TRP 100 of SEQ ID NO:1, or residues ILEU 98, GLU 18, ASP 24, ARG 104 of SEQ ID NO:1, or residues THR 21, LYS 46, LYS 95, of SEQ ID NO:1.

According to an embodiment of the invention, the isolated monoclonal antibody, or an antigen-binding portion thereof, competes for binding with the reference antibody for, and/or binds to, an epitope of human BDNF comprising a region comprising the residues MET 31, SER 32, ARG 88, LYS 95, ARG 97, GLY 99, TRP 100, ARG 101 and PHE 102, of SEQ ID NO:1, of a first BDNF monomer and residues ILE 16, SER 17 TRP 19, THR 21, ALA 23, GLU 40, LYS 41, LYS 46, LEU 49, LYS 50, TYR 52 and MET 61, of SEQ ID NO:1, of a second BDNF monomer of the homodimer.

According to an embodiment of the invention, the isolated monoclonal antibody, or an antigen-binding portion thereof, competes for binding with the reference antibody for, and/or binds to, an epitope of human BDNF comprising a region comprising the residues MET 31, SER 32, GLY 33, TYR 86, TRP 100, ARG 101, PHE 102 and ARG 104, of SEQ ID NO:1, of a first BDNF monomer and a residues ILE 16, SER 17, TRP 19, THR 21, ALA 23, GLU 40, LYS 41, VAL 44, SER 45, GLN 48, LEU 49, LYS 50 and TYR 52, of SEQ ID NO:1, of a second BDNF monomer of the homodimer.

According to an embodiment of the invention, the isolated monoclonal antibody, or an antigen-binding portion thereof, competes for binding with the reference antibody for, and/or binds to, an epitope of human BDNF comprising a region comprising the residues MET 31, SER 32, ARG 88, ARG 97, GLY 99, TRP 100, ARG 101 and PHE 102, of SEQ ID NO:1, of a first BDNF monomer and residues ILE 16, SER 17, TRP 19, ALA 23, GLU 40, LYS 41, LEU 49, LYS 50, TYR 52 and MET 61, of SEQ ID NO:1, of a second BDNF monomer of the homodimer.

According to an embodiment of the invention, the isolated monoclonal antibody, or an antigen-binding portion thereof, competes for binding with the reference antibody for, and/or binds to, an epitope of human BDNF comprising a region comprising the residues MET 31, SER 32, TRP 100, ARG 101 and PHE 102, of SEQ ID NO:1, of a first BDNF monomer and residues ILEU 16, SER 17 TRP 19, THR 21, ALA 23, GLU 40, LYS 41, LYS 50 and TYR 52, of SEQ ID NO:1, of a second BDNF monomer of the homodimer.

According to an embodiment of the invention, the isolated monoclonal antibody, or an antigen-binding portion thereof, competes for binding with the reference antibody for, and/or binds to, an epitope of human BDNF comprising a region comprising:

(i) residues ILEU 16, SER 17 TRP 19, THR 21, ALA 23, GLU 40, LYS 41, LYS 50 and TYR 52, of SEQ ID NO:1, of a first BDNF monomer and residues MET 31, SER 32, TRP 100, ARG 101 and PHE 102 of SEQ ID NO:1, of a second BDNF monomer of the homodimer, or
(ii) residues TRP 19, LYS 41, LYS 50, TYR 52, of SEQ ID NO:1, of a first BDNF monomer and residues ARG 88, ARG 97, ARG 101 of SEQ ID NO:1, of a second BDNF monomer of the homodimer, or
(iii) residues ILE 16, LEU 49, of SEQ ID NO:1, of a first BDNF monomer and residues MET 31, GLY 99, PHE 102 of SEQ ID NO:1, of a second BDNF monomer of the homodimer, or
(iv) residues, SER 17, GLU 40, MET 61, THR 21, of SEQ ID NO:1, of a first BDNF monomer and residues, ASP 30 SER 32, of SEQ ID NO:1, or
(v) or residues ALA 23, GLN 48, of SEQ ID NO:1, of a first BDNF monomer and residues TRP 100 of SEQ ID NO:1, of a second BDNF monomer of the homodimer, or
(vi) or residues GLU 18, ASP 24, of SEQ ID NO:1, of a first BDNF monomer and residues ILEU 98, ARG 104 of SEQ ID NO:1, of a second BDNF monomer of the homodimer, or
(vii) or residues THR 21, LYS 46, of SEQ ID NO:1, of a first BDNF monomer and residues LYS 95, of SEQ ID NO:1, of a second BDNF monomer of the homodimer.

According to an embodiment of the invention, two of the isolated monoclonal antibodies, or an antigen-binding portions thereof, of the invention bind together and/or simultaneously to the same BDNF homodimer, for example such that a pair of matched or identical epitopes as herein-before described are simultaneously bound on the same human BDNF homodimer.

According to an embodiment of the invention, the isolated monoclonal antibody, or an antigen-binding portion thereof, competes for binding with the reference antibody for, or binds to, a pair of matched or identical epitopes as herein-before described on the same human BDNF homodimer.

According to an embodiment of the invention, the antibody, or antigen-binding portion thereof, comprises:

(i) a heavy chain variable region comprising CDR1, CDR2, CDR3 of the heavy chain variable region sequence of SEQ ID NO: 14 and a light chain variable region comprising CDR1, CDR2, CDR3 of the light chain variable region sequence of SEQ ID NO: 16, or
(ii) a heavy chain variable region comprising CDR1, CDR2, CDR3 of the heavy chain variable region sequence of SEQ ID NO: 4 and a light chain variable region comprising CDR1, CDR2, CDR3 of the light chain variable region sequence of SEQ ID NO: 6, or
(iii) a heavy chain variable region comprising CDR1, CDR2, CDR3 of the heavy chain variable region sequence of SEQ ID NO: 18 and a light chain variable region comprising CDR1, CDR2, CDR3 of the light chain variable region sequence of SEQ ID NO: 20, or
(iv) a heavy chain variable region comprising CDR1, CDR2, CDR3 of the heavy chain variable region sequence of SEQ ID NO: 22 and a light chain variable region comprising CDR1, CDR2, CDR3 of the light chain variable region sequence of SEQ ID NO: 24.

According to an embodiment of the invention, the antibody, or antigen-binding portion thereof, comprises:

(i) a heavy chain variable region comprising
a heavy chain variable region CDR1 comprising SEQ ID NO: 25,
a heavy chain variable region CDR2 comprising SEQ ID NO: 26,
a heavy chain variable region CDR3 comprising SEQ ID NO: 27, and
a light chain variable region comprising
a light chain variable region CDR1 comprising SEQ ID NO: 28,
a light chain variable region CDR2 comprising SEQ ID NO: 29 and
a light chain variable region CDR3 comprising SEQ ID NO: 30; or
(ii) a heavy chain variable region comprising;
a heavy chain variable region CDR1 comprising SEQ ID NO: 7,
a heavy chain variable region CDR2 comprising SEQ ID NO: 8,
a heavy chain variable region CDR3 comprising SEQ ID NO: 9, and
a light chain variable region comprising
a light chain variable region CDR1 comprising SEQ ID NO: 10,
a light chain variable region CDR2 comprising SEQ ID NO: 11 and
a light chain variable region CDR3 comprising SEQ ID NO: 12; or
(iii) a heavy chain variable region comprising
a heavy chain variable region CDR1 comprising SEQ ID NO: 31,
a heavy chain variable region CDR2 comprising SEQ ID NO: 32,
a heavy chain variable region CDR3 comprising SEQ ID NO: 33, and
a light chain variable region comprising
a light chain variable region CDR1 comprising SEQ ID NO: 34,
a light chain variable region CDR2 comprising SEQ ID NO: 35 and
a light chain variable region CDR3 comprising SEQ ID NO: 36; or
(iv) a heavy chain variable region comprising
a heavy chain variable region CDR1 comprising SEQ ID NO: 37,
a heavy chain variable region CDR2 comprising SEQ ID NO: 38,
a heavy chain variable region CDR3 comprising SEQ ID NO: 39, and
a light chain variable region comprising
a light chain variable region CDR1 comprising SEQ ID NO: 40,
a light chain variable region CDR2 comprising SEQ ID NO: 41 and
a light chain variable region CDR3 comprising SEQ ID NO: 42.

According to an embodiment of the invention, the antibody, or antigen-binding portion thereof, comprises:

(i) a heavy chain region comprising the heavy chain variable region sequence of SEQ ID NO: 14 and a light chain region comprising the light chain variable region sequence of SEQ ID NO: 16, or
(ii) a heavy chain region comprising the heavy chain variable region sequence of SEQ ID NO: 4 and a light chain region comprising the light chain variable region sequence of SEQ ID NO: 6, or
(iii) a heavy chain region comprising the heavy chain variable region sequence of SEQ ID NO: 18 and a light chain region comprising the light chain variable region sequence of SEQ ID NO: 20, or
(iv) a heavy chain region comprising the heavy chain variable region sequence of SEQ ID NO: 22 and a light chain region comprising the light chain variable region sequence of SEQ ID NO: 24.

According to an embodiment of the invention, the antibody, or antigen-binding portion thereof, comprises:

(i) a heavy chain region comprising the heavy chain variable region sequence encoded by the plasmid deposited at the ATCC and having ATCC Accession No. PTA-121201 and a light chain region comprising the light chain variable region sequence encoded by the plasmid deposited at the ATCC and having ATCC Accession No. PTA-121202, or
(ii) a heavy chain region comprising the heavy chain variable region sequence encoded by the plasmid deposited at the ATCC and having ATCC Accession No. PTA-121203 and a light chain region comprising the light chain variable region sequence encoded by the plasmid deposited at the ATCC and having ATCC Accession No. PTA-121204.

Particular embodiments of the antibody of the invention are the chimeric chicken antibody R3BH1 and the humanised antibodies B30, B20 and B18, the VH, VL amino acid sequences, VH/VL nucleotide sequences and CDR amino acid sequences of these antibodies are provided in Tables 1 to 3 respectively.

TABLE 1
Anti BDNF antibody VH and VL amino acid sequences and antigen
binding CDR sequences according to Kabat (underlined).

| MAb | Sequence | Region | SEQ ID NO. |
|---|---|---|---|
| R3B | AVTLDESGGGLQTPGGGLSLVCKASGFDFSSYDMHWVRQAPGKGL | VH | SEQ ID NO: 4 |
| H1 | EWVAGIDDGGSDTYYGSAVKGRATISRDNGQSTVRLQLNNLRAED | | |
| | TGTYYCAKSSYDISWNGHVENIDAWGHGTEVIVSS | | |
| R3B | ALTQPTSVSTNLGGTVEITCSGAGSGYGYGWFQQKSPGSAPVTVI | VL | SEQ ID NO: 6 |
| H1 | YSNDKRPSDIPSRFSGSKSGSTGTLTITGVQAEDEAVYFCGTYDS | | |
| | TDAGYAIFGAGTTLTVL | | |
| B30 | EVQLLESGGGLVQPGGSLRLSCAASGFDFSSYDMHWVRQAPGKGL | VH | SEQ ID NO: 14 |
| | EWVSGIGDYGIETYYGSAVKGRFTISRDNSKNTLYLQMNSLRAED | | |
| | TAVYYCAKSSYDISWNGHVEHIDSWGQGTLVTVSS | | |
| B30 | SSELTQPPAVSVALGQTVRITCSGAGSGYGYGWYQQKPGQAPVTV | VL | SEQ ID NO: 16 |
| | IYSNDKRPSGIPDRFSGSSSGNTASLTITGAQAEDEADYYCGTYV | | |
| | SAYYGYAIFGGGTKLTVL | | |
| B20 | EVQLLESGGGLVQPGGSLRLSCAASGFDFSSYDMHWVRQAPGKGL | VH | SEQ ID NO: 18 |
| | EWVSGIDDYGIETYYGSAVKGRFTISRDNSKNTLYLQMNSLRAED | | |
| | TAVYYCAKSSYDISWNGHVEHLDAWGQGTLVTVSS | | |
| B20 | SSELTQPPAVSVALGQTVRITCSGAGSGYGYGWYQQKPGQAPVTV | VL | SEQ ID NO: 20 |
| | IYSNDKRPSGIPDRFSGSSSGNTASLTITGAQAEDEADYYCGTYD | | |
| | STDAGYAIFGGGTKLTVL | | |
| B18 | EVQLLESGGGLVQPGGSLRLSCAASGFDFSSYDMHWVRQAPGKGL | VH | SEQ ID NO: 22 |
| | EWVSGIDDYGIETYYGSAVKGRFTISRDNSKNTLYLQMNSLRAED | | |
| | TAVYYCAKSSYDISWNGHVEHLDAWGQGTLVTVSS | | |
| B18 | SSELTQPPAVSVALGQTVRITCQGDSSGYGYGWYQQKPGQAPVTV | VL | SEQ ID NO: 24 |
| | IYGKNNRPSGIPDRFSGSSSGNTASLTITGAQAEDEADYYCGTYV | | |
| | SAYYGYAIFGGGTKLTVL | | |

TABLE 2
Anti BDNF antibody VH and VL nucleotide sequences

| MAb | Sequence | Region | SEQ ID NO. |
|---|---|---|---|
| R3B | GCCGTGACGTTGGACGAGTCCGGGGGCGGCCTCCAGACGCCCG | VH | SEQ ID NO: 3 |
| H1 | GAGGAGGGCTCAGCCTCGTCTGCAAGGCCTCCGGGTTCGACTT | nucleotide | |
| | CAGCAGTTACGACATGCACTGGGTGCGACAGGCGCCCGGCAAA | | |
| | GGGCTGGAATGGGTCGCTGGTATTGATGATGGCGGTAGTGACA | | |
| | CATACTACGGGTCGGCGGTGAAGGGCCGTGCCACCATCTCGAG | | |
| | GGACAACGGGCAGAGCACAGTGAGGCTGCAGCTGAACAACCTC | | |
| | AGGGCTGAGGACACCGGCACCTACTACTGCGCCAAAAGCAGTT | | |
| | ATGACATTAGTTGGAATGGTCATGTTGAAAATATCGACGCATG | | |
| | GGGCCACGGGACCGAAGTCATCGTCTCCTCT | | |
| R3B | GCCCTGACTCAGCCGACCTCGGTGTCAACAAACCTGGGAGGAA | VL | SEQ ID NO: 5 |
| H1 | CCGTCGAGATCACCTGCTCCGGGGCTGGAAGTGGCTATGGTTA | nucleotide | |
| | TGGCTGGTTCCAGCAGAAGTCTCCTGGCAGTGCCCCTGTCACT | | |
| | GTGATCTATAGCAACGACAAGAGACCCTCGGACATCCCTTCAC | | |
| | GATTCTCCGGTTCTAAATCCGGCTCCACGGGCACATTAACCAT | | |
| | CACTGGGGTCCAAGCCGAGGACGAGGCTGTCTATTTCTGTGGG | | |
| | ACCTACGACAGCACTGATGCTGGTTATGCTATATTTGGGGCCG | | |
| | GGACAACCCTGACCGTCCTA | | |
| B30 | GAGGTGCAGCTGTTGGAGTCTGGGGGAGGCTTGGTGCAGCCTG | VH | SEQ ID NO: 13 |
| | GGGGGTCCCTGAGACTCTCCTGTGCAGCCTCTGGGTTCGACTT | nucleotide | |
| | CAGCAGTTACGACATGCACTGGGTCCGCCAGGCTCCAGGGAAG | | |
| | GGGCTGGAGTGGGTCTCAGGTATTGGTGATTACGGTATTGAAA | | |
| | CATACTACGGGTCCGCTGTGAAGGGCCGGTTCACCATCTCCAG | | |
| | AGACAATTCCAAGAACACACTGTATCTGCAAATGAACAGCCTG | | |
| | AGAGCCGAGGACACCGCCGTGTATTACTGTGCCAAAAGCAGTT | | |
| | ATGACATTAGTTGGAATGGTCATGTTGAACATATCGACTCATG | | |
| | GGGCCAGGGGACCCTGGTCACCGTCTCCTCT | | |
| B30 | TCTTCTGAGCTGACTCAGCCTCCTGCTGTGTCTGTGGCCTTGG | VL | SEQ ID NO: 15 |
| | GACAGACAGTCAGGATCACATGCTCCGGGGCTGGAAGTGGCTA | nucleotide | |
| | TGGTTATGGCTGGTACCAGCAGAAGCCAGGACAGGCCCCTGTG | | |
| | ACCGTCATCTATAGCAACGACAAGAGACCCTCCGGGATCCCAG | | |
| | ACCGATTCTCTGGCTCCAGCTCAGGAAACACAGCTTCCTTGAC | | |
| | CATCACTGGGGCTCAGGCCGAAGATGAGGCTGACTATTACTGT | | |
| | GGGACCTACGTCAGCGCATATTATGGTTATGCTATATTTGGGG | | |
| | GCGGGACAAAGCTGACCGTCCTA | | |
| B20 | GAGGTGCAGCTGTTGGAGTCTGGGGGAGGCTTGGTGCAGCCTG | VH | SEQ ID NO: 17 |
| | GGGGGTCCCTGAGACTCTCCTGTGCAGCCTCTGGGTTCGACTT | nucleotide | |
| | CAGCAGTTACGACATGCACTGGGTCCGCCAGGCTCCAGGGAAG | | |
| | GGGCTGGAGTGGGTCTCAGGTATTGATGATTACGGAATTGAAA | | |
| | CATACTACGGGTCCGCTGTGAAGGGCCGGTTCACCATCTCCAG | | |
| | AGACAATTCCAAGAACACACTGTATCTGCAAATGAACAGCCTG | | |
| | AGAGCCGAGGACACCGCCGTGTATTACTGTGCCAAAAGCAGTT | | |
| | ATGACATTAGTTGGAATGGTCACGTCGAACATCTCGACGCATG | | |
| | GGGCCAGGGGACCCTGGTCACCGTCTCCTCT | | |
| B20 | TCTTCTGAGCTGACTCAGCCTCCTGCTGTGTCTGTGGCCTTGG | VL | SEQ ID NO: 19 |
| | GACAGACAGTCAGGATCACATGCTCCGGGGCTGGAAGTGGCTA | nucleotide | |
| | TGGTTATGGCTGGTACCAGCAGAAGCCAGGACAGGCCCCTGTG | | |
| | ACCGTCATCTATAGCAACGACAAGAGACCCTCCGGGATCCCAG | | |
| | ACCGATTCTCTGGCTCCAGCTCAGGAAACACAGCTTCCTTGAC | | |
| | CATCACTGGGGCTCAGGCCGAAGATGAGGCTGACTATTACTGT | | |
| | GGGACCTACGACAGCACTGATGCTGGTTATGCTATATTTGGGG | | |
| | GCGGGACAAAGCTGACCGTCCTA | | |
| B18 | GAGGTGCAGCTGTTGGAGTCTGGGGGAGGCTTGGTGCAGCCTG | VH | SEQ ID NO: 21 |
| | GGGGGTCCCTGAGACTCTCCTGTGCAGCCTCTGGGTTCGACTT | nucleotide | |
| | CAGCAGTTACGACATGCACTGGGTCCGCCAGGCTCCAGGGAAG | | |
| | GGGCTGGAGTGGGTCTCAGGTATTGATGATTACGGAATTGAAA | | |
| | CATACTACGGGTCCGCTGTGAAGGGCCGGTTCACCATCTCCAG | | |
| | AGACAATTCCAAGAACACACTGTATCTGCAAATGAACAGCCTG | | |
| | AGAGCCGAGGACACCGCCGTGTATTACTGTGCCAAAAGCAGTT | | |
| | ATGACATTAGTTGGAATGGTCACGTCGAACATCTCGACGCATG | | |
| | GGGCCAGGGGACCCTGGTCACCGTCTCCTCT | | |
| B18 | TCTTCTGAGCTGACTCAGCCTCCTGCTGTGTCTGTGGCCTTGG | VL | SEQ ID NO: 23 |
| | GACAGACAGTCAGGATCACATGCCAGGGTGACAGCTCAGGATA | nucleotide | |
| | CGGTTATGGATGGTACCAGCAGAAGCCAGGACAGGCCCCTGTG | | |
| | ACCGTCATCTATGGCAAGAACAATCGTCCGAGCGGGATCCCAG | | |
| | ACCGATTCTCTGGCTCCAGCTCAGGAAACACAGCTTCCTTGAC | | |
| | CATCACTGGGGCTCAGGCCGAAGATGAGGCTGACTATTACTGT | | |
| | GGGACCTACGTCAGCGCATATTATGGTTATGCTATATTTGGGG | | |
| | GCGGGACAAAGCTGACCGTCCTA | | |

TABLE 3
Anti BDNF antibody antigen binding
CDR sequences according to Kabat

| MAb | Sequence | Region | SEQ ID NO. |
|---|---|---|---|
| R3B | SSYDMH | CDRH1 | SEQ ID NO: 7 |
| H1 | | | |
| R3B | GIDDGGSDTYYGSAVKG | CDRH2 | SEQ ID NO: 8 |
| H1 | | | |
| R3B | SSYDISWNGHVENIDA | CDRH3 | SEQ ID NO: 9 |
| H1 | | | |
| R3B | SGAGSGYGYG | CDRL1 | SEQ ID NO: 10 |
| H1 | | | |
| R3B | SNDKRPS | CDRL2 | SEQ ID NO: 11 |
| H1 | | | |
| R3B | GTYDSTDAGYAI | CDRL3 | SEQ ID NO: 12 |
| H1 | | | |
| B30 | SSYDMH | CDRH1 | SEQ ID NO: 25 |
| B30 | GIGDYGIETYYGSAVK | CDRH2 | SEQ ID NO: 26 |
| B30 | SSYDISWNGHVEHIDS | CDRH3 | SEQ ID NO: 27 |
| B30 | SGAGSGYGYG | CDRL1 | SEQ ID NO: 28 |
| B30 | SNDKRPS | CDRL2 | SEQ ID NO: 29 |
| B30 | GTYVSAYYGYAI | CDRL3 | SEQ ID NO: 30 |
| B20 | SSYDMH | CDRH1 | SEQ ID NO: 31 |
| B20 | GIDDYGIETYYGSAVK | CDRH2 | SEQ ID NO: 32 |
| B20 | SSYDISWNGHVEHLDA | CDRH3 | SEQ ID NO: 33 |
| B20 | SGAGSGYGYG | CDRL1 | SEQ ID NO: 34 |
| B20 | SNDKRPS | CDRL2 | SEQ ID NO: 35 |
| B20 | GTYDSTDAGYAI | CDRL3 | SEQ ID NO: 36 |
| B18 | SSYDMH | CDRH1 | SEQ ID NO: 37 |
| B18 | GIDDYGIETYYGSAVK | CDRH2 | SEQ ID NO: 38 |
| B18 | SSYDISWNGHVEHLD | CDRH3 | SEQ ID NO: 39 |
| B18 | QGDSSGYGYG | CDRL1 | SEQ ID NO: 40 |
| B18 | GKNNRPS | CDRL2 | SEQ ID NO: 41 |
| B18 | GTYVSAYYGYAI | CDRL3 | SEQ ID NO: 42 |

The present invention encompasses modifications to the variable regions shown in Table 1 and the CDRs shown in Table 3. For example, the invention includes antibodies comprising functionally equivalent variable regions and CDRs which do not significantly affect their properties as well as variants which have enhanced or decreased activity and/or affinity. For example, the amino acid sequence may be mutated to obtain an antibody with the desired binding affinity to BDNF. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but framework alterations are also contemplated. Conservative substitutions are shown in Table 4 under the heading of “conservative substitutions.” If such substitutions result in a change in biological activity, then more substantial changes, denominated “exemplary substitutions” in Table 4, or as further described below in reference to amino acid classes, may be introduced and the products screened.

To express the anti-BDNF antibodies of the present invention, DNA fragments encoding the antibody, or antigen-binding portion thereof, according to the first aspect can first be obtained using methods known in the art. Various modifications, e.g. mutations, deletions, and/or additions can also be introduced into the DNA sequences using standard methods known to those of skill in the art. For example, mutagenesis can be carried out using standard methods, such as PCR-mediated mutagenesis, in which the mutated nucleotides are incorporated into the PCR primers such that the PCR product contains the desired mutations or site-directed mutagenesis.

TABLE 4
Amino Acid Substitutions

| | Conservative | |
|---|---|---|
| Original Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gin (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu, Val, Met, Ala, Phe, |
| | | Norleucine |
| Leu (L) | Ileu | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Iie; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a beta-sheet or helical conformation, (b) the charge or hydrophobicity of the molecule at the target site, or (c) the bulk of the side chain. Naturally occurring residues are divided into groups based on common side-chain properties:

### (1) Non-polar: Norleucine, Met, Ala, Val, Leu, Ile;

(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and

### (6) Aromatic: Trp, Tyr, Phe, His.

Non-conservative substitutions are made by exchanging a member of one of these classes for another class. One type of substitution, for example, that may be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical. Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

The antibodies may also be modified, e.g. in the variable domains of the heavy and/or light chains, e.g., to alter a binding property of the antibody. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. For example, a mutation may be made in one or more of the CDR regions to increase or decrease the K_{D} of the antibody for BDNF, to increase or decrease k_{off}, or to alter the binding specificity of the antibody.

Techniques in site-directed mutagenesis are well-known in the art. See, e.g., Molecular cloning: a laboratory manual/J. Sambrook, E. F. Fritsch, T. Maniatis Sambrook et al. New York: Cold Spring Harbor Laboratory Press and Current Protocols in Molecular Biology, Ausubel et al., John Wiley & Sons,

In some embodiments the VH comprises the amino acid sequence of antibody R3BH1 SEQ ID NO: 4 or antibody B30 SEQ ID NO:14, or antibody B20 SEQ ID NO: 18 or antibody B18 SEQ ID NO: 22 or a variant thereof with one or several (e.g., 2, 3, 4, 5, 6, 7, 8, 9, 10, 11 or 12) conservative amino acid substitutions in residues that are not within a CDR. In some embodiments the VL comprises the amino acid sequence of antibody R3BH1 SEQ ID NO: 6 or antibody B30 SEQ ID NO:16, or antibody B20 SEQ ID NO: 20 or antibody B18 SEQ ID NO: 24 or a variant thereof with one or several (e.g., 2, 3, 4, 5, 6, 7, 8, 9, 10, 11 or 12) conservative amino acid substitutions in residues that are not within a CDR. In some embodiments the forgoing recited VH and VL of the respective antibody may each comprise one or several (e.g., 2, 3, 4, 5, 6, 7, 8, 9, 10, 11 or 12) conservative amino acid substitutions in residues that are not within a CDR.

A modification or mutation may also be made in a framework region or constant region to increase the half-life of an anti-BDNF antibody. See, e.g., PCT Publication No. WO00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. According to the invention, a single antibody may have mutations in any one or more of the CDRs or framework regions of the variable domain or in the constant region.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jeffe s and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of beta (1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jeffe s et al., Immunological Reviews 163:59-76, 1998. In some embodiments, the constant region is modified as described in Eur. J. Immunol., 1999, 29:2613-2624; PCT Application No. PCT/GB99/01441; and/or UK Patent Application No. 9809951.8.

In some embodiments, an antibody constant region can be modified to avoid interaction with Fc gamma receptor and the complement and immune systems. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

In some embodiments, the constant region is modified as described in Eur. J. Immunol., 1999, 29:2613-2624; PCT Application No. PCT/GB99/01441; and/or UK Patent Application No. 9809951.8. In such embodiments, the Fc can be human IgG₂ or human IgG₄. The Fc can be human IgG2 containing the mutation A330P331 to S330S331 (designated IgG2Δa), in which the amino acid residues are numbered with reference to the wild type IgG2 sequence. Eur. J. Immunol., 1999, 29:2613-2624. In some embodiments, the antibody comprises a constant region of IgG comprising the following mutations (Armour et al., 2003, Molecular Immunology 40 585-593): E233F234L235 to P233V234A235 (IgG₄Δc), in which the numbering is with reference to wild type IgG4. In yet another embodiment, the Fc is human IgG₄ E233F234L235 to P233V234A235 with deletion G236 (IgG₄Δb)—In another embodiment the Fc is any human IgG₄ Fc (IgG₄, IgG Δb or IgG Δ_{c}) containing hinge stabilizing mutation S228 to P228 (Aalberse et al., 2002, Immunology 105, 9-19).

In some embodiments, the antibody comprises a human heavy chain IgG2 constant region comprising the following mutations: A330P331 to S330S331 (amino acid numbering with reference to the wild type IgG2 sequence). Eur. J. Immunol., 1999, 29:2613-2624. In still other embodiments, the constant region is aglycosylated for N-linked glycosylation. In some embodiments, the constant region is aglycosylated for N-linked glycosylation by mutating the oligosaccharide attachment residue and/or flanking residues that are part of the N-glycosylation recognition sequence in the constant region. For example, N-glycosylation site N297 may be mutated to, e.g., A, Q, K, or H. See, Tao et al., J. Immunology 143: 2595-2601, 1989; and Jefferis et al., Immunological Reviews 163:59-76, 1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO 99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding FcRn and/or FcyRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

In some embodiments, the antibody comprises a modified constant region that has increased binding affinity for FcRn and/or an increased serum half-life as compared with the unmodified antibody.

In a process known as “germlining”, certain amino acids in the VH and VL sequences can be mutated to match those found naturally in germline VH and VL sequences. In particular, the amino acid sequences of the framework regions in the VH and VL sequences can be mutated to match the germline sequences to reduce the risk of immunogenicity when the antibody is administered. Germline DNA sequences for human VH and VL genes are known in the art (see e.g., the “Vbase” human germline sequence database; see also Kabat, E. A., et al., 1991, Sequences of Proteins of Immunological Interest, Fifth Edition, U.S. Department of Health and Human Services, NIH Publication No. 91-3242; Tomlinson et al., 1992, J. Mol. Biol. 227:776-798; and Cox et al., 1994, Eur. J. Immunol. 24:827-836).

Another type of amino acid substitution that may be made is to remove potential proteolytic sites in the antibody. Such sites may occur in a CDR or framework region of a variable domain or in the constant region of an antibody. Substitution of cysteine residues and removal of proteolytic sites may decrease the risk of heterogeneity in the antibody product and thus increase its homogeneity. Another type of amino acid substitution is to eliminate asparagine-glycine pairs, which form potential deamidation sites, by altering one or both of the residues. In another example, the C-terminal lysine of the heavy chain of an anti-BDNF antibody of the invention can be cleaved. In various embodiments of the invention, the heavy and light chains of the anti-BDNF antibodies may optionally include a signal sequence.

According to an embodiment of the present invention the isolated monoclonal antibody or an antigen-binding portion thereof binds to BDNF with a binding affinity (K_{D}) of between about 1 pM to about 50,000 pM. According to an embodiment of the present invention the isolated monoclonal antibody or an antigen-binding portion thereof binds to BDNF with a binding constant or K_{D} of between about 1 pM and any of about 10 pM, 20 pM, 30 pM, 40 pM, 50 pM, 60 pM, 70 pM, 80 pM, 90 pM, 100 pM, 110 pM, 120 pM, 130 pM, 140 pM, 150 pM, 160 pM, 170 pM, 180 pM, 190 pM, 200 pM, 250 pM, 300 pM, 350 pM, 400 pM, 450 pM, 500 pM, 550 pM, 600 pM, 650 pM, 700 pM, 750 pM, 800 pM, 850 pM, 900 pM, 950 pM, 1000 pM, 1100 pM, 1200 pM, 1300 pM, 1400 pM, 1500 pM, 1600 pM, 1700 pM, 1800 pM, 1900 pM, 2000 pM, 3000 pM, 4000 pM, 5000 pM, 6000 pM, 7000 pM, 8000 pM, 9000 pM, 10,000 pM, 15,000 pM, 20,000 pM, 25,000 pM, 30,000 pM, 35,000 pM, 40,000 pM, 45,000 pM, 50,000 pM, or 55,000 pM, or less +/−5% or 10% error; for example any one of about 34420 pM, 12106 pM, or 550 pM or 120 pM or 99 pM or less +/−5% or 10% error as measured in an in vitro binding assay for BDNF such as for example SPR (surface plasmon resonance). For example an in vitro binding assay for BDNF may be such as an SPR (surface plasmon resonance) assay, for example wherein the antigen BDNF is immobilised and concentrations of the antibody are introduced and data collected at 37° C. For example the antigen, BDNF, can be directly immobilised on an SPR chip, for example a BIAcore CM5 sensor chip, and serial dilutions of antibody, for example three-fold serial dilutions, may be introduced, for example in a running buffer, (for example, 0.01 M HEPES, 0.15 M NaCl, 3 mM EDTA, and 0.05% v/v surfactant P20 pH 7.4, optionally at a flow rate of 50 μL/min) at 37° C., an association injection, optionally of 47 seconds, is followed by dissociation steps of varying lengths. Data can be collected optionally at data collection rate of 1 Hz and rate constants and binding constants can be determined.

According to an embodiment of the invention, the isolated monoclonal antibody, or an antigen-binding portion thereof, binds selectively to BDNF, and/or it binds selectively to BDNF in comparison to other neurotrophins. In an embodiment, the antibody or an antigen binding portion thereof does not significantly bind to related neurotrophins, such as for example structurally related neurotrophins, for example in comparison to any one or more of Nerve Growth Factor (NGF), Neurotrophin-3 (NT-3) and Neurotrophin-4 (NT-4), or p75NTR for example in comparison to each of Nerve Growth Factor (NGF), Neurotrophin-3 (NT-3), p75NTR, and Neurotrophin-4 (NT-4). Additionally or alternatively the isolated monoclonal antibody, or an antigen-binding portion thereof, binds selectively to BDNF, for example selectively binding to BDNF in comparison to any one or more of the selected chemokines, and does not significantly bind to related chemokines, such as for example chemokines selected from the group CXCL3, CXCL9, CXCL10, CXCL13. According to an embodiment of the invention, the binding affinity (KD) of the isolated monoclonal antibody, or an antigen-binding portion thereof for BDNF is between about 2 and 10,000 tighter than the KD for other neurotrophins and/or chemokines such as for example any one or more of Nerve Growth Factor (NGF), Neurotrophin-3 (NT-3), p75NTR and Neurotrophin-4 (NT-4) and/or to any one or more of the selected chemokines from the group CXCL3, CXCL9, CXCL10, CXCL13 and can be greater than any of about 2, 4, 6, 8, 10, 12, 14, 16, 18, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 800, 825, 850, 875, 900, 925, 950, 975, 1000, 2000, 3000, 4000, 5000, 6000, 7000, 8000. 9000, 10,000 times tighter.

According to an embodiment of the present invention, the isolated monoclonal antibody or an antigen-binding portion thereof, inhibits BDNF binding to the TrKB receptor and/or the p75NTR receptor, for example to both TrKB receptor and p75NTR receptor.

According to an embodiment of the present invention, the isolated monoclonal antibody or an antigen-binding portion thereof can inhibit BDNF binding in-vitro to the TrKB receptor and/or the p75NTR receptor with either an IC50 or a constant (Kᵢ) of between about 0.01 nM to about 300 nM. According to an embodiment of the invention, the isolated monoclonal antibody or an antigen-binding portion thereof can inhibit BDNF binding to the TrKB receptor and/or the p75NTR receptor with IC50 or the inhibition constant (Ki) of about or less than about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 nM, +/−5% or 10% error as measured in a suitable activity assay such as for example an SPR (surface plasmon resonance) or HTRF (Homogeneous Time Resolved Fluorescence) assay as described herein. In an embodiment, the IC50 or Ki is less than about 0.5 nM and may be between about 0.1 and about 0.5 nM+/−5% or 10% error. The inhibition of BDNF binding in-vitro to the TrKB receptor and/or the p75NTR receptor can be measured by an in-vitro binding assay for BDNF such as for example SPR (surface plasmon resonance) or HTRF (Homogeneous Time Resolved Fluorescence) assay as described herein. A homogenous time-resolved fluorescence assay (HTRF assay) can be used to identify anti-BDNF antibodies that are capable of displacing BDNF bound TrkB receptor. For example a recombinant TrkB-Fc labelled with europium cryptate is added to an assay mixture containing biotinylated human BDNF and a dilution series of anti-BDNF antibody is added and a fluorescence reading measured from which the IC50 may be calculated. The assay may be conducted at room temperature, for example in an assay buffer at pH7.5 at room temperature, for example an assay buffer of 50 mM sodium phosphate, pH 7.5, 400 mM potassium fluoride, and 0.1% BSA (w/v). Reactions can proceed for a period, for example 3 hours before taking data readings. Data can be obtained with excitation at 340 nm and two emission readings at 615 nm and 665 nm and readings can be expressed as a ratio of fluorescence at 665/615, optionally using an EnVision MultiLabel Plate Reader. Alternatively the ability of an anti-BDNF antibody to inhibit binding of BDNF to p75NTR receptor can be determined using an SPR assay at room temperature for example run on the BIAcore T200. For example the p75NTR can be immobilized onto the flow cell, increasing concentrations of anti-BDNF antibody are added in the presence of BDNF and signal detected from which IC50 for inhibition of BDNF-p75NTR interaction can be determined.

According to an embodiment of the present invention, the isolated monoclonal antibody or an antigen-binding portion thereof can inhibit BDNF activity, or activity at or activation at the TrKB receptor and/or the p75NTR receptor, for example can inhibit the ability to bind a BDNF receptor (such as p75NTR and/or trkB) and/or the ability to promote trkB and/or p75NTR receptor dimerization and/or autophosphorylation and/or the ability to activate an BDNF receptor signalling pathway; and aforementioned ability to promote or effect cell or neuron biology and/or mediate pain. According to an embodiment of the present invention, the isolated monoclonal antibody or an antigen-binding portion thereof can inhibit BDNF activity and/or binding to the TrKB receptor and/or the p75NTR receptor and/or activation of BDNF receptor signalling pathways, with either an IC50 or a constant (Kᵢ) of between about 0.01 nM to about 300 nM. According to an embodiment of the invention, the isolated monoclonal antibody or an antigen-binding portion thereof can inhibit BDNF binding to the TrKB receptor and/or the p75NTR receptor with IC50 or the inhibition constant (Ki) of about or less than about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 nM, +/−5% or 10% error as measured in a suitable activity assay such as the pERK or pTrkB assay described herein. In an embodiment, the isolated monoclonal antibody or an antigen-binding portion thereof can inhibit BDNF activity at and/or binding to and/or activation of the TrKB receptor and/or the p75NTR receptor with either an IC50 or a constant (Kᵢ) of any one of about 262, 53.6, 24, 11.7, 7.6, 4.7, 4.4, 1.3, 1.1, 0.95, 0.54, 0.31 and 0.29 nM+/−5% or 10% error as measured in a suitable activity assay such as the pERK or Enzyme Fragment Complementation (EFC) assay described herein. For example anti-BDNF antibody inhibition of BDNF activity or activation at the TrkB and p75NTR receptors can be measured in TrkB/p75NTR expressing cells using a pERK (phospho-extracellular signal-regulated kinase) assay. IC50 is measured from determination of reduced phosphorylation of ERK in the presence of anti-BDNF antibody added to BDNF and cells expressing TrkB+p75NTR at room temperature. Serial dilutions of the anti-BDNF antibody can be added to cells expressing TrkB+p75NTR, for example U2OS cells (DiscoverX Corp.) in the presence of BDNF at room temperature followed by addition of reagents containing extracellular signal-regulated kinase, ERK. IC50 can be determined from levels of binding of BDNF to the TrkB receptor are determined from receptor dimerization and transphosphorylation of tyrosine residues of Erk can be detected using a labelled anti-phospho-ERK antibody and a labelled anti-ERK antibody.

Alternatively anti-BDNF antibody inhibition of BDNF activity or activation at the TrkB and p75NTR receptors can be measured in an Enzyme Fragment Complementation (EFC) assay, for example using the PathHunter assay (DiscoverX). IC50 can be determined from chemiluminescent measurement of levels of a specific protein-protein interaction in TrkB/p75NTR expressing cells (the protein-protein interaction can be between a small peptide epitope (ProLink) expressed on the C-terminus of TrkB and co-expressed enzyme acceptor (EA) attached to a SH2 phospho-tyrosine binding domain), for example U2OS cells, in the presence of BDNF and anti-BDNF antibody, for example serially diluted antibody samples, at room temperature. Optionally the chemiluminescence is read using an Envision plate reader (Perkin Elmer).

According to an embodiment of the invention, the isolated monoclonal antibody, or an antigen-binding portion thereof, specifically binds to BDNF in-vitro and/or specifically binds to BDNF in-vivo. The isolated monoclonal antibody or an antigen-binding portion thereof, can bind in a dose or concentration dependant manner to BDNF and/or can form a stable complex. According to an embodiment of the invention, the isolated monoclonal antibody, or an antigen-binding portion thereof, can form a complex with BDNF which can have a half life in-vitro and/or in-vivo and/or in biological fluid of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 hours+/−1 hour. The isolated monoclonal antibody or an antigen-binding portion thereof, can bind in a dose or concentration dependant manner to BDNF and/or can form a stable complex. According to an embodiment of the invention, the isolated monoclonal antibody, or an antigen-binding portion thereof, can form a complex with BDNF which can have a half life in-vitro and/or in-vivo and/or in biological fluid of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 days+/−1 day. In an embodiment, the half life is about or more than any one of about 5 days, 6 days, 20 days, 26 days, 27 days.

According to the foregoing embodiments of the invention the isolated monoclonal antibody, or an antigen-binding portion thereof, BDNF complex has a half life in-vivo or in biological fluid of about or more than 6 days. The stability in-vitro can be measured at about physiological pH, in a buffered aqueous solution, for example at 20° C. or 37° C., for example by SPR (surface Plasmon resonance, BIACORE), ELISA or radioimmunoassay to quantify the levels of active antibody by target BDNF binding or alternatively by determination of the soluble antibody level in solution using spectrophotometry. According to the foregoing embodiments, the in-vivo half life can be half life in a rat, mouse or human body or biological fluid thereof, for example human. The half life can also determined from serum or plasma measurements of the antibody BDNF complex levels following introduction of the antibody into a biological fluid sample or its administration in-vivo for example by intravenous or subcutaneous injection.

A prolonged half life of the antibody BDNF complex and higher stability in-vivo for example in serum is desirable as it permits a dosage regime of less frequent dosing and/or lower dosing levels hence reducing risk of any potential toxicity or side effects in-vivo. High stability of the antibody BDNF complex is an indicator of higher potency and has the mentioned benefit that the antibody can be used at lower dosage amounts than a less specific and/or less selective and/or less potent antibody to achieve the same therapeutic efficacy hence reducing potential toxicity or side effects in-vivo.

The antibody, or antigen-binding portion thereof can have a half life in-vivo of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 40, 42, 44, 426, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, or 600 hours+/−1 hour. For example the antibody, or antigen-binding portion thereof can have a half life in-vivo of between about 163 and 540 hours and or about or more than about 163 hours. The antibody, or antigen-binding portion thereof can have a half life in-vivo of about or more than any one of about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 days+/−1 day, for example the antibody, or antigen-binding portion thereof has a half life in-vivo of between about 6 and 22 days, for example of about or more than about 6 days.

According to the foregoing embodiments, the in-vivo half life can be the half life in rat, mouse or human body or biological fluid thereof. The half life can be determined from plasma or serum measurements of the levels of the antibody, or antigen-binding portion thereof following administration in-vivo for example by intravenous or subcutaneous injection.

According to an embodiment of the present invention, the antibody or an antigen-binding portion thereof, can be human, humanised or chimeric.

The antibody or an antigen-binding portion thereof can have an isotype subclass selected from the group consisting of IgG1, of IgG₂, IgG₄, IgG_{2Δa}, IgG_{4Δb}, IgG_{4Δc}, IgG₄ S228P, IgG_{4Δb} S228P and IgG_{4Δc} S228P. The antibody or an antigen-binding portion thereof, can be a full length-antibody of an IgG1, of IgG₂, IgG₄, IgG_{2Δa}, IgG_{4Δb}, IgG_{4Δc}, IgG₄ S228P, IgG_{4Δb} S228P or IgG_{4Δc} S228P isotype. The antibody or an antigen-binding portion thereof, may be a single chain antibody, a Fab fragment, a F(ab)₂ fragment, a Fv fragment, a tetrameric antibody, a tetravalent antibody, a multispecific antibody, a domain-specific antibody, a single domain antibody, or a fusion protein. The invention also provides a bispecific molecule comprising the antibody, or antigen-binding portion thereof, of the invention, linked to a second functional moiety having a different binding specificity than said antibody, or antigen binding portion thereof.

### Immunoconjugates

According to a second aspect of the present invention there is provided an immunoconjugate comprising the antibody, or antigen-binding portion thereof according to the first aspect linked to a therapeutic agent.

Representative therapeutic agents include cytotoxins, radioisotopes, chemotherapeutic agents, immunomodulatory agents, anti-angiogenic agents, antiproliferative agents, pro-apoptotic agents, and cytostatic and cytolytic enzymes (e.g., RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. For example, selected radioisotopes are also cytotoxins. Therapeutic agents may be prepared as pharmaceutically acceptable salts, acids or derivatives of any of the above. Generally, conjugates having a radioisotope as the drug are referred to as radioimmunoconjugates and those having a chemotherapeutic agent as the drug are referred to as chemoimmunoconjugates.

Examples of suitable therapeutic agents for use in immunoconjugates include the taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers (i.e., for photodynamic therapy) can also be used in immunoconjugates. In addition, conjugates can be made using secondary carriers as the cytotoxic agent, such as liposomes or polymers,

Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins. Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

In particular embodiments of the invention, a cytotoxin is an antibiotic such as a calicheamicin, also called the LL-E33288 complex, for example, gamma-calicheamicin (gamma 1) or N-acetyl gamma-calicheamicin. See U.S. Pat. No. 4,970,198. Additional examples of calicheamicins suitable for use in preparing antibody/drug conjugates of the invention are disclosed in U.S. Pat. Nos. 4,671,958; 5,053,394; 5,037,651; 5,079,233; and 5,108,912. These compounds contain a methyltrisulfide that may be reacted with appropriate thiols to form disulfides, at the same time introducing a functional group such as a hydrazide or other functional group that is useful for conjugating calicheamicin to an anti-5T4 antibody. Disulfide analogs of calicheamicin can also be used, for example, analogs described in U.S. Pat. Nos. 5,606,040 and 5,770,710.

The antibody of the invention may comprise a high energy radioisotope. The isotope may be directly bound to the antibody, for example, at a cysteine residue present in the antibody, or a chelator may be used to mediate the binding of the antibody and the radioisotope. Radioisotopes suitable for radiotherapy include but are not limited to alpha-emitters, beta-emitters, and auger electrons. For diagnostic applications, useful radioisotopes include positron emitters and gamma-emitters. The antibody of the invention may further be iodinated, for example, on a tyrosine residue of the antibody, to facilitate detection or therapeutic effect of the antibody. Representative radioisotopes that may be conjugated to an anti-5T4 antibody include ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, “technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³indium, ^{99m}rhenium, ¹⁰⁵rhenium, ¹⁰¹rhenium, ¹⁸⁶rhenium, ¹⁸⁸rhenium, ¹²¹mtelluhum, “technetium, ^{122m}tellurium, ^{125m}telluhum, ¹⁶⁵thulium, ¹⁶⁷thulium, ¹⁶⁸thulium, “yttrium, and nitride or oxide forms derived there from. Other suitable radioisotopes include alpha emitters, such as ²¹³bismuth, ²¹³lead, and ²²⁵actinium.

Antibody/drug conjugates of the invention may include immunomodulators, i.e., agents that elicit an immune response, including humoral immune responses (e.g. production of antigen-specific antibodies) and cell-mediated immune responses (e.g. lymphocyte proliferation). Representative immunomodulatory agents include cytokines, xanthines, interleukins, interferons, and growth factors (e.g., TNF, CSF, GM-CSF and G-CSF), and hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Suitable immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, thoxifene, keoxifene, LY 1 17018, onapnstone, and toremifene; and anti-androgens such as flutamide, nilutamide, bicalutamide, leuprolide, and goserelin; and anti-adrenal agents. Representative immunosuppressive agents include 2-amino-6-aryl-5-substituted pyhmidines, azathiophne, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-IL10 antibodies, anti-TNFa antibodies, anti-IL2 antibodies), streptokinase, TGF beta, rapamycin, T-cell receptor, T-cell receptor fragments, and T cell receptor antibodies. Additional drugs useful in the invention include anti-angiogenic agents that inhibit blood vessel formation, for example, fa rnesyl transferase inhibitors, COX-2 inhibitors, VEGF inhibitors, bFGF inhibitors, steroid sulphatase inhibitors (e.g., 2-methoxyoestradiol bis-sulphamate (2-MeOE2bisMATE)), interleukin-24, thrombospondin, metallospondin proteins, class I interferons, interleukin 12, protamine, angiostatin, laminin, endostatin, and prolactin fragments.

Suitable anti-proliferative agents and pro-apoptotic agents include activators of PPAR-gamma (e.g., cyclopentenone prostaglandins (cyPGs)), retinoids, triterpinoids (e.g., cycloartane, lupane, ursane, oleanane, fhedelane, dammarane, cucurbitacin, and limonoid thterpenoids), inhibitors of EGF receptor (e.g., HER4), rampamycin, CALCITRIOL® (1,25-dihydroxycholecalciferol (vitamin D)), aromatase inhibitors (FEMARA® (letrozone)), telomerase inhibitors, iron chelators (e.g., 3-aminopyridine-2-carboxaldehyde thiosemicarbazone (Thapine)), apoptin (viral protein 3—VP3 from chicken aneamia virus), inhibitors of Bcl-2 and Bcl-X(L), TNF-alpha, FAS ligand, TNF-related apoptosis-inducing ligand (TRAI L/Apo2L), activators of TNF-alpha/FAS ligand/TNF-related apoptosis-inducing ligand (TRAIL/Apo2L) signaling, and inhibitors of PI3K-Akt survival pathway signaling (e.g., UCN-01 and geldanamycin).

Representative chemotherapeutic agents include alkylating agents such as thiotepa and cyclosphosphamide; alkyl sulfonates such as busulfan, improsulfan and piposulfan; aziidines such as benzodopa, carboquone, meturedopa, and uredopa; ethylenimines and methylamelamines including altretamine, triethylenemelamine, thethylenephosphoramide, thethylenethiophosphoramide and thmethylolomelamine; nitrogen mustards such as chlorambucil, chlornaphazine, cholophosphamide, estramustine, ifosfamide, mechiorethamine, mechiorethamine oxide hydrochloride, melphalan, novembichin, phenestehne, prednimustine, trofosfarnide, uracil mustard; nitrosureas such as carmustine, chlorozotocin, fotemustine, lomustine, nimustine, ranimustine; antibiotics such as aclacinomysins, actinomycin, authramycin, azasehne, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonighn, streptozocin, tubercidin, ubenimex, zinostatin, zorubicin; anti-metabolites such as methotrexate and 5-fluorouracil (5-FU); folic acid analogues such as denoptehn, methotrexate, pteropterin, trimetrexate; purine analogs such as fludarabine, 6-mercaptopurine, thiamiprine, thioguanine; pyhmidine analogs such as ancitabine, azacitidine, 6-azauhdine, carmofur, cytarabine, dideoxyuridine, doxifluhdine, enocitabine, floxuridine, 5-EU; androgens such as calusterone, dromostanolone propionate, epitiostanol, mepitiostane, testolactone; anti-adrenal such as arninoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; thaziquone; 2, 2′, 2′-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g. paclitaxel (TAXOL®, Bristol-Myers Squibb Oncology of Princeton, N.J.) and doxetaxel (TAXOTERE®, Rhone-Poulenc Rorer of Antony, France); chiorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aininopterin; xeloda; ibandronate; CPT-1 1; topoisomerase inhibitor RFS 2000; difluoromethylornithine (DMFO); retinoic acid; esperamicins; and capecitabine.

Additional therapeutic agents that may be conjugated to the antibody of the invention include photosensitizing agents (U.S. Patent Publication No. 2002/0197262 and U.S. Pat. No. 5,952,329) for photodynamic therapy; magnetic particles for thermotherapy (U.S. Patent Publication No. 2003/0032995); binding agents, such as peptides, ligands, cell adhesion ligands, etc., and prodrugs such as phosphate-containing prodrugs, thiophosphate-containing prodrugs, sulfate containing prodrugs, peptide containing prodrugs, beta-lactam-containing prodrugs, substituted phenoxyacetamide-containing prodrugs or substituted phenylacetamide-containing prodrugs, 5-fluorocytosine and other 5-fluorouhdine prodrugs that may be converted to the more active cytotoxic free drug. The antibody of the may comprise a detectable label used to detect the presence of BDNF-expressing cells in vitro or in vivo.

The antibody of the invention may be linked to radioisotopes that are detectable in vivo, such as those labels that are detectable using scintigraphy, magnetic resonance imaging, or ultrasound. Useful scintigraphic labels include positron emitters and gamma-emitters. Representative contrast agents for magnetic source imaging are paramagnetic or superparamagnetic ions such as, iron, copper, manganese, chromium, erbium, europium, dysprosium, holmium and gadolinium, iron oxide particles, and water soluble contrast agents. For in vitro use, useful detectable labels include fluorophores, detectable epitopes or binding agents, and radioactive labels.

### Nucleic Acid Molecules, Vectors, Host Cells

According to a third aspect of the invention there is provided a nucleic acid molecule encoding the antibody, or antigen-binding portion thereof, according to the first aspect. The nucleic acid molecule can be for use as a medicament and/or for use in the prevention and/or treatment of pain, including chronic or acute pain.

According to an embodiment of the present invention the nucleic acid molecule may further comprise a region encoding a signal sequence, for example an immunoglobulin signal sequence for example a DNA or RNA sequence.

According to a fourth aspect of the invention there is provided a replicable expression vector for transfecting a cell, the vector comprising the nucleic acid molecule of the third aspect. In an embodiment, the vector is a viral vector. The vector can be for use as a medicament and/or for use in the prevention and/or treatment of pain.

Further according to the third or fourth aspects of the invention there is provided a method of expressing the nucleic acid molecule or the vector of the invention to produce or secrete the antibody, or antigen-binding portion thereof. The method can comprise the introduction of the nucleic acid molecule or vector into a cell and expression of the nucleic acid therein to produce or secrete the antibody, or antigen-binding portion thereof. The nucleic acid molecule or vector can be introduced into the cell in-vitro alternatively in-vivo. The expressed antibody, or antigen-binding portion thereof, can be expressed in-vitro, optionally further isolated and purified, alternatively the expressed antibody, or antigen-binding portion thereof, can be expressed in-vivo, the in-vivo expression can constitute gene therapy. The vector can be a replicable expression vector, optionally for transfecting a mammalian cell, for example the vector can be a viral vector.

According to a fifth aspect of the invention there is provided a host cell harbouring the nucleic acid molecule or vector of either the third or fourth aspect, for example the cell can be a eukaryotic cell or a prokaryotic cell, for example a bacterial cell a yeast cell or a mammalian cell. In an embodiment, the host cell is a mammalian cell.

Methods of Therapy with the Anti-BDNF Antibodies and Immunoconjugates, and Pharmaceutical Compositions of the Invention

According to a sixth aspect of the invention there is provided the antibody, or antigen-binding portion thereof, according to the first aspect or the immunoconjugate according to the second aspect, or the nucleic acid or vector according to the third and fourth aspects or the combination of the seventh aspect or the pharmaceutical composition according to the ninth aspect, for use in the treatment of pain, or for the prevention and/or treatment of pain and/or symptoms of pain or for ameliorating, controlling, reducing incidence of, or delaying the development or progression of pain and/or symptoms of pain. In an embodiment the pain or symptom of pain is selected from:

(a) acute pain and/or spontaneous pain,
(b) chronic pain and or on-going pain,
(c) inflammatory pain including any one of arthritic pain, pain resulting from osteoarthritis or rheumatoid arthritis, resulting from inflammatory bowel diseases, psoriasis and eczema
(d) nociceptive pain,
(e) neuropathic pain, including painful diabetic neuropathy traumatic nerve injury, or pain associated with post-herpetic neuralgia, trigeminal neuralgia, HIV neuropathy, chemotherapy induced neuropathy,
(f) hyperalgesia,
(g) allodynia,
(h) central pain, central post-stroke pain, pain resulting from multiple sclerosis, pain resulting from spinal cord injury, or pain resulting from Parkinson's disease or epilepsy,
(i) cancer pain,
(j) post-operative pain,
(k) visceral pain, including digestive visceral pain and non-digestive visceral pain, pain due to gastrointestinal (GI) disorders, pain resulting from functional bowel disorders (FBD), pain resulting from inflammatory bowel diseases (IBD), pain resulting from bladder conditions including interstitial cystitis, painful bladder syndrome, overactive bladder, pain resulting from dysmenorrhea, endometriosis, pelvic pain, or pancreatitis,
(l) musculo-skeletal pain, myalgia, fibromyalgia, spondylitis, sero-negative (non-rheumatoid) arthropathies, non-articular rheumatism, dystrophinopathy, Glycogenolysis, polymyositis, pyomyositis,
(m) heart or vascular pain, pain due to angina, myocardical infarction, mitral stenosis, pericarditis, Raynaud's phenomenon, scleredoma, scleredoma or skeletal muscle ischemia,
(n) head pain including migraine, migraine with aura, migraine without aura cluster headache, tension-type headache.
(o) orofacial pain, including dental pain, temporomandibular myofascial pain or tinnitus, or
(p) back pain, bursitis, menstrual pain, migraine, referred pain, trigeminal neuralgia, hypersensitisation, pain resulting from spinal trauma and/or intravertebral disc degeneration or stroke.

According to a seventh aspect of the invention there is provided the antibody, or antigen-binding portion thereof, according to the first aspect or the immunoconjugate according to the second aspect, or the nucleic acid or vector according to the third and fourth aspects for use according to the sixth aspect or pharmaceutical composition according to the ninth aspect, wherein said antibody, or antigen-binding portion thereof, immunoconjugate, nucleic acid or vector is for separate, sequential or simultaneous use in a combination with a second pharmacologically active compound. For example the second pharmacologically active compound of the combination is selected from;
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline; a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine; Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine; or
a 5-HT3 antagonist, such as ondansetron;
   and the pharmaceutically acceptable salts and solvates thereof.

According to an eighth aspect of the present invention there is provided a method of treating, preventing, ameliorating, controlling, reducing incidence of, or delaying the development or progression of pain or any of the foregoing pain and/or symptoms of pain in an individual, comprising administration to the individual of an effective amount of the antibody, or antigen-binding portion thereof, according to the first aspect or the immunoconjugate according to the second aspect, or the nucleic acid or vector according to the third and fourth aspects or the combination of the seventh aspect or a pharmaceutical composition according to the ninth aspect. For example the individual is a human, or a companion animal such as a horse, cat or dog or a farm animal such as a sheep, cow or pig.

According to a ninth aspect of the present invention there is provided a pharmaceutical composition optionally for any one or more of treating, preventing, ameliorating, controlling, reducing incidence of, or delaying the development or progression of pain or any of the foregoing pain/or symptoms, comprising the antibody, or antigen-binding portion thereof, according to the first aspect or the immunoconjugate according to the second aspect, or the nucleic acid or vector according to the third and fourth aspects or the combination of the seventh aspect and a pharmaceutically acceptable carrier and/or an excipient.

The antibody, or antigen-binding portion thereof, according to the first, second or seventh aspects or the embodiments thereof, or the nucleic acid molecule or vector according to the third and fourth aspects or the combination of the seventh aspect or the pharmaceutical composition of the ninth aspect is prepared for or suitable for oral, sublingual, buccal, topical, rectal, inhalation, transdermal, subcutaneous, intravenous, intra-arterial, intramuscular, intracardiac, intraosseous, intradermal, intraperitoneal, transmucosal, vaginal, intravitreal, intra-articular, peri-articular, local or epicutaneous administration.

The antibody, or antigen-binding portion thereof, according to the first aspect or the immunoconjugate according to the second aspect, or the nucleic acid or vector according to the third and fourth aspects or the combination of the seventh aspect or the pharmaceutical composition of the ninth aspect is prepared for or suitable for administration prior to and/or during and/or after the onset of pain or other aforementioned conditions for therapy or for such use.

The antibody, or antigen-binding portion thereof, according to the first aspect or the immunoconjugate according to the second aspect, or the nucleic acid or vector according to the third and fourth aspects or the combination of the seventh aspect or the pharmaceutical composition of the ninth aspect is for, or is prepared for, administration between once to 7 times per week, for example around once twice, three, four, five six or seven times per week, by further example between once to four times per month, or between once to six times per 6 month period, or once to twelve times per year. The medicament is, or is prepared to be, peripherally administered in a period selected from: once daily, once every two, three, four, five or six days, weekly, once every two weeks, once every three weeks, monthly, once every two months, once every three months, once every four months, once every five months, once every six months, once every seven months, once every eight months, once every nine months, once every ten months, once every eleven months or yearly.

Furthermore the antibody, or antigen-binding portion thereof, according to the first aspect or the immunoconjugate according to the second aspect, or the nucleic acid or vector according to the third and fourth aspects or the combination of the seventh aspect or the pharmaceutical composition of the ninth aspect can be, is, or is prepared to be, peripherally administered via a route selected from one or more of; orally, sublingually, buccally, topically, rectally, via inhalation, transdermally, subcutaneously, intravenously, intra-arterially or intramuscularly, via intracardiac administration, intraosseously, intradermally, intraperitoneally, transmucosally, vaginally, intravitreally, epicutaneously, intra-articularly, intravesically, intrathecally, peri-articularly or locally. In an embodiment the administration is intravenous or subcutaneous administration.

The antibody, or antigen-binding portion thereof, according to the first aspect or the immunoconjugate according to the second aspect, or the nucleic acid or vector according to the third and fourth aspects or the combination of the seventh aspect or the pharmaceutical composition of the ninth aspect is for, or is prepared for, administration at a concentration of between about 0.1 to about 200 mg/ml; for example at any one of about 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190 or 200 mg/ml+/−about 10% error, for example at about 50 mg/ml.

The antibody, or antigen-binding portion thereof, according to the first aspect or the immunoconjugate according to the second aspect, or the nucleic acid or vector according to the third and fourth aspects or the combination of the seventh aspect or the pharmaceutical composition of the ninth aspect is for, or is prepared for, administration at a concentration of between about 0.01 to about 200 mg/kg of body weight; for example at any one of about 0.1, 0.5, 1, 5, 10, 15 20, 25, 30, 35, 40, 45, 50, 55, 60, 65, 70, 75, 80, 85, 90, 95, 100, 110, 120, 130, 140, 150, 160, 170, 180, 190 or about 200 mg/kg of body weight+/−about 10% error, for example at about 10 mg/kg.

The anti-BDNF antibody of the invention can be administered to an individual via any suitable route. It should be apparent to a person skilled in the art that the examples described herein are not intended to be limiting but to be illustrative of the techniques available. Accordingly, in some embodiments, the anti-BDNF antibody of the invention is administered to an individual in accordance with known methods, such as intravenous administration, e.g., as a bolus or by continuous infusion over a period of time, by intramuscular, intraperitoneal, intracerebrospinal, transdermal, subcutaneous, intraarticular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution.

Alternatively, anti-BDNF antibody of the invention can be aerosolized using a fluorocarbon formulation and a metered dose inhaler, or inhaled as a lyophilized and milled powder.

In some embodiments, an anti-BDNF antibody of the invention is administered via site-specific or targeted local delivery techniques. Examples of site-specific or targeted local delivery techniques include various implantable depot sources of the anti-BDNF antibody of the invention or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of an anti-BDNF antibody of the invention may be used for administration. In some embodiments, the anti-BDNF antibody of the invention may be administered neat. In some embodiments, anti-BDNF antibody of the invention and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

An anti-BDNF antibody of the invention can be administered using any suitable method, including by injection (e.g., intraperitoneally, intravenously, subcutaneously, intramuscularly, etc.). Anti-BDNF antibodies can also be administered topically or via inhalation, as described herein. Generally, for administration of anti-BDNF antibodies, an initial candidate dosage can be about 2 mg/kg. For the purpose of the present invention, a typical daily dosage might range from about any of 3 μg/kg to 30 μg/kg to 300 μg/kg to 3 mg/kg, to 30 mg/kg, to 100 mg/kg or more, depending on the factors mentioned above. For example, dosage of about 1 mg/kg, about 2.5 mg/kg, about 5 mg/kg, about 10 mg/kg, and about 25 mg/kg may be used. For repeated administrations over several days or longer, depending on the condition, the treatment is sustained until a desired suppression of symptoms occurs or until sufficient therapeutic levels are achieved, for example, to reduce, prevent or treat pain. The progress of this therapy is easily monitored by conventional techniques and assays. The dosing regimen (including the anti-BDNF antibody of the invention used) can vary over time.

For the purpose of the present invention, the appropriate dosage of an anti-BDNF antibody will depend on the antibody (or compositions thereof) employed, the type and severity of pain to be treated, whether the agent is administered for preventive or therapeutic purposes, previous therapy, the patient's clinical history and response to the agent, the amount of pain present the patient's clearance rate for the administered agent, and the discretion of the attending physician. Typically the clinician will administer an anti-BDNF antibody until a dosage is reached that achieves the desired result in treating and/or preventing pain. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on prevention and/or treatment and/or suppression and/or amelioration and/or delay of pain. Alternatively, sustained continuous release formulations of anti-BDNF antagonist antibodies may be appropriate. Various formulations and devices for achieving sustained release are known in the art.

In one embodiment, dosages for an antagonist antibody may be determined empirically in individuals who have been given one or more administration(s) of an antagonist antibody. Individuals are given incremental dosages of an anti-BDNF antagonist antibody. To assess efficacy, an indicator of the disease can be followed.

Administration of an anti-BDNF antibody of the invention in accordance with the present invention can be continuous or intermittent, depending, for example, upon the recipient's physiological condition, whether the purpose of the administration is therapeutic or prophylactic, and other factors known to skilled practitioners. The administration of an anti-BDNF antibody of the invention may be essentially continuous over a preselected period of time or may be in a series of spaced doses.

In some embodiments, more than one anti-BDNF antibody of the invention may be present. At least one, at least two, at least three, at least four, at least five different, or more antagonist antibodies can be present. Generally, those anti-BDNF antagonist antibodies may have complementary activities that do not adversely affect each other. An anti-BDNF antibody of the invention can also be used in conjunction with other antibodies to BDNF, and/or other pain therapies. An anti-BDNF antibody of the invention can also be used in conjunction with other agents that serve to enhance and/or complement the effectiveness of the agents.

In some embodiments, the anti-BDNF antibody of the invention may be administered or provided for administration separately, sequentially or simultaneously in combination with a further pharmacologically active compound as described according to the seventh aspect of the present invention including the pharmacologically active compounds referred to therein. In some embodiments, an anti-BDNF antibody of the invention is used in conjunction with a further pharmacologically active compound. Alternatively, the therapeutic administration of the anti-BDNF antibody of the invention may precede or follow the further pharmacologically active compound treatment by intervals ranging from minutes to weeks. In embodiments where the anti-BDNF antibody of the invention and the further pharmacologically active compound are administered separately, one would generally ensure that a significant period of time did not expire between each delivery, such that the anti-BDNF antibody of the invention and the pharmacologically active compound would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Therapeutic formulations of the anti-BDNF antibody of the invention used in accordance with the present invention are prepared for storage by mixing an antibody having the desired degree of purity with optional pharmaceutically acceptable carriers, excipients or stabilizers (Remington, The Science and Practice of Pharmacy 20th Ed), Mack Publishing, 2000), in the form of lyophilized formulations or aqueous solutions.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the anti-BDNF antibody of the invention are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylnnethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations for use in in-vivo administration must be sterile. This is readily accomplished by, for example, filtration through sterile filtration membranes. Therapeutic anti-BDNF antibody of the invention compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5 percent surface-active agent, and can be between 0.1 and 2.5 percent. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20 percent oil, for example, between 5 and 20 percent. The fat emulsion can comprise fat droplets between 0.1 and 1.0 micrometers particularly 0.1 and 0.5 micrometers and have a pH in the range of 5.5 to 8.0.

The emulsion compositions can be those prepared by mixing an anti-BDNF antibody of the invention with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Kits

According to a tenth aspect of the present invention there is provided a kit comprising:

(a) the antibody, or antigen-binding portion thereof, according to the first aspect or the immunoconjugate according to the second aspect, or the nucleic acid or vector according to the third and fourth aspects or the combination of the seventh aspect or the pharmaceutical composition of the ninth aspect; and
(b) instructions for the administration of an effective amount of said antibody or antigen binding portion thereof, immunoconjugate, nucleic acid molecule, vector or pharmaceutical composition to an individual for any one or more of the prevention or treatment of pain and/or symptoms of pain or for ameliorating, controlling, reducing incidence of, or delaying the development or progression of pain and/or symptoms of pain.

The kit may include one or more containers containing the antibody or antigen binding portion thereof, immunoconjugate, nucleic acid molecule, vector or pharmaceutical composition described herein and instructions for use in accordance with any of the methods and uses of the invention. The kit may further comprise a description of selecting an individual suitable for treatment based on identifying whether that individual has a pain or a symptom of pain or is at risk of having such. The instructions for the administration of the pharmaceutical composition may include information as to dosage, dosing schedule and routes of administration for the intended treatment.

Generally, kit instructions comprise a description of administration of the anti-BDNF antibody for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In some embodiments, the antibody is a human antibody. In some embodiments, the antibody is a humanized antibody. In some embodiments, the antibody is a monoclonal antibody. The instructions relating to the use of an anti-BDNF antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar™ or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-BDNF antibody. The container may further comprise a second pharmaceutically active agent.

Kits may optionally provide additional components such as buffers and interpretive information. Normally, the kit comprises a container and a label or package insert(s) on or associated with the container.

The invention also provides diagonistic kits comprising any or all of the antibodies described herein. The diagonistic kits are useful for, for example, detecting the presence of BDNF in a sample. In some embodiments, a diagnostic kit can be used to identify an individual at risk of developing pain. In some embodiments, a diagnostic kit can be use to detect the presence of BDNF in an individual.

Diagnostic kits of the invention include one or more containers comprising an anti-BDNF antibody described herein and instructions for use in accordance with any of the methods of the invention described herein. Generally, these instructions comprise a description of use of the anti-BDNF antagonist to detect the presence of BDNF in individuals at risk for, or suspected of having, pain. In some embodiments, an exemplary diagonistic kit can be configured to contain reagents such as, for example, an anti-BDNF antibody, a negative control sample, a positive control sample, and directions for using the kit.

### Medical Use

According to an eleventh aspect of the present invention there is provided the antibody, or antigen-binding portion thereof, according to the first or the immunoconjugate according to the second aspect, or the nucleic acid or vector according to the third and fourth aspects or the combination of the seventh aspect or the pharmaceutical composition of the ninth aspect for use in any one or more of the prevention or treatment or for ameliorating, controlling, reducing incidence of, or delaying the development or progression of a condition or the symptoms of a condition associated with BDNF.

According to a twelfth aspect of the present invention there is provided the use of the antibody, or antigen-binding portion thereof, according to the first or the immunoconjugate according to the second aspect, or the nucleic acid or vector according to the third and fourth aspects or the combination of the seventh aspect or the pharmaceutical composition of the ninth aspect for the manufacture of a medicament for the prevention or treatment or for ameliorating, controlling, reducing incidence of, or delaying the development or progression of a condition or the symptoms of a condition associated with BDNF.

### Biological Deposit

Representative materials of the present invention were deposited in the American Type Culture Collection, 10801 University Boulevard, Manassas, Va. 201 10-2209, USA, on Jun. 15, 2012. Vector B30VH having ATCC Accession No. PTA-121201 is a polynucleotide encoding the B30 heavy chain variable region, and vector B30VL having ATCC Accession No. PTA-121202 is a polynucleotide encoding the B30 light chain variable region. Vector R3BH1VH having ATCC Accession No. PTA-121203 is a polynucleotide encoding the R3BH1 heavy chain variable region, and vector R3BH1VL having ATCC Accession No. PTA-121204 is a polynucleotide encoding the R3BH1 light chain variable region. The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims. The contents of all figures and all references, patents and published patent applications cited throughout this application are expressly incorporated herein by reference.

### EXAMPLES

### Example 1

### Generation of Chicken Monoclonal Specific for BDNF and Generation of Chimeric Chicken-Human Monoclonal Specific for Human BDNF

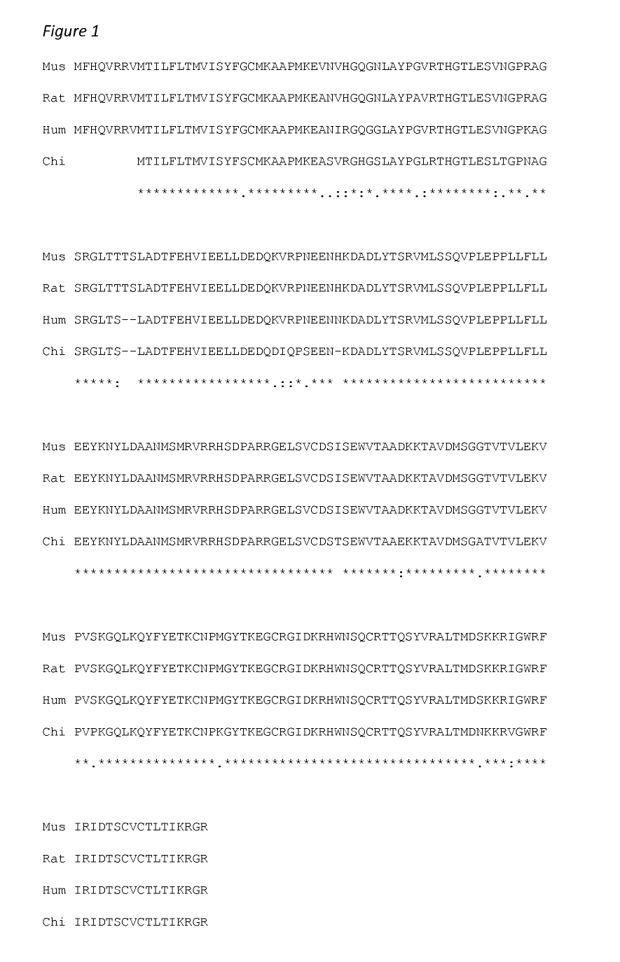

Because of the 100% sequence conservation between human, mouse and rat BDNF it was not a straightforward task to obtain a specific neutralizing anti-BDNF antibody by using the mouse monoclonal route. Sequence alignment of human BDNF with chicken BDNF highlighted a few key differences in amino acid sequence (FIG. 1) that permitted the use of chickens as an alternative immune host for BDNF and provided a possible method to obtain BDNF specific antibodies (Finlay et al, (2011) Methods Mol Biol 681; 87-101). In vivo immunization of chickens with the immunogen was coupled with in vitro phage display to derive high affinity, high specificity neutralizing anti-BDNF antibodies.

Immunisation was carried out as follows: three Leghorn/Brown chickens were immunized with a mixture of human BDNF and two unrelated antigens, human and mouse VEGF. The animals received four immunizations in total, at 20-day intervals, with 50 μg each protein per animal per immunization. Seven days after the fourth immunization, spleen and bone marrow were harvested from each animal for mRNA isolation.

Library generation was carried out as described in Finlay et al ((2011) Methods Mol Biol 681; 383-401) with the objective of generating high-affinity, high-specificity single-chain Fv antibodies from multi-antigen immunized chickens. Total RNA was extracted from each tissue, followed by cDNA generation using both oligo-dT and random hexamer primed first strand synthesis. RT-PCR was then performed to amplify the chicken variable gene repertoires (VH and VL) from that cDNA, and the PCR products were combined via overlapping PCR (SOE-PCR) to make a final single-chain Fv (scFv) construct. This scFv product was then cloned into the phage display vector pWRIL-10 to generate the library, named WyCH11, which had a total size of 5.6×10⁸ clones.

Library selection was carried out as follows. Production of phage using helper phage was carried out by standard methods. 200 ug of human BDNF were immobilized onto 10 mg of tosyl-functionalized paramagnetic beads (Dynabeads M-280, Invitrogen) overnight at 37 C in 100 mM NaPO₄+600 mM (NH₃)SO₄. The library (5×10¹² input phage) was blocked in PBS/3% nonfat dry milk/1% bovine serum albumin (BSA) and subjected to three rounds of binding to BDNF beads followed washing (5× in PBS/0.05% Tween-20 and 5× in PBS), elution with triethanolamine, infection of E. coli and reamplification. Prior to the third round, the library was deselected on BSA-loaded beads before BDNF bead selection.

Screening was carried out as follows. Soluble scFv expression was induced in 1-ml cultures of individual clones recovered from each round of selection, and periplasmic extracts of induced bacteria (“peripreps”) were tested for binding to human BDNF by ELISA and counterscreened for nonspecific binding to human serum albumin (HSA). In addition, scFv were tested for competition for binding of BDNF to the TrkB receptor in an ELISA, in which peripreps were mixed with human or mouse TrkB-Fc (10 nm and 20 nM, respectively) and then applied to immobilized BDNF. TrkB-Fc binding was detected with an HRP-conjugated anti human Fc reagent. Clones showing at least 50% reduction of TrkB-Fc binding and clear BDNF binding were sequenced. From a total of 400 clones screened, 59 isolates representing six unique sequences met the selection criteria. The six unique scFv were purified via Ni-NTA affinity chromatography and tested for their ability to inhibit BDNF-induced signaling in TrkB SHC1-U20S reporter cells (PathHunter, DiscoverX). Three clones were found to have clear inhibition in the cell-based assay, including R3BH1.

IgG generation was carried out as follows. Three neutralizing clones were converted to chicken-human chimeric antibodies by cloning the chicken VL and VH genes, respectively, in frame with human lambda constant region and human IgG1 heavy chain constant regions (including the L234A, L235A and G237A triple mutation to minimize effector function [Kasaian M T et al (2008) J Pharm Exp Ther 325: 882-892]. The clone R3BH1 was selected as a result of this process.

### Example 2

### Crystal Structure of BDNF-Homodimer in Complex with the Neutralizing Antibody Fragment R3BH1-Fab

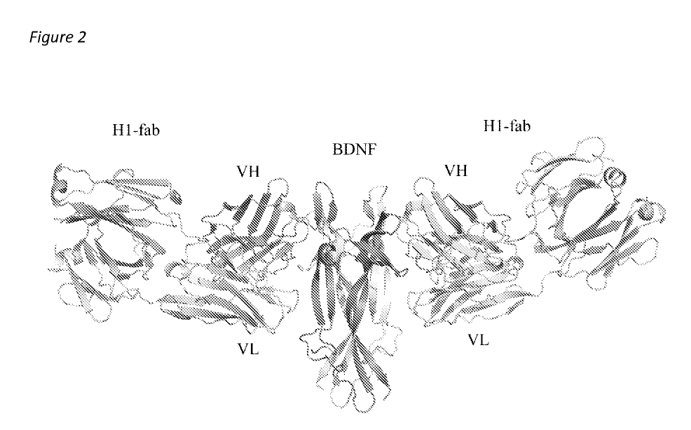

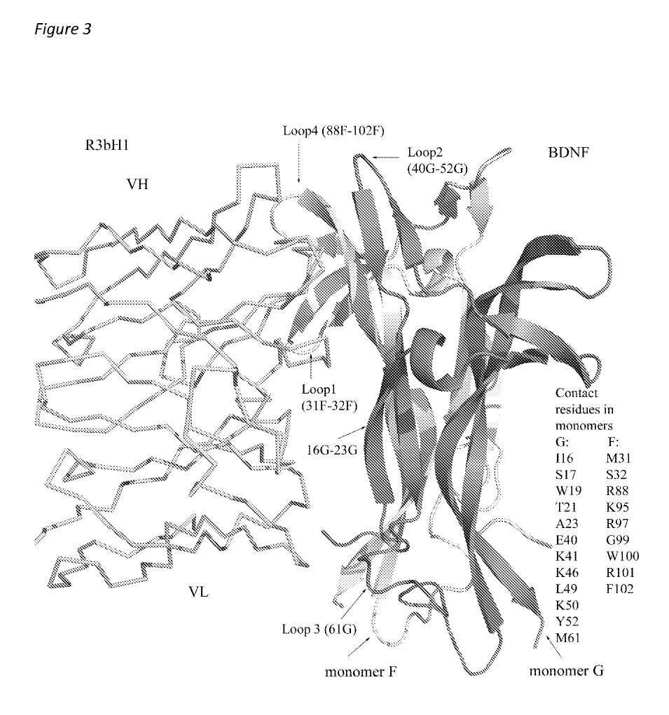

The co-crystal structure of BDNF-homodimer in complex with the neutralizing antibody fragment R3BH1-Fab has been determined. The crystal structure reveals two R3BH1-Fab molecules binding to the two symmetrical opposite sides of a single BDNF-homodimeric cytokine, as demonstrated by the cartoon diagram in FIG. 2. The C-termini of the R3BH1-Fab molecules are separated by about 150 Å, which imposes geometric constraints on observed stoichiometry and implies the 2:1 binding stoichiometry for R3BH1 and BDNF, with one BDNF molecule cross-linked by two spatially distant R3BH1 antibodies. The binding of R3BH1 to each of the opposite sides of BDNF creates two interacting surfaces and hence the two binding epitopes, numbers 1 and 2, on the BDNF surface. As both these binding surfaces involve similar interactions, details displayed in FIG. 3 refer to only one epitope, binding epitope number 1, that involves the antibody heavy (A) and light (B) chains, represented by the molecular ribbons in FIG. 3 and the BDNF-cytokine chains (F and G) represented by the cartoon diagram in FIG. 3. Details of the epitope residues contacted by the interaction of the partner R3BH1 that involves the antibody heavy (H) and light (L) chains are described in Table 6.

At each of the two interfaces, the BDNF residues involved in binding are contributed by both BDNF monomers, with 75% of the interactions coming from one monomer and the remaining 25% from the other monomer. A total of 21 residues from BDNF and 23 residues from R3BH1-Fab are involved in interactions at each interface as indicated by the fact that they are less than four angstroms (4 Å) apart and therefore considered “contact residues.” All CDR regions, except CDR-L3, are involved in interaction with BDNF, with the largest contribution coming from the heavy chain CDRs. Residues involved in interactions within 4 Å for both epitope 1 and 2, are listed in Table 5. All interactions within 4 Å, covering both antibody paratopes and binding epitopes 1 and 2, are listed in Table 6a to d. Table 6 a to d list in column 1 and 3: residue number, residue name, atom, atom type; in column 2 and 4:chain designation; in column 5: the interatomic distance between designated residue atoms in Å.

The observed crystal structure demonstrates that the neutralizing effect of R3BH1 is very likely due to direct competition of the antibody and the TrkB-receptor and p75NTR for the same binding determinants on BDNF. Table 7 and 8 provide a summary of R3BH1 paratope contact residues.

TABLE 5
Residues involved in interactions within 4
Å of R3BH1 for epitope 1 and 2.

| Epitope | | BDNF | BDNF |
|---|---|---|---|
| Number | BDNF Contact Residues | Domain | Chain |
| 1 | Met 31, Ser 32 | Loop 1 | F |
| 1 | Arg 88, Lys 95, Arg 97, Gly 99, Trp 100, | Loop 4 | F |
| | Arg 101, Phe 102 | | |
| 1 | Ile 16, Ser 17, Trp 19, Thr 21, Ala 23 | N- | G |
| | | terminal | |
| 1 | Glu 40, Lys 41, Lys 46, Leu 49, Lys 50, | Loop 2 | G |
| | Tyr 52 | | |
| 1 | Met 61 | Loop 3 | G |
| — | — | — | — |
| 2 | Met 31, Ser 32 | Loop 1 | G |
| 2 | Arg 88, Arg 97, Gly 99, Trp 100, Arg 101, | Loop 4 | G |
| | Phe 102 | | |
| 2 | Ile 16, Ser 17, Trp 19, Ala 23 | N- | F |
| | | terminal | |
| 2 | Glu 40, Lys 41, Leu 49, Lys 50, Tyr 52 | Loop 2 | F |
| 2 | Met 61 | Loop 3 | F |

TABLE 6a
R3BH1 Paratope [VH Chain A] contacts for
Epitope 1, 4 Å contact residues.

| Col- | | | | Col- |
|---|---|---|---|---|
| umn 1 | Col- | Col- | Col- | umn 5 |
| R3BH1 Paratope | umn2 | umn 3 | umn 4 | Dis- |
| VH (Chain A) | R3BH1 | Epitope number 1 | BDNF | tance |
| Atoms | Chain | BDNF Atoms | Chain | Å |
| 103(ILE)./CB [C] | A | 19(TRP)./CB [C] | G | 3.57 |
| 103(ILE)./CG1[C] | A | 19(TRP)./CB [C] | G | 3.75 |
| 103(ILE)./CB [C] | A | 19(TRP)./CG [C] | G | 3.54 |
| 103(ILE)./CG2[C] | A | 19(TRP)./CG [C] | G | 3.68 |
| 103(ILE)./O [O] | A | 19(TRP)./CD1[C] | G | 3.59 |
| 106(ASN)./OD1[O] | A | 19(TRP)./CD1[C] | G | 3.81 |
| 108(HIS)./CD2[C] | A | 19(TRP)./CD1[C] | G | 3.90 |
| 108(HIS)./NE2[N] | A | 19(TRP)./CD1[C] | G | 3.94 |
| 103(ILE)./CB [C] | A | 19(TRP)./CD1[C] | G | 3.64 |
| 103(ILE)./CG2[C] | A | 19(TRP)./CD1[C] | G | 3.64 |
| 103(ILE)./CG2[C] | A | 19(TRP)./CD2[C] | G | 3.69 |
| 106(ASN)./CG [C] | A | 19(TRP)./NE1[N] | G | 3.44 |
| 106(ASN)./OD1[O] | A | 19(TRP)./NE1[N] | G | 2.80 |
| 106(ASN)./ND2[N] | A | 19(TRP)./NE1[N] | G | 3.49 |
| 108(HIS)./CD2[C] | A | 19(TRP)./NE1[N] | G | 3.92 |
| 103(ILE)./CG2[C] | A | 19(TRP)./NE1[N] | G | 3.71 |
| 106(ASN)./OD1[O] | A | 19(TRP)./CE2[C] | G | 3.69 |
| 106(ASN)./ND2[N] | A | 19(TRP)./CE2[C] | G | 3.94 |
| 103(ILE)./CG2[C] | A | 19(TRP)./CE2[C] | G | 3.73 |
| 106(ASN)./CG [C] | A | 19(TRP)./CZ2[C] | G | 3.92 |
| 106(ASN)./OD1[O] | A | 19(TRP)./CZ2[C] | G | 3.95 |
| 106(ASN)./ND2[N] | A | 19(TRP)./CZ2[C] | G | 3.76 |
| 105(TRP)./CZ2[C] | A | 19(TRP)./CZ2[C] | G | 3.92 |
| 101(TYR)./CE2[C] | A | 21(THR)./CB [C] | G | 3.95 |
| 101(TYR)./CE2[C] | A | 21(THR)./OG1[O] | G | 3.97 |
| 101(TYR)./CE2[C] | A | 21(THR)./CG2[C] | G | 3.92 |
| 103(ILE)./CD1[C] | A | 21(THR)./CG2[C] | G | 3.90 |
| 101(TYR)./CE2[C] | A | 23(ALA)./CB [C] | G | 3.64 |
| 101(TYR)./CZ [C] | A | 23(ALA)./CB [C] | G | 3.85 |
| 101(TYR)./OH [O] | A | 23(ALA)./CB [C] | G | 3.55 |
| 101(TYR)./OH [O] | A | 40(GLU)./OE1[O] | G | 3.82 |
| 31(SER)./OG [O] | A | 41(LYS)./CE [C] | G | 3.85 |
| 31(SER)./OG [O] | A | 41(LYS)./NZ [N] | G | 3.63 |
| 74(ASN)./N [N] | A | 46(LYS)./NZ [N] | G | 3.86 |
| 73(ASP)./OD1[O] | A | 46(LYS)./NZ [N] | G | 3.93 |
| 75(GLY)./N [N] | A | 46(LYS)./NZ [N] | G | 3.43 |
| 53(ASP)./OD1[O] | A | 49(LEU)./CA [C] | G | 3.86 |
| 53(ASP)./OD1[O] | A | 49(LEU)./C [C] | G | 3.87 |
| 53(ASP)./OD1[O] | A | 49(LEU)./CD1[C] | G | 3.79 |
| 54(GLY)./N [N] | A | 49(LEU)./CD1[C] | G | 3.73 |
| 52(ASP)./OD2[O] | A | 49(LEU)./CD1[C] | G | 3.90 |
| 54(GLY)./CA [C] | A | 49(LEU)./CD1[C] | G | 3.49 |
| 53(ASP)./CG [C] | A | 50(LYS)./N [N] | G | 3.86 |
| 53(ASP)./OD1[O] | A | 50(LYS)./N [N] | G | 2.96 |
| 53(ASP)./OD1[O] | A | 50(LYS)./CA [C] | G | 3.80 |
| 53(ASP)./CG [C] | A | 50(LYS)./CB [C] | G | 3.65 |
| 53(ASP)./OD2[O] | A | 50(LYS)./CB [C] | G | 3.45 |
| 53(ASP)./OD1[O] | A | 50(LYS)./CB [C] | G | 3.42 |
| 101(TYR)./CZ [C] | A | 50(LYS)./CG [C] | G | 3.99 |
| 101(TYR)./OH [O] | A | 50(LYS)./CG [C] | G | 3.64 |
| 31(SER)./O [O] | A | 50(LYS)./CD [C] | G | 3.67 |
| 101(TYR)./CE1[C] | A | 50(LYS)./CD [C] | G | 3.53 |
| 101(TYR)./CZ [C] | A | 50(LYS)./CD [C] | G | 3.49 |
| 101(TYR)./OH [O] | A | 50(LYS)./CD [C] | G | 3.62 |
| 31(SER)./O [O] | A | 50(LYS)./CE [C] | G | 3.66 |
| 101(TYR)./O [O] | A | 50(LYS)./CE [C] | G | 3.29 |
| 101(TYR)./CE1[C] | A | 50(LYS)./CE [C] | G | 3.97 |
| 101(TYR)./CE2[C] | A | 50(LYS)./CE [C] | G | 3.93 |
| 101(TYR)./CZ [C] | A | 50(LYS)./CE [C] | G | 3.86 |
| 103(ILE)./CD1[C] | A | 50(LYS)./CE [C] | G | 3.66 |
| 31(SER)./O [O] | A | 50(LYS)./NZ [N] | G | 2.57 |
| 101(TYR)./O [O] | A | 50(LYS)./NZ [N] | G | 2.79 |
| 101(TYR)./C [C] | A | 50(LYS)./NZ [N] | G | 3.74 |
| 101(TYR)./CD1[C] | A | 50(LYS)./NZ [N] | G | 3.95 |
| 31(SER)./C [C] | A | 50(LYS)./NZ [N] | G | 3.70 |
| 103(ILE)./CG1[C] | A | 52(TYR)./CG [C] | G | 3.57 |
| 103(ILE)./CD1[C] | A | 52(TYR)./CG [C] | G | 3.57 |
| 103(ILE)./CG1[C] | A | 52(TYR)./CD1[C] | G | 3.98 |
| 103(ILE)./CD1[C] | A | 52(TYR)./CD1[C] | G | 3.43 |
| 103(ILE)./CG1[C] | A | 52(TYR)./CD2[C] | G | 3.44 |
| 103(ILE)./CD1[C] | A | 52(TYR)./CD2[C] | G | 3.91 |
| 103(ILE)./CD1[C] | A | 52(TYR)./CE1[C] | G | 3.67 |
| 103(ILE)./CG1[C] | A | 52(TYR)./CE2[C] | G | 3.74 |
| 103(ILE)./CG2[C] | A | 52(TYR)./CE2[C] | G | 3.47 |
| 103(ILE)./CG2[C] | A | 52(TYR)./CZ [C] | G | 3.75 |
| 105(TRP)./NE1[N] | A | 52(TYR)./CZ [C] | G | 3.91 |
| 103(ILE)./CD1[C] | A | 52(TYR)./CZ [C] | G | 3.99 |
| 103(ILE)./CG2[C] | A | 52(TYR)./OH [O] | G | 3.92 |
| 105(TRP)./CD1[C] | A | 52(TYR)./OH [O] | G | 3.56 |
| 105(TRP)./NE1[N] | A | 52(TYR)./OH [O] | G | 2.90 |
| 52(ASP)./CB [C] | A | 52(TYR)./OH [O] | G | 4.00 |
| 105(TRP)./CE2[C] | A | 52(TYR)./OH [O] | G | 3.96 |
| /1/A/59(TYR)./CE1[C] | A | 31(MET)./C [C] | F | 3.84 |
| 59(TYR)./CE1[C] | A | 31(MET)./O [O] | F | 3.50 |
| 106(ASN)./CG [C] | A | 31(MET)./SD [S] | F | 3.93 |
| 106(ASN)./ND2[N] | A | 31(MET)./SD [S] | F | 3.28 |
| 106(ASN)./CB [C] | A | 31(MET)./SD [S] | F | 3.72 |
| 59(TYR)./CD1[C] | A | 32(SER)./OG [O] | F | 3.46 |
| 59(TYR)./CE1[C] | A | 32(SER)./OG [O] | F | 3.96 |
| 58(THR)./O [O] | A | 32(SER)./OG [O] | F | 3.26 |
| 52(ASP)./OD2[O] | A | 88(ARG)./NH2[N] | F | 3.37 |
| 69(THR)./OG1[O] | A | 95(LYS)./NZ [N] | F | 3.96 |
| 69(THR)./CG2[C] | A | 95(LYS)./NZ [N] | F | 3.88 |
| 56(SER)./OG [O] | A | 97(ARG)./CD [C] | F | 3.83 |
| 55(GLY)./O [O] | A | 97(ARG)./NE [N] | F | 3.66 |
| 56(SER)./CA [C] | A | 97(ARG)./NE [N] | F | 3.67 |
| 56(SER)./CB [C] | A | 97(ARG)./NE [N] | F | 3.65 |
| 56(SER)./OG [O] | A | 97(ARG)./NE [N] | F | 2.77 |
| 55(GLY)./C [C] | A | 97(ARG)./CZ [C] | F | 3.84 |
| 55(GLY)./O [O] | A | 97(ARG)./CZ [C] | F | 3.43 |
| 56(SER)./OG [O] | A | 97(ARG)./CZ [C] | F | 3.42 |
| 55(GLY)./O [O] | A | 97(ARG)./NH1[N] | F | 3.97 |
| 55(GLY)./CA [C] | A | 97(ARG)./NH2[N] | F | 3.83 |
| 55(GLY)./C [C] | A | 97(ARG)./NH2[N] | F | 3.36 |
| 55(GLY)./O [O] | A | 97(ARG)./NH2[N] | F | 3.36 |
| 56(SER)./N [N] | A | 97(ARG)./NH2[N] | F | 3.72 |
| 54(GLY)./O [O] | A | 97(ARG)./NH2[N] | F | 3.33 |
| 56(SER)./OG [O] | A | 97(ARG)./NH2[N] | F | 3.22 |
| 56(SER)./OG [O] | A | 99(GLY)./CA [C] | F | 3.59 |
| 56(SER)./CB [C] | A | 100(TRP)./O [O] | F | 3.74 |
| 57(ASP)./CG [C] | A | 101(ARG)./CA [C] | F | 3.85 |
| 57(ASP)./OD1[O] | A | 101(ARG)./CA [C] | F | 3.97 |
| 57(ASP)./OD2[O] | A | 101(ARG)./CA [C] | F | 3.34 |
| 57(ASP)./OD2[O] | A | 101(ARG)./C [C] | F | 3.63 |
| 57(ASP)./OD1[O] | A | 101(ARG)./CB [C] | F | 3.77 |
| 57(ASP)./OD1[O] | A | 101(ARG)./CG [C] | F | 3.78 |
| 56(SER)./O [O] | A | 101(ARG)./CG [C] | F | 3.75 |
| 56(SER)./O [O] | A | 101(ARG)./CD [C] | F | 3.84 |
| 56(SER)./O [O] | A | 101(ARG)./NE [N] | F | 3.19 |
| 56(SER)./O [O] | A | 101(ARG)./CZ [C] | F | 3.97 |
| 57(ASP)./CG [C] | A | 102(PHE)./N [N] | F | 3.88 |
| 57(ASP)./OD2[O] | A | 102(PHE)./N [N] | F | 2.97 |
| 57(ASP)./OD2[O] | A | 102(PHE)./CB [C] | F | 3.98 |
| 57(ASP)./OD2[O] | A | 102(PHE)./CD2[C] | F | 3.51 |

TABLE 6b
R3BH1 Paratope VL [Chain A] contacts for
Epitope 1, 4 Å contact residues.

| Col- | | | | Col- |
|---|---|---|---|---|
| umn 1 | Col- | Col- | Col- | umn 5 |
| R3BH1 Paratope | umn 2 | umn 3 | umn 4 | Dis- |
| VL (chain B) | R3BH1 | Epitope number 1 | BDNF | tance |
| Atoms | Chain | BDNF Atoms | Chain | Å |
| 25(SER)./O [O] | B | 16(ILE)./CD1[C] | G | 3.98 |
| 26(GLY)./CA [C] | B | 16(ILE)./CD1[C] | G | 3.31 |
| 26(GLY)./C [C] | B | 16(ILE)./CD1[C] | G | 3.59 |
| 26(GLY)./O [O] | B | 16(ILE)./CD1[C] | G | 3.23 |
| 27(TYR)./OH [O] | B | 17(SER)./N [N] | G | 3.81 |
| 27(TYR)./OH [O] | B | 17(SER)./C [C] | G | 3.72 |
| 27(TYR)./CE2[C] | B | 17(SER)./O [O] | G | 3.28 |
| 27(TYR)./CZ [C] | B | 17(SER)./O [O] | G | 3.43 |
| 27(TYR)./OH [O] | B | 17(SER)./O [O] | G | 2.89 |
| 65(GLY)./N [N] | B | 61(MET)./SD [S] | G | 3.95 |
| 65(GLY)./CA [C] | B | 61(MET)./SD [S] | G | 3.64 |
| 63(LYS)./NZ [N] | B | 61(MET)./CE [C] | G | 3.48 |

TABLE 6c
R3BH1 Paratope VH [Chain H] contacts for
Epitope 2, 4 Å contact residues.

| Col- | | | | Col- |
|---|---|---|---|---|
| umn 1 | Col- | Col- | Col- | umn 5 |
| R3BH1 Paratope | umn 2 | umn 3 | umn 4 | Dis- |
| VH (Chain H) | R3BH1 | Epitope number 2 | BDNF | tance |
| Atoms | Chain | BDNF Atoms | Chain | Å |
| 59(TYR)./CE1[C] | H | 31(MET)./C [C] | G | 3.75 |
| 59(TYR)./CE1[C] | H | 31(MET)./O [O] | G | 3.33 |
| 106(ASN)./ND2[N] | H | 31(MET)./SD [S] | G | 3.46 |
| 106(ASN)./CB [C] | H | 31(MET)./SD [S] | G | 3.69 |
| 65(LYS)./NZ [N] | H | 32(SER)./O [O] | G | 3.25 |
| 58(THR)./O [O] | H | 32(SER)./OG [O] | G | 2.79 |
| 58(THR)./C [C] | H | 32(SER)./OG [O] | G | 3.67 |
| 59(TYR)./CA [C] | H | 32(SER)./OG [O] | G | 3.60 |
| 59(TYR)./CD1[C] | H | 32(SER)./OG [O] | G | 3.63 |
| 52(ASP)./OD2[O] | H | 88(ARG)./NH2[N] | G | 3.47 |
| 56(SER)./OG [O] | H | 97(ARG)./CD [C] | G | 3.91 |
| 56(SER)./CB [C] | H | 97(ARG)./NE [N] | G | 3.96 |
| 56(SER)./CA [C] | H | 97(ARG)./NE [N] | G | 3.95 |
| 56(SER)./OG [O] | H | 97(ARG)./NE [N] | G | 2.99 |
| 55(GLY)./O [O] | H | 97(ARG)./NE [N] | G | 3.62 |
| 56(SER)./OG [O] | H | 97(ARG)./CZ [C] | G | 3.76 |
| 55(GLY)./C [C] | H | 97(ARG)./CZ [C] | G | 3.95 |
| 55(GLY)./O [O] | H | 97(ARG)./CZ [C] | G | 3.49 |
| 56(SER)./OG [O] | H | 97(ARG)./NH2[N] | G | 3.66 |
| 55(GLY)./C [C] | H | 97(ARG)./NH2[N] | G | 3.59 |
| 55(GLY)./O [O] | H | 97(ARG)./NH2[N] | G | 3.52 |
| 54(GLY)./O [O] | H | 97(ARG)./NH2[N] | G | 3.52 |
| 55(GLY)./CA [C] | H | 97(ARG)./NH2[N] | G | 3.90 |
| 56(SER)./CB [C] | H | 99(GLY)./CA [C] | G | 3.97 |
| 56(SER)./OG [O] | H | 99(GLY)./CA [C] | G | 3.43 |
| 56(SER)./CB [C] | H | 100(TRP)./O [O] | G | 3.63 |
| 57(ASP)./OD2[O] | H | 101(ARG)./CA [C] | G | 3.34 |
| 57(ASP)./CG [C] | H | 101(ARG)./CA [C] | G | 3.77 |
| 57(ASP)./OD1[O] | H | 101(ARG)./CA [C] | G | 3.87 |
| 57(ASP)./OD2[O] | H | 101(ARG)./C [C] | G | 3.54 |
| 57(ASP)./OD1[O] | H | 101(ARG)./CB [C] | G | 3.70 |
| 57(ASP)./OD1[O] | H | 101(ARG)./CG [C] | G | 3.88 |
| 56(SER)./O [O] | H | 101(ARG)./CG [C] | G | 3.48 |
| 56(SER)./O [O] | H | 101(ARG)./CD [C] | G | 3.52 |
| 56(SER)./CB [C] | H | 101(ARG)./NE [N] | G | 3.99 |
| 56(SER)./C [C] | H | 101(ARG)./NE [N] | G | 3.95 |
| 56(SER)./O [O] | H | 101(ARG)./NE [N] | G | 2.99 |
| 56(SER)./O [O] | H | 101(ARG)./CZ [C] | G | 3.90 |
| 56(SER)./CB [C] | H | 101(ARG)./NH2[N] | G | 3.98 |
| 57(ASP)./OD2[O] | H | 102(PHE)./N [N] | G | 2.84 |
| 57(ASP)./CG [C] | H | 102(PHE)./N [N] | G | 3.69 |
| 57(ASP)./OD1[O] | H | 102(PHE)./N [N] | G | 3.81 |
| 57(ASP)./OD2[O] | H | 102(PHE)./CA [C] | G | 3.88 |
| 57(ASP)./OD2[O] | H | 102(PHE)./CB [C] | G | 3.81 |
| 57(ASP)./OD2[O] | H | 102(PHE)./CD2[C] | G | 3.35 |
| 103(ILE)./CB [C] | H | 19(TRP)./CB [C] | F | 3.71 |
| 103(ILE)./CG1[C] | H | 19(TRP)./CB [C] | F | 3.83 |
| 103(ILE)./CG2[C] | H | 19(TRP)./CG [C] | F | 3.73 |
| 103(ILE)./CB [C] | H | 19(TRP)./CG [C] | F | 3.64 |
| 103(ILE)./CG2[C] | H | 19(TRP)./CD1[C] | F | 3.64 |
| 106(ASN)./OD1[O] | H | 19(TRP)./CD1[C] | F | 3.77 |
| 103(ILE)./CB [C] | H | 19(TRP)./CD1[C] | F | 3.66 |
| 103(ILE)./O [O] | H | 19(TRP)./CD1[C] | F | 3.74 |
| 103(ILE)./CG2[C] | H | 19(TRP)./CD2[C] | F | 3.83 |
| 106(ASN)./CG [C] | H | 19(TRP)./NE1[N] | F | 3.35 |
| 106(ASN)./ND2[N] | H | 19(TRP)./NE1[N] | F | 3.43 |
| 103(ILE)./CG2[C] | H | 19(TRP)./NE1[N] | F | 3.73 |
| 106(ASN)./OD1[O] | H | 19(TRP)./NE1[N] | F | 2.79 |
| 106(ASN)./CG [C] | H | 19(TRP)./CE2[C] | F | 3.98 |
| 106(ASN)./ND2[N] | H | 19(TRP)./CE2[C] | F | 3.91 |
| 103(ILE)./CG2[C] | H | 19(TRP)./CE2[C] | F | 3.83 |
| 106(ASN)./OD1[O] | H | 19(TRP)./CE2[C] | F | 3.72 |
| 106(ASN)./CG [C] | H | 19(TRP)./CZ2[C] | F | 3.91 |
| 106(ASN)./ND2[N] | H | 19(TRP)./CZ2[C] | F | 3.80 |
| 105(TRP)./CZ2[C] | H | 19(TRP)./CZ2[C] | F | 3.79 |
| 105(TRP)./CZ2[C] | H | 19(TRP)./CH2[C] | F | 3.95 |
| 101(TYR)./OH [O] | H | 23(ALA)./CB [C] | F | 3.85 |
| 101(TYR)./OH [O] | H | 40(GLU)./CG [C] | F | 3.41 |
| 101(TYR)./OH [O] | H | 40(GLU)./CD [C] | F | 3.39 |
| 101(TYR)./CE1[C] | H | 40(GLU)./OE2[O] | F | 3.61 |
| 101(TYR)./CZ [C] | H | 40(GLU)./OE2[O] | F | 3.40 |
| 101(TYR)./OH [O] | H | 40(GLU)./OE2[O] | F | 2.61 |
| 31(SER)./OG [O] | H | 41(LYS)./NZ [N] | F | 3.05 |
| 53(ASP)./OD1[O] | H | 49(LEU)./CA [C] | F | 3.89 |
| 52(ASP)./OD2[O] | H | 49(LEU)./CD1[C] | F | 3.86 |
| 53(ASP)./OD1[O] | H | 49(LEU)./CD1[C] | F | 3.62 |
| 54(GLY)./N [N] | H | 49(LEU)./CD1[C] | F | 3.72 |
| 54(GLY)./CA [C] | H | 49(LEU)./CD1[C] | F | 3.50 |
| 53(ASP)./OD1[O] | H | 50(LYS)./N [N] | F | 3.21 |
| 53(ASP)./CG [C] | H | 50(LYS)./CB [C] | F | 3.96 |
| 53(ASP)./OD1[O] | H | 50(LYS)./CB [C] | F | 3.76 |
| 53(ASP)./OD2[O] | H | 50(LYS)./CB [C] | F | 3.68 |
| 101(TYR)./OH [O] | H | 50(LYS)./CG [C] | F | 3.80 |
| 53(ASP)./CG [C] | H | 50(LYS)./CD [C] | F | 3.84 |
| 53(ASP)./OD1[O] | H | 50(LYS)./CD [C] | F | 3.99 |
| 53(ASP)./OD2[O] | H | 50(LYS)./CD [C] | F | 3.85 |
| 101(TYR)./CE1[C] | H | 50(LYS)./CD [C] | F | 4.00 |
| 31(SER)./O [O] | H | 50(LYS)./CD [C] | F | 3.35 |
| 101(TYR)./CE2[C] | H | 50(LYS)./CE [C] | F | 4.00 |
| 101(TYR)./O [O] | H | 50(LYS)./CE [C] | F | 3.40 |
| 103(ILE)./CD1[C] | H | 50(LYS)./CE [C] | F | 3.84 |
| 101(TYR)./CE1[C] | H | 50(LYS)./CE [C] | F | 3.73 |
| 101(TYR)./CZ [C] | H | 50(LYS)./CE [C] | F | 3.77 |
| 31(SER)./O [O] | H | 50(LYS)./CE [C] | F | 3.54 |
| 101(TYR)./CD1[C] | H | 50(LYS)./CE [C] | F | 3.95 |
| 101(TYR)./O [O] | H | 50(LYS)./NZ [N] | F | 2.78 |
| 103(ILE)./CD1[C] | H | 50(LYS)./NZ [N] | F | 3.97 |
| 31(SER)./C [C] | H | 50(LYS)./NZ [N] | F | 3.91 |
| 31(SER)./O [O] | H | 50(LYS)./NZ [N] | F | 2.81 |
| 101(TYR)./C [C] | H | 50(LYS)./NZ [N] | F | 3.77 |
| 103(ILE)./CG1[C] | H | 52(TYR)./CG [C] | F | 3.76 |
| 103(ILE)./CD1[C] | H | 52(TYR)./CG [C] | F | 3.81 |
| 103(ILE)./CD1[C] | H | 52(TYR)./CD1[C] | F | 3.55 |
| 103(ILE)./CG1[C] | H | 52(TYR)./CD2[C] | F | 3.69 |
| 103(ILE)./CD1[C] | H | 52(TYR)./CE1[C] | F | 3.70 |
| 103(ILE)./CG2[C] | H | 52(TYR)./CE2[C] | F | 3.61 |
| 103(ILE)./CG1[C] | H | 52(TYR)./CE2[C] | F | 3.97 |
| 103(ILE)./CG2[C] | H | 52(TYR)./CZ [C] | F | 3.76 |
| 105(TRP)./NE1[N] | H | 52(TYR)./CZ [C] | F | 3.92 |
| 103(ILE)./CG2[C] | H | 52(TYR)./OH [O] | F | 3.85 |
| 105(TRP)./CD1[C] | H | 52(TYR)./OH [O] | F | 3.57 |
| 105(TRP)./NE1[N] | H | 52(TYR)./OH [O] | F | 2.86 |
| 105(TRP)./CE2[C] | H | 52(TYR)./OH [O] | F | 3.91 |

TABLE 6d
R3BH1 Paratope VL [Chain L] contacts for Epitope 2, 4 Å contact
residues.

| | Column | | | |
|---|---|---|---|---|
| Column 1 | 2 | Column 3 | Column 4 | |
| R3BH1 Paratope | R3BH1 | Epitope number 2 | BDNF | Column 5 |
| VL - Atoms | Chain | BDNF - Atoms | Chain | Distance Å |
| 27(TYR)./OH [O] | L | 16(ILE)./CG1[C] | F | 3.86 |
| 25(SER)./O [O] | L | 16(ILE)./CD1[C] | F | 3.95 |
| 27(TYR)./OH [O] | L | 17(SER)./N [N] | F | 3.95 |
| 27(TYR)./OH [O] | L | 17(SER)./C [C] | F | 3.78 |
| 27(TYR)./CE2[C] | L | 17(SER)./O [O] | F | 3.62 |
| 27(TYR)./CZ [C] | L | 17(SER)./O [O] | F | 3.64 |
| 27(TYR)./OH [O] | L | 17(SER)./O [O] | F | 2.80 |
| 26(GLY)./CA [C] | L | 61(MET)./CE [C] | F | 3.83 |
| 26(GLY)./O [O] | L | 61(MET)./CE [C] | F | 3.59 |

TABLE 7
R3BH1 Paratope contact residues

| Region | Residue | Domain |
|---|---|---|
| VH | Ser 31 | CDR-H1 |
| VH | Asp 52, Asp 53, Gly 54, Gly 55, Ser | CDR-H2 |
| | 56, Asp 57, Thr 58 | |
| VH | Tyr 59, Thr 69, Asp 73, Asn 74, Gly 75 | Framework |
| VH | Tyr 101, Ile 103, Trp 105, Asn 106, His | CDR-H3 |
| | 108 | |
| VL | Ser 25, Gly 26, Tyr 27 | CDR-L1 |
| VL | Lys 63, Gly 65 | CDR-L2 |

TABLE 8
R3BH1 Paratope contact residues and related epitope contact

| | R3BH1 | BDNF | |
|---|---|---|---|
| R3BH1 | Contact | Contact | BDNF |
| Domain | Residue | Residue | Chain |
| CDR-H1 | SER 31 | LYS 41 | G |
| CDR-H1 | SER 31 | LYS 50 | G |
| CDR-H2 | ASP 52 | LEU 49 | G |
| CDR-H2 | ASP 52 | TYR 52 | G |
| CDR-H2 | ASP 52 | ARG 88 | F |
| CDR-H2 | ASP 53 | LEU 49 | G |
| CDR-H2 | ASP 53 | LYS 50 | G |
| CDR-H2 | GLY 54 | LEU 49 | G |
| CDR-H2 | GLY 54 | ARG 97 | F |
| CDR-H2 | GLY 55 | ARG 97 | F |
| CDR-H2 | SER 56 | ARG 97 | F |
| CDR-H2 | SER 56 | GLY 99 | F |
| CDR-H2 | SER 56 | TRP 100 | F |
| CDR-H2 | SER 56 | ARG 101 | F |
| CDR-H2 | ASP 57 | ARG 101 | F |
| CDR-H2 | ASP 57 | PHE 102 | F |
| CDR-H2 | THR 58 | SER 32 | F |
| FW | TYR 59 | MET 31 | F |
| FW | TYR 59 | SER 32 | F |
| FW | THR 69 | LYS 95 | F |
| FW | ASP 73 | LYS 46 | G |
| FW | ASN 74 | LYS 46 | G |
| FW | GLY 75 | LYS 46 | G |
| CDR-H3 | TYR 101 | THR 21 | G |
| CDR-H3 | TYR 101 | ALA 23 | G |
| CDR-H3 | TYR 101 | GLU 40 | G |
| CDR-H3 | TYR 101 | LYS 50 | G |
| CDR-H3 | ILE 103 | TRP 19 | G |
| CDR-H3 | ILE 103 | THR 21 | G |
| CDR-H3 | ILE 103 | LYS 50 | G |
| CDR-H3 | ILE 103 | TYR 52 | G |
| CDR-H3 | TRP 105 | TRP 19 | G |
| CDR-H3 | TRP 105 | TYR 52 | G |
| CDR-H3 | ASN 106 | TRP 19 | G |
| CDR-H3 | ASN 106 | MET 31 | FG |
| CDR-H3 | HIS 108 | TRP 19 | G |
| CDR-L1 | SER 25 | ILE 16 | G |
| CDR-L1 | GLY 26 | ILE 16 | G |
| CDR-L1 | TYR 27 | SER 17 | G |
| CDR-L2 | LYS 63 | MET 61 | G |
| CDR-L2 | GLY 65 | MET 61 | G |

### Example 3

### Molecular Modelling of the Anti-BDNF R3BH1 Paratope and BDNF Epitope

Predicted important epitope and paratope residues from the R3BH1-BDNF crystal structure were determined using algorithms to detect the buried surface area and electrostatic contacts presented in the crystallographic model structure, these measures were combined with the mutability prediction from Discovery studio (Accelrys Software Inc., Discovery Studio Modeling Environment, Release 3.5, San Diego) governing amino acid acceptability at any given site in the binding interface. Key predicted residues that make direct contacts were determined excluding residues that might alter the binding affinity indirectly by stabilizing the structure. Five sets of predicted key paratope and epitope clusters are presented in Table 9 for which the epitope is defined using the crystal structure numbering and the paratope by Kabat numbering.

TABLE 9
R3BH1 Predicted key paratope and epitope residue combinations.

| Paratope | |
|---|---|
| and | |
| epitope | |
| group | |
| 1 | Epitope TRP 19, LYS 41, LYS 50, TYR 52, ARG 88, |
| | ARG 97, ARG 101 |
| | Paratope ASP 33, ASP52, ASP53, ASP57, TYR 101, ILEU |
| | 103, TRP 105, ASN 106, TYR 27 |
| 2 | Epitope ILE 16, MET 31, LEU 49, GLY 99, PHE 102 |
| | Paratope ASP 28, GLY 55, TYR 59 |
| 3 | Epitope THR 21, SER 32, SER 17, GLU 40, MET 61, ASP |
| | 30 Paratope GLY 54, SER 56, SER 31, THR 58, GLY26, |
| | ALA 93, SER 30 |
| 4 | Epitope ALA 23, GLN 48, TRP 100 |
| | Paratope ARG 72 SER 100 |
| 5 | Epitope ILEU 98, GLU 18, ASP 24, ARG 104 |
| | Paratope TYR 32, TYR 60, LYS 65, ASP 102, SER 104 |
| | HIS 108 SER 25, LYS LYS 63. GLY 65 |

### Example 4

### Anti-BDNF R3BH1 Binding to BDNF

Surface plasmon resonance (SPR) was used to characterize the binding kinetics of human BDNF to anti-BDNF chicken derived antibody R3B-H1. A low density of human BDNF was amine coupled onto a carboxymethylated dextran sensor chip surface (CM5) using a Biacore T200 instrument (GE Healthcare). After direct immobilization of antigen, three-fold serial dilutions of antibody R3B-H1 ranging from 243 nM to 9 nM were injected at a flow rate of 100 μl/min for a 47 sec association step and either a 300 or 4000 sec dissociation step. The human BDNF surface was regenerated with 3 pulses, 30 sec each, of 10 mM glycine pH 1.5 at a flow rate of 50 μL/min. The BDNF surface was then equilibrated with a single 30 sec pulse of HBS-EP+buffer (0.01 M HEPES, 0.15 M NaCl, 3 mM EDTA, and 0.05% v/v surfactant P20 pH 7.4) at a flow rate of 50 μL/min. All SPR experiments were performed at 37° C. and a data collection rate of 1 Hz with HBS-EP+ used as both the sample and running buffer. The resulting sensorgrams were double referenced with buffer injections and a non-derivatized flow cell surface. Rate constants were determined by applying a 1:1 Langmuir binding model using the T200 evaluation software v1.0 and the equation K_{D}=k_{d}/kₐ.

A concentration series of R3B-H1 was flowed over a BIAcore CM5 sensor chip with directly immobilized human BDNF. An association injection of 47 seconds was followed by dissociation steps of varying lengths. These data were fit to a 1:1 Langmuir binding model using Biacore T200 evaluation software v1.0 with the fit lines displayed in black. Sensorgrams shown are representative of data collected in triplicate across the 3 flow cells of a CM5 sensorchip.

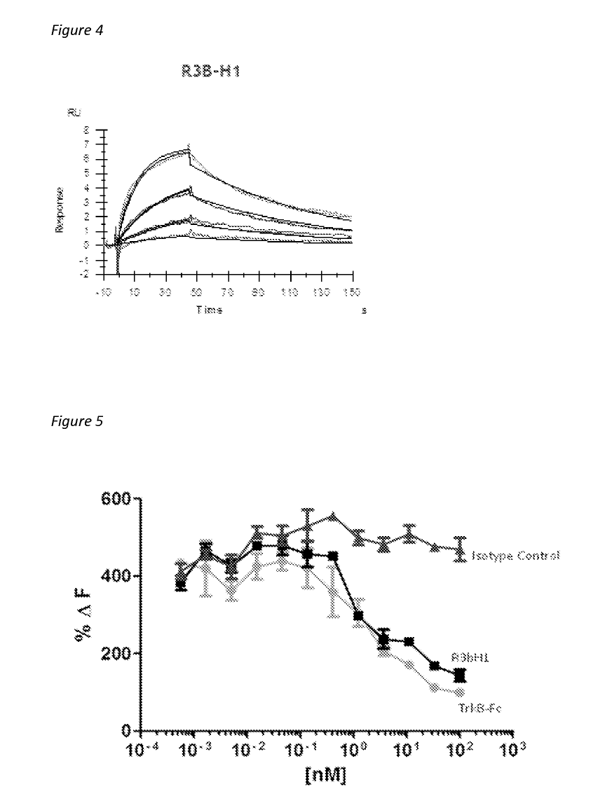

Surface plasmon resonance was used to determine the binding kinetics of chicken derived anti-BDNF antibody to the human BDNF antigen. Low densities of human BDNF were directly immobilized onto a BIAcore sensor chip to reduce non-specific binding of this antigen and to minimize avidity effects. For the evaluated antibodies, the range of antibody concentration flowed over the human BDNF prepared sensor chip surface provided dose dependent resonance unit (RU) responses. The dissociation phase for the highest concentration of antibody was extended to 4000 seconds to achieve a signal decay minimum of 5%. The kinetic rate constants were determined using a Langmuir 1:1 binding model and T200 evaluation software v1.0. The calculated K_{D} values are reported in the Table 10 below, the data trace is presented in FIG. 4.

TABLE 10
Binding Kinetics of Anti-BDNF R3B-H1 to Human BDNF

| | Human BDNF | | | |
|---|---|---|---|---|
| | ka (1/Ms) | | | |
| Antibody | 1 × E+05 | kd (1/s) 1 × E−02 | T_{1/2} (s) | KD (pM) |
| R3B-H1 | 3.03 ± 0.22 | 1.03 ± 0.08 | 68 | 34420 ± |
| | | | | 5431.7 (n = 3) |

### Example 5

### Anti-BDNF R3BH1 Inhibition of BDNF Binding to TrkB Receptor

A competition homogenous time-resolved fluorescence assay HTRF assay was established to screen for anti-BDNF antibodies that were capable of displacing BDNF bound TrkB receptor. Recombinant TrkB-Fc (R&D Systems) was labelled with europium cryptate using a cryptate labeling kit (CisBio) according to manufacturer's instructions. The final assay mixture consisted of 2.5 nM biotinylated human BDNF, 1/500 dilution of europium cryptate labeled TrkB-Fc, 1/2000 dilution of SA-XL665 (CisBio), and a dilution series of purified anti-BDNF antibody R3BH1 from 0-25 nM in a total reaction volume of 20 μl in 1× assay buffer [50 mM sodium phosphate, pH 7.5, 400 mM potassium fluoride, and 0.1% BSA (w/v)]. Reagents were added sequentially on the MiniTrak Liquid Handling Platform (Perkin-Elmer) into 384-well low volume black plates (Nunc). Reactions were allowed to proceed for 3 hours at room temperature and plates were subsequently read on the EnVision MultiLabel Plate Reader (Perkin-Elmer) with excitation at 340 nm and two emission readings at 615 nm (measuring input donor fluorescence from TrkB-Fc europium cryptate) and 665 nm (measuring output acceptor fluorescence from SAXL665). All readings were expressed as a ratio of fluorescence at 665/615 and data were plotted using Decision Site 8 (Spotfire) and Prism 5 software (GraphPad). The data in FIG. 5 demonstrate that the anti-BDNF R3BH1 molecule specifically binds BDNF and inhibits its interaction with the TrkB receptor with an IC₅₀ range of 0.1-0.5 nM

### Example 6

### Anti-BDNF R3BH1 Inhibition of BDNF Binding to p75NTR

The ability of anti-BDNF antibody R3BH1 to block binding of BDNF to p75NTR receptor was investigated using an SPR assay run on the BIAcore T200. Running conditions were as follows: buffer (HBS-P+1 mg/ml BSA) and flow rate 50 μl/min with a 50 second on-rate and 5 min-off rate analysis. Samples prepared and run comprised: (i) buffer alone; (ii) BDNF (20 nM) alone and (iii) BDNF (20 nM) and R3BH1 IgG or isotype control IgG (0.05-500 nM). All samples were incubated at RT for 30 mins prior to injection. 750 RU of p75NTR were immobilized on flow cells 2 and 3. 750 RU of p75NTR were immobilized on flow cell 1 and subsequently chemically inactivated for use as a reference control.

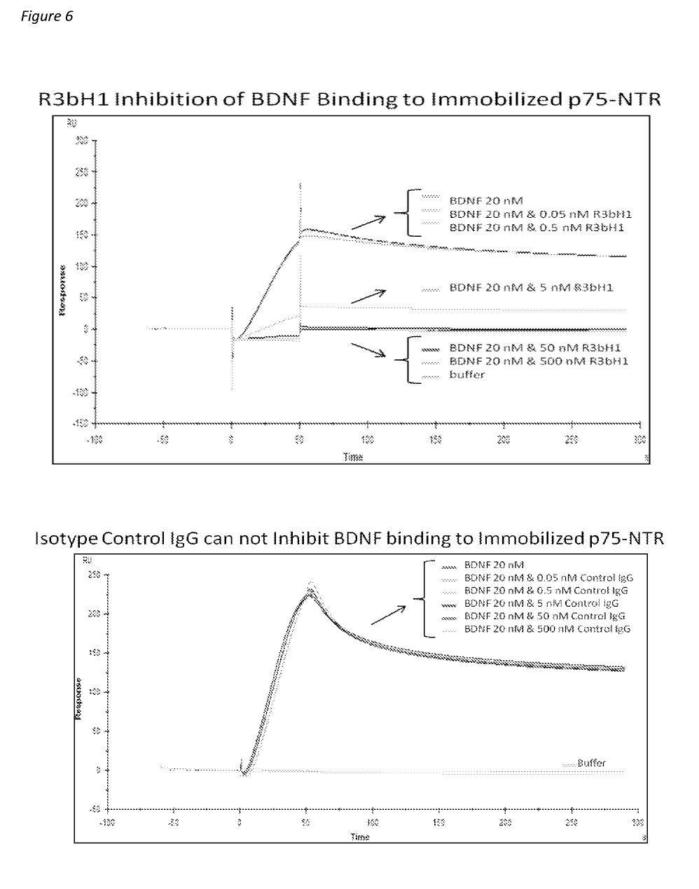

The data in FIG. 6 demonstrate that addition of 20 nM BDNF in the absence of IgG pre-incubation results in a signal of 150-200 RU. This signal is decreased in a dose-dependent manner when increasing concentrations of R3BH1 are added to 20 nM BDNF in a pre-incubation step. No decrease in signal is observed upon pre-incubation of BDNF with an isotype control IgG, even at 500 nM. The data show dose-dependent inhibition of BDNF-p75NTR interaction and shows that there is specificity of BDNF binding and functional activity of specific inhibition of BDNF p75NTR interaction.

### Example 7

### Selectivity of Anti-BDNF Antibody R3BH1:Cross-Reactivity with Other Related Neurotrophins and Similarly Positively Charged Chemokines

NUNC plates were coated with 1 μg/ml of a neurotrophin or chemokine in 1×PBS overnight. The proteins tested were: (i) recombinant hBDNF (positive control for binding) (ii) recombinant CXCL3 (iii) recombinant human CXCL9 (iv recombinant human CXCL10 (v) recombinant human CXCL13 (vi) recombinant human neurotrophin-3 (NT3), (vii) recombinant human neurotrophin-4 (NT4), (viii) recombinant human p75NTR, (ix) recombinant human β-NGF. After overnight incubation the plates were washed with PBS and blocked for 1 h in blocking buffer (3% skimmed milk/1% bovine serum albumin) in a volume of 200 μL. The panel of antibodies including R3BH1 and four commercially available anti-BDNF mouse monoclonal antibodies were titrated across the wells from 2000 to 1.25 nM and incubated for 1 h in blocking buffer (100 μL total volume). Plates were washed and a 1/5000 dilution of anti-IgG-HRP (horse radish peroxidase) in blocking buffer was added for 1 h (100 μL total volume). Plates were washed and developed by addition of 3,3′,5,5′-Tetramethylbenzidine (TMB) substrate and subsequently stopped with phosphoric acid.

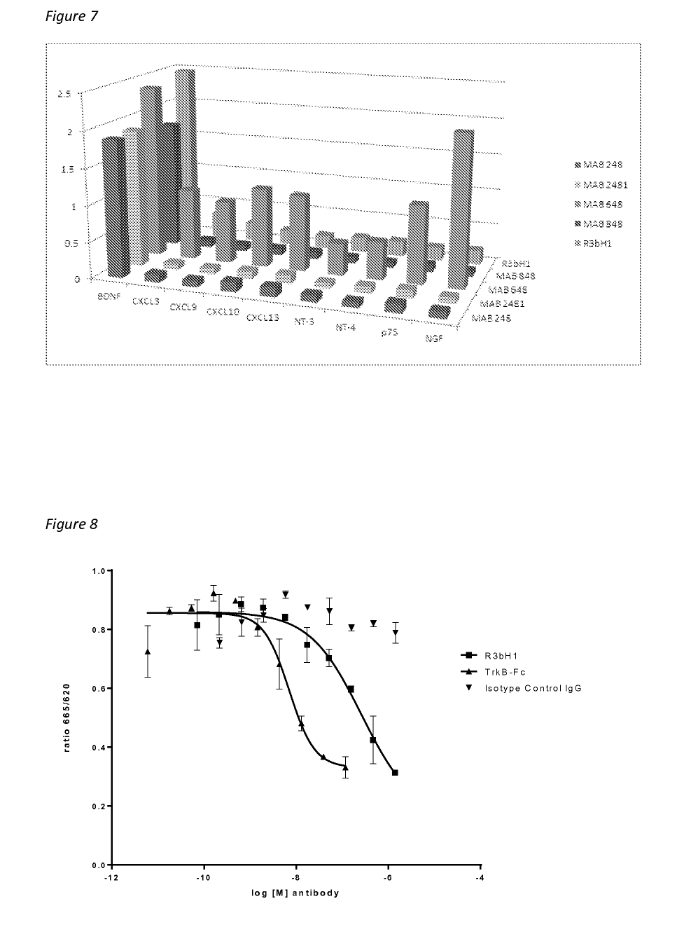

The data in FIG. 7 demonstrate that the R3BH1 specifically binds to BDNF and fails to recognise other neurotrophins such as NT-3, NT-4 and NGF. A panel of small, positively charged chemokines were included in the specificity analysis to ensure there were no non-specific charge-mediated interactions. The panel of commercially available anti-BDNF mouse monoclonal antibodies from R&D systems were also included for comparative purposes. This analysis indicated that mAb 648 from R&D systems [also denoted 37141] shows polyspecificity for multiple neurotrophins.

### Example 8

### Anti-BDNF Antibody R3BH1 Inhibits Activity of BDNF at the TrkB and p75NTR Receptors in TrkB/p75NTR Expressing Cells

The pERK (phospho-extracellular signal-regulated kinase) assay was used to demonstrate the effect of BDNF antibody R3BH1 on the functional activity of BDNF at TrkB receptors. Binding of BDNF to the TrkB receptor results in receptor dimerization and transphosphorylation of tyrosine residues, which creates docking sites for proteins that are involved in the downstream signalling events. Phosphorylated Erk can be detected following acute BDNF application and this can be quantified in the assay using two different specific monoclonal antibodies: a labelled anti-phospho-ERK antibody and a labelled anti-ERK antibody.

U2OS cells expressing TrkB+p75NTR (DiscoverX Corp.) were plated overnight in minimum essential medium (MEM; Life Technologies)+0.5% horse serum (Life Technologies). On the day of the assay, R3BH1, and commercial anti-BDNF antibodies were serial diluted 1:3 in phosphate buffered solution to create a 10 point concentration response curve. 10 ul of the serial diluted samples were then added to the cells and incubated for 1 h at 37 deg, before the addition of 10 ul of 1.8 nM BDNF (Peprotech) in PBS+0.25% BSA to each well (BDNF final assay concentration (FAC): 150 pM). The plate was incubated for 30 minutes at room temperature before media removal and the addition of Cellul'erk lysis buffer (Cisbio). The plate was then stored at −80 deg Celsius overnight. After thawing, lysates were transferred to a 384 well Proxiplate (Perkin Elmer) and Cellul'erk HTRF reagents added and incubated as per kit instructions before reading on an Envision plate reader (Perkin Elmer).

As shown in FIG. 8, presence of the anti-BDNF antibody R3BH1 inhibits BDNF mediated activation of the TrkB receptor (IC50 262 nM), and subsequent pERK activation in the cells. This confirms that R3BH1 is able to neutralise BDNF activity. None of the tested commercial antibodies produced dose dependent reductions of pERK activity in TrkB/p75NTR expressing U2OS cells.

TABLE 11
Commercial anti-BDNF antibodies profiled through cell based
assays of Examples 8 and 9

| | | | | mg/ml |
|---|---|---|---|---|
| | supplier and | species | | [working |
| name | cat number | raised in | Lot # | stock] |
| TrkB-Fc | Sigma T8694 | mouse | 070M1402 | 71 mg/ml |
| EPR1292 | Abcam | rabbit | YJ032313DS | |
| | ab108319 | | (both) | |
| | Origene | | | |
| | TA307523 | | | |
| EPR1293 | Abcam | rabbit | YJ101901CS | |
| | ab108383 | | (both) | |
| | Origene | | | |
| | TA307522 | | | |
| MM0109- | Abcam | mouse | GR26925-1 | 0.5 mg/ml |
| 3D44 | ab89155 | | | |
| msxghk- | Abcam | mouse | GR144200-1 | 1 mg/ml |
| BDNF | ab40934 | | | |
| 35928 | R&D MAB 248 | mouse | VH211202 | 1.7 mg/ml |
| 1B10 | Novus | mouse | DB111-1b10 | 1 mg/ml |
| | H00000627m02 | | | |
| 35928.11 | Novus NB120- | mouse | 041M1323 | 0.5 mg/ml |
| | 10505 | | | |
| | Abcam | | GR22266-5 | 0.5 mg/ml |
| | ab10505 | | | |
| | Sigma B5050 | | 041M1323 | 1 mg/ml |
| 37129 | R&D MAB848 | mouse | BBL1512091 | 1.7 mg/ml |
| | Sigma B9686 | | 098K0580 | 1 mg/ml |
| 35909 | R&D MAB2481 | mouse | JUD0211091 | 1.7 mg/ml |
| | Sigma B9561 | | 108K0422 | 1 mg/ml |
| 37141 | Sigma B9436 | mouse | 098K0575 | 1 mg/ml |
| | R&D MAB 648 | | BD10212041 | 1.7 mg/ml |

### Example 9

### Anti-BDNF Antibody R3BH1 Inhibits Activity of BDNF at TrkB Receptors in Recombinant TrkB/p75NTR Cells Using the PathHunter Assay

The PathHunter technology from DiscoverX utilizes Enzyme Fragment Complementation (EFC) to detect protein-protein interactions. In the TrkB/p75NTR expressing cells a small peptide epitope (ProLink) is expressed on the C-terminus of TrkB and co-expressed with an enzyme acceptor (EA) attached to a SH2 phospho-tyrosine binding domain. When the TrkB is activated by BDNF, receptor dimerisation and autophosphorylation occurs which subsequently recruits the SH2 domain to the activated receptor. The protein-protein interaction between the ProLink and EA generates an active beta-galactosidase enzyme, which can be detected using a chemiluminescent substrate. In the assay set up described here, TrkB activation will occur only from BDNF that has not been neutralized by the anti-BDNF antibodies and this can be used as an indirect measure of antibody functional activity.

U2OS cells expressing TrkB+p75NTR (DiscoverX Corp.) were plated into a 384 well TC plate in minimum essential medium (MEM; Life Technologies)+0.5% horse serum (Life Technologies) at 10,000 cells per well, 40 ul volume, and left in a 37 deg Celsius incubator overnight.

On the day of the assay, R3b-H1, TrkB-Fc, negative control (anti-tetanus IgG1) and commercial antibodies were serialised 1:3 in phosphate buffered solution to create a 12 point concentration response curve. 10 ul of the serialised samples were then added to the cells and incubated for 1 h at 37 deg. Following the 1 h incubation, 10 μl of 1.8 nM BDNF (Peprotech) in PBS+0.25% BSA was added to each well, BDNF final assay concentration (FAC): 300 pM, R3BH1 FAC: 5.63 uM-0.032 nM. The plate was incubated for 3 h at room temperature before the addition of 20 ul per well of PathHunter Detection reagent (DiscoverX Corp.) and the plate left at room temperature for 1 h before reading luminesence on an Envision plate reader (Perkin Elmer).

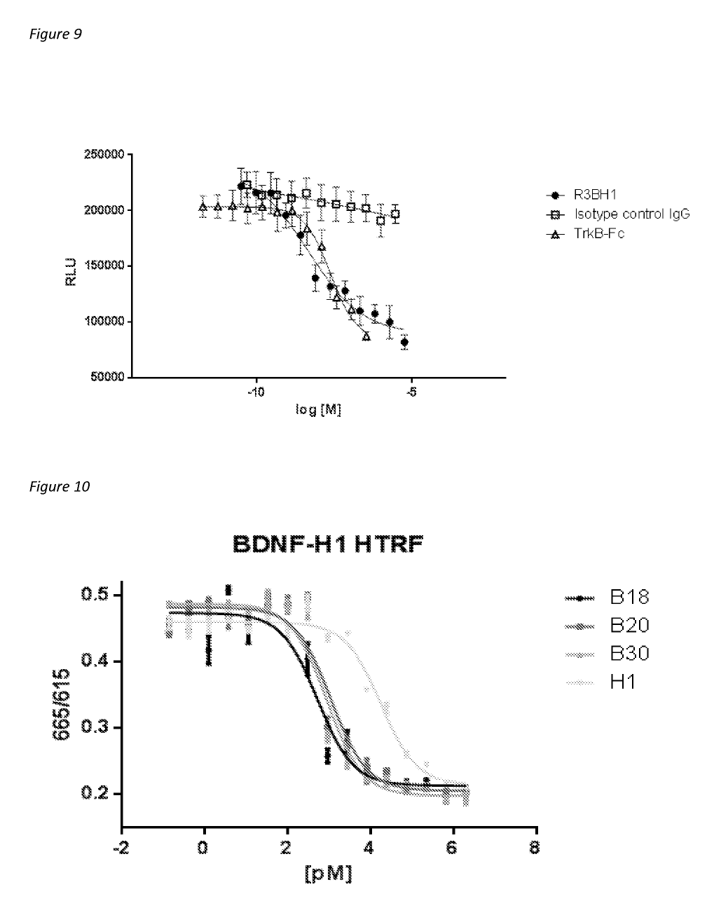

In FIG. 9, R3BH1 and TrkB-Fc are shown to display concentration dependent inhibition of BDNF activity at the TrkB receptor in U2OS cells, with IC5Os of 4.7 nM and 24 nM, respectively. With the exception of clone 37141 [Mab 648 mouse monoclonal], none of the commercial antibodies tested displayed neutralising activities in the assay. Note that clone 37141 [Mab 648] antibody was shown to display cross reactivity with multiple neurotrophins and also bind to similarly charged chemokines (Example 7). The negative control hIgG1 produced no inhibition of pTrkB activity.

### Example 10

### Humanization of Chicken-Human Chimeric Clone, R3BH1

R3BH1 was chosen for humanization (IgG1, lambda) based on its neutralization activity and favourable BDNF binding epitope. The overall humanization approach was to graft the chicken R3BH1 complementarity determining regions (CDRs) onto stable human acceptor frameworks. For humanization of chicken R3BH1 light chain variable region (VL), the CDRs as defined by Kabat were grafted onto the human germline acceptor framework DPL16. Similarly the chicken R3BH1 heavy chain variable regions (VH) CDRs were grafted onto the human germline acceptor framework DP-47. A single back-mutation, L46T, was required in the VL-FW1 region to retain the functionality of the parental chicken-human chimeric IgG. This has been previously described (Tsurishita et al (2004) J Immunol Methods 295; 9-19). The resulting humanized clone, H1, was shown to maintain affinity for BDNF and was functional in pERK and Pathfinder assays.

### Example 11

### Affinity Optimization of Humanized R3BH1 Clone

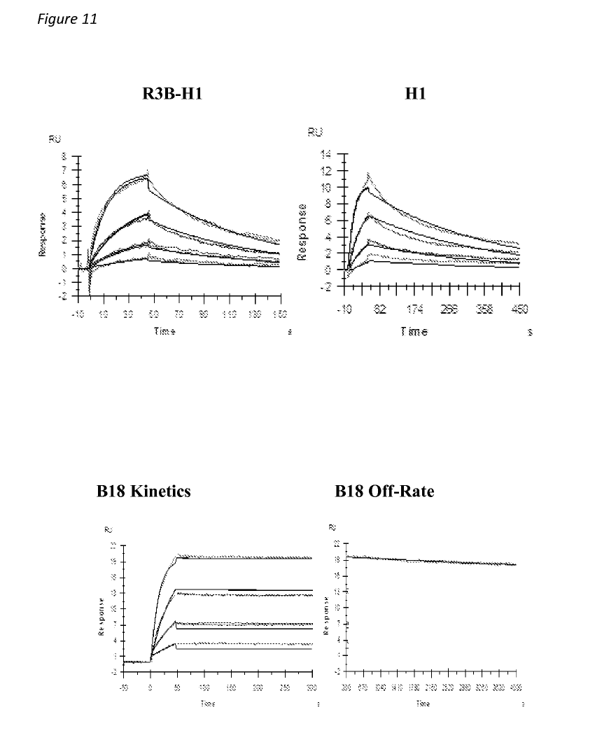

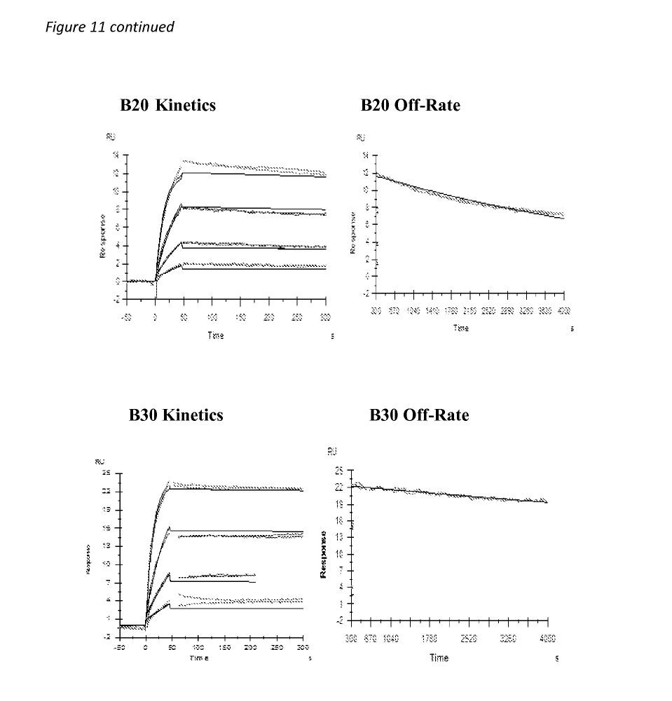

The humanized H1 clone was used as a template for affinity optimization. Two approaches were taken to optimization and in each case only 5 of the CDRs were targeted (namely VH-CDR1, VH-CDR2, VH-CDR3 & VL-CDR1, VL-CDR3). The first approach used soft randomization whereby each position in the named CDRs was targeted for mutagenesis with 50% wild-type amino acid/50% any other amino acid representation. The second approach was more tailored and involved specific mutagenesis of paratope residues defined by the co-crystal structure described in FIG. 2. The diversity introduced at these interface positions was restricted to amino acids which were predicted to be tolerated based on modeling. Libraries were built, constructed, selected and screened based on methods previously described (Fennell et al, mAbs 2013; 21, 5(6)). Initial screening was carried out in scFv antibody fragment format measuring the ability of mutated scFvs to inhibit the interaction of BDNF with the parental humanized H1 antibody in a HTRF assay using europium-cryptate labeled H1. All clones performing better than the unlabelled parental H1 were reformatted to full-length IgG and screened in the same HTRF assay to quantify fold-improvements over parental. FIG. 10 shows sample data for optimized clones B18, B20 & B30. These improvements were verified using an SPR assay to assess antibody binding to BDNF using a BIAcore T200. Kinetic values are described in Table 12, with SPR curves shown in FIG. 11. Optimized clones were tested to ensure there was no cross-reactivity with other neurotrophins and similarly positively charged chemokines and this data is shown in FIG. 13. The ability of optimized clones to inhibit BDNF-induced signaling was tested in both the TrkB/p75NTR-U2OS Pathfinder assay and the pERK assay and in both cases, optimized clones showed improved activity over the parental humanized H1 clone.

TABLE 12
Calculated Kinetic Constants for Chimeric R3BH1, Humanized H1
and Affinity Optimized Variants B18, B20 & B30.

| | Human BDNF | | | |
|---|---|---|---|---|
| | ka (1/Ms) | kd (1/s) | | |
| Antibody | 1 × E+05 | 1 × E−05 | T_{1/2} (s) | KD (pM) |
| *R3B-H1 | 3.0 ± 0.2 | 1029.3 ± 83.6 | 68 | 34420.0 ± 5431.7 |
| | | | | (n = 3) |
| H1 | 2.9 ± 0.2 | 354.8 ± 56.1 | 200 | 12106.7 ± 1070.0 |
| | | | | (n = 3) |
| *B18 | 2.9 ± 0.2 | 2.8 ± 0.5 | 25308 | 99.4 ± 26.6 |
| | | | | (n = 3) |
| *B20 | 2.8 ± 0.0 | 15.4 ± 0.4 | 4520 | 550.0 ± 23.0 |
| | | | | (n = 2) |
| *B30 | 2.9 ± 0.0 | 3.5 ± 0.3 | 20089 | 120.3 ± 8.9 |
| | | | | (n = 3) |

*Experiments performed at 37° C.

### Example 12

### Identification of Affinity-Optimized H1 Variants Using a H1-BDNF HTRF Assay

A competition homogenous time-resolved fluorescence assay HTRF assay was established to screen for anti-BDNF antibodies that were capable of competing with BDNF bound europium-cryptate labelled humanized H1. Purified H1 was labelled with europium cryptate using a cryptate labeling kit (CisBio) according to manufacturer's instructions. The final assay mixture consisted of 1 nM biotinylated human BDNF, 1/1000 dilution of europium cryptate labeled H1, 1/2000 dilution of SA-XL665 (CisBio), and a dilution series of purified affinity-optimized anti-BDNF antibodies from 0-100 nM in a total reaction volume of 20 μl in 1× assay buffer [50 mM sodium phosphate, pH 7.5, 400 mM potassium fluoride, and 0.1% BSA (w/v)]. Reagents were added sequentially on the MiniTrak Liquid Handling Platform (Perkin-Elmer) into 384-well low volume black plates (Nunc). Reactions were allowed to proceed for 3 hours at room temperature and plates were subsequently read on the EnVision MultiLabel Plate Reader (Perkin-Elmer) with excitation at 340 nm and two emission readings at 615 nm (measuring input donor fluorescence from H1 europium cryptate) and 665 nm (measuring output acceptor fluorescence from SAXL665). All readings were expressed as a ratio of fluorescence at 665/615. The data in FIG. 10 show this 665/615 ratio plotted against the log of antibody concentration (pM) and demonstrates that affinity optimized molecules B18, B20 & B30 specifically bind BDNF and inhibit its interaction with labelled H1 much more effectively than unlabelled H1.

### Example 13

### BDNF Binding Kinetics of Humanized H1 and its Affinity-Optimized Variants B18, B20 & B30

BIAcore experiments to quantitate fold-improvements in affinity for the panel of optimized variants were carried out under conditions described in Example 4 above. Off-rates were considerably slower for optimized clones and for this reason, kinetic determinations were made at 37° C. Calculated kinetic constants from replicate experiments are summarized in Table 12 with representative sensorgrams and extracted off-rate curves shown in FIG. 11.

### Example 14

### Selectivity of Anti-BDNF Antibody B30:Cross-Reactivity with Other Related Neurotrophins & Similarly Positively Charged Chemokines

All affinity optimized clones were screened by titration ELISA on a panel of chemokines and neurotrophins under the conditions described in Example 7 above. FIG. 13 shows only the highest concentration tested for each clone for clarity (300 μL/mL) and indicates that optimized clones have no cross-reactivity with related neurotrophins nor polyspecificity for unrelated highly charged chemokines. FIG. 13 antibody samples are ordered H1, B18, B20, B30, Negative, from front figure row to back figure row.

### Example 15

### Crystal Structure of BDNF-Homodimer in Complex with the Neutralizing Antibody Fragment B30-Fab

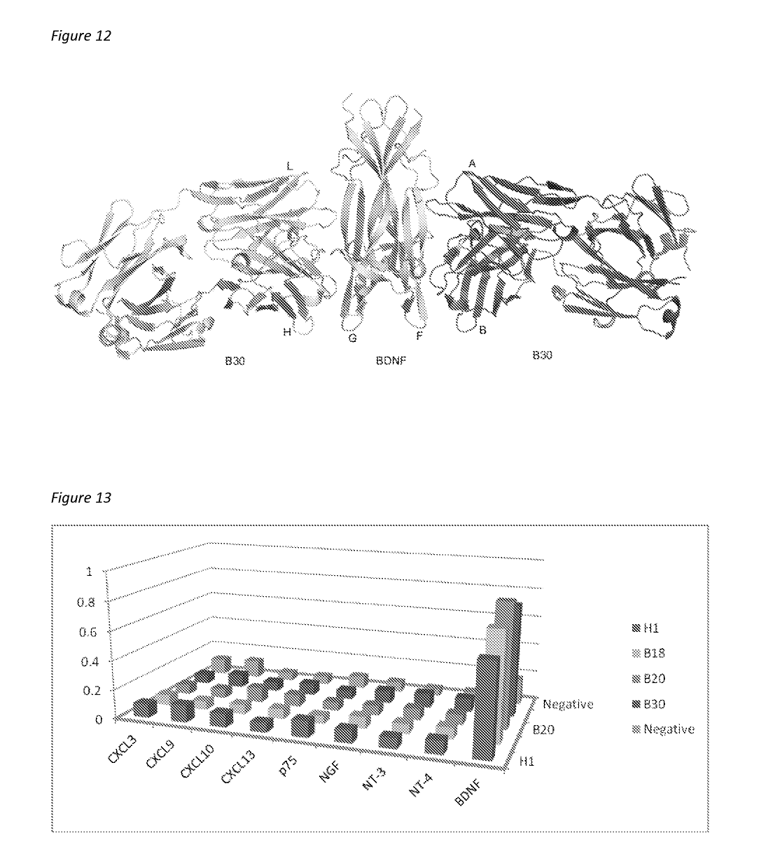

The crystal structure reveals two B30-Fab molecules binding to the two symmetrical opposite sides of a single BDNF-homodimeric cytokine, as demonstrated by the cartoon diagram in FIG. 12. As in the case with TrkB-receptor, the binding of B30 to each of the opposite sides of BDNF creates two interacting surfaces and hence the two binding epitopes on the cytokine surface.

As both these binding surfaces involve similar interactions, details displayed in Table 13a,b refer to only one epitope, that involves the antibody heavy (H) and light (L) chains, and the BDNF-cytokine chains (F and G), chain G is denoted by an asterisk. Table 14 a and b lists all atoms in contact covering both binding surfaces.

The amino acid residues contributing to the antibody binding paratopes and epitope were determined from the B30+BDNF crystal structure and are listed in Table 13a.

TABLE 13a
B30 + BDNF_paratope - epitope contacts at 4 Å distance

| | The B30 Ab contact residues (within the 4 Å distance |
|---|---|
| Paratope | from BDNF) |
| VH | SER 31, ASP 53, TYR 54, ILE 56, GLU 57, THR 58, |
| domain | TYR 59, LYS 65, TYR 101, ILE 103, TRP 105, |
| | ASN 106, HIS 108 |
| VL | GLY 26, TYR 27, TYR 91, TYR 92 |
| domain | |
| Epitope | BDNF contact residues (within the 4 Å distance from B30) |
| Chain F | ILE 16, SER 17, TRP 19, THR 21, ALA 23, GLU 40, |
| | LYS 41, VAL 44, SER 45, GLN 48, LEU 49, LYS 50, |
| | TYR 52 |
| Chain G | MET 31, SER 32, GLY 33, TRY 86, TRP 100, ARG 101, |
| | PHE 102, ARG 104 |

TABLE 13b
specific B30 paratope - BDNF epitope contact residues within 4 Å

| B30 structural motif and | | BDNF-amino acids |
|---|---|---|
| amino acids | | CHAIN F & CHAIN G* |
| CDR-H1 | SER 31 | LYS 41, LYS 50 |
| CDR-H2 | ASP 53 | GLN 48, LEU 49, LYS 50 |
| | Tyr 54 | VAL 44, SER 45, LEU 49 |
| | Ile 56 | LEU 49, TRP 100*, ARG 101* |
| | GLU 57 | TRP 100*, Arg 101*, PHE 102* |
| | THR 58 | SER 32* |
| Frame Work | TYR 59 | MET 31*, SER 32* |
| | LYS 65 | SER 32* |
| CDR-H3 | TYR 101 | THR 21, ALA 23, GLU 40 |
| | | LYS 50 |
| | ILE 103 | Trp 19, THR 21, LYS 50 |
| | | TYR 52 |
| | TRP 105 | MET 31*, TYR 52 |
| | ASN 106 | TRP 19, MET 31* |
| | HIS 108 | TRP 19 |
| CDR-L1 | GLY 26 | ILE 16 |
| | TYR 27 | SER 17 |
| CDR-L3 | TYR 91 | TYR 86*, ARG 104* |
| | TYR 92 | MET 31*, SER 32*, GLY 33*, ARG 104* |

TABLE 14a
B30 Paratope VL and VH contacts, 4 Å contact residues

| Column 1 | Column 2 | Column 3 | Column 4 | Column 5 |
|---|---|---|---|---|
| R3BH1 Paratope | R3BH1 | BDNF Epitope | BDNF | Distance |
| Atoms | Chain | Atoms | Chain | Å |
| 59(TYR)./CE1 | B | 31(MET)./C [C] | F | 3.81 |
| [C] | | | | |
| 92(TYR)./CD1 | A | 31(MET)./O [O] | F | 3.77 |
| [C] | | | | |
| 59(TYR)./CE1 | B | 31(MET)./O [O] | F | 3.39 |
| [C] | | | | |
| 105(TRP)./CH2 | B | 31(MET)./CB [C] | F | 3.77 |
| [C] | | | | |
| 59(TYR)./CD1 | B | 31(MET)./CG | F | 3.71 |
| [C] | | [C] | | |
| 105(TRP)./CZ3 | B | 31(MET)./CG | F | 3.87 |
| [C] | | [C] | | |
| 105(TRP)./CH2 | B | 31(MET)./CG | F | 3.72 |
| [C] | | [C] | | |
| 59(TYR)./CE1 | B | 31(MET)./CG | F | 3.74 |
| [C] | | [C] | | |
| 106(ASN)./ND2 | B | 31(MET)./SD [S] | F | 3.72 |
| [N] | | | | |
| 105(TRP)./CZ2 | B | 31(MET)./CE [C] | F | 3.93 |
| [C] | | | | |
| 105(TRP)./CH2 | B | 31(MET)./CE [C] | F | 3.67 |
| [C] | | | | |
| 59(TYR)./CE1 | B | 32(SER)./CA [C] | F | 3.98 |
| [C] | | | | |
| 92(TYR)./CE1 | A | 32(SER)./C [C] | F | 3.72 |
| [C] | | | | |
| 65(LYS)./NZ [N] | B | 32(SER)./O [O] | F | 3.71 |
| 92(TYR)./CE1 | A | 32(SER)./O [O] | F | 3.70 |
| [C] | | | | |
| 65(LYS)./CE [C] | B | 32(SER)./O [O] | F | 3.53 |
| 58(THR)./O [O] | B | 32(SER)./OG | F | 3.54 |
| | | [O] | | |
| 59(TYR)./CD1 | B | 32(SER)./OG | F | 3.36 |
| [C] | | [O] | | |
| 59(TYR)./CE1 | B | 32(SER)./OG | F | 3.80 |
| [C] | | [O] | | |
| 92(TYR)./CE1 | A | 33(GLY)./N [N] | F | 3.62 |
| [C] | | 33(GLY)./CA [C] | | 3.49 |
| 92(TYR)./CZ [C] | A | 33(GLY)./CA [C] | F | 3.99 |
| 92(TYR)./OH | A | 33(GLY)./CA [C] | F | 3.59 |
| [O] | | | | |
| 91(TYR)./O [O] | A | 86(TYR)./OH | F | 3.60 |
| | | [O] | | |
| 56(ILE)./CD1[C] | B | 97(ARG)./NE | F | 3.73 |
| | | [N] | | 3.90 |
| | | 97(ARG)./CZ [C] | | |
| 55(GLY)./O [O] | B | 97(ARG)./NH1 | F | 3.72 |
| | | [N] | | |
| 56(ILE)./CA [C] | B | 97(ARG)./NH1 | F | 3.91 |
| | | [N] | | |
| 54(TYR)./CE2 | B | 97(ARG)./NH2 | F | 3.69 |
| [C] | | [N] | | |
| 56(ILE)./CD1[C] | B | 99(GLY)./CA [C] | F | 3.76 |
| 56(ILE)./CG2[C] | B | 100(TRP)./O | F | 3.54 |
| | | [O] | | |
| 57(GLU)./OE2 | B | 100(TRP)./O | F | 3.54 |
| [O] | | [O] | | |
| | | 101(ARG)./CA | | 3.08 |
| | | [C] | | |
| | | 101(ARG)./C | | 3.76 |
| | | [C] | | |
| | | 101(ARG)./CB | | 3.58 |
| | | [C] | | |
| | | 101(ARG)./CG | | 3.54 |
| | | [C] | | |
| 56(ILE)./CG1[C] | B | 101(ARG)./NE | F | 3.83 |
| | | [N] | | |
| | | 101(ARG)./CZ | | 3.96 |
| | | [C] | | |
| 56(ILE)./CD1[C] | B | 101(ARG)./CZ | F | 3.73 |
| | | [C] | | |
| 56(ILE)./CG1[C] | B | 101(ARG)./NH2 | F | 3.81 |
| | | [N] | | |
| 56(ILE)./CD1[C] | B | 101(ARG)./NH2 | F | 3.46 |
| | | [N] | | |
| 57(GLU)./OE2 | B | 102(PHE)./N | F | 3.41 |
| [O] | | [N] | | |
| 91(TYR)./CZ [C] | A | 104(ARG)./CD | F | 3.93 |
| | | [C] | | |
| 91(TYR)./OH | A | 104(ARG)./CD | F | 3.51 |
| [O] | | [C] | | |
| | | 104(ARG)./NE | | 3.89 |
| | | [N] | | |
| 91(TYR)./CE2 | A | 104(ARG)./CZ | F | 3.65 |
| [C] | | [C] | | |
| 91(TYR)./CZ [C] | A | 104(ARG)./CZ | F | 3.80 |
| | | [C] | | |
| 91(TYR)./O [O] | A | 104(ARG)./CZ | F | 3.33 |
| | | [C] | | |
| 91(TYR)./CE2 | A | 104(ARG)./NH1 | F | 3.51 |
| [C] | | [N] | | |
| 91(TYR)./CE1 | A | 104(ARG)./NH1 | F | 3.50 |
| [C] | | [N] | | |
| 91(TYR)./CZ [C] | A | 104(ARG)./NH1 | F | 3.44 |
| | | [N] | | |
| 91(TYR)./O [O] | A | 104(ARG)./NH1 | F | 2.85 |
| | | [N] | | |
| 91(TYR)./C [C] | A | 104(ARG)./NH1 | F | 3.84 |
| | | [N] | | |
| 91(TYR)./CG | A | 104(ARG)./NH1 | F | 3.76 |
| [C] | | [N] | | |
| 91(TYR)./CD2 | A | 104(ARG)./NH1 | F | 3.65 |
| [C] | | [N] | | |
| 91(TYR)./CD1 | A | 104(ARG)./NH1 | F | 3.64 |
| [C] | | [N] | | |
| 92(TYR)./CE1 | A | 104(ARG)./NH2 | F | 3.73 |
| [C] | | [N] | | |
| 92(TYR)./CD1 | A | 104(ARG)./NH2 | F | 3.64 |
| [C] | | [N] | | |
| 91(TYR)./O [O] | A | 104(ARG)./NH2 | F | 2.94 |
| | | [N] | | |
| 26(GLY)./O [O] | A | 16(ILE)./CG1[C] | G | 3.83 |
| 27(TYR)./CZ [C] | A | 16(ILE)./CD1[C] | G | 3.86 |
| 27(TYR)./CE2 | A | 16(ILE)./CD1[C] | G | 3.98 |
| [C] | | | | |
| 26(GLY)./O [O] | A | 16(ILE)./CD1[C] | G | 3.78 |
| 26(GLY)./CA [C] | A | 16(ILE)./CD1[C] | G | 3.43 |
| 26(GLY)./C [C] | A | 16(ILE)./CD1[C] | G | 3.65 |
| 27(TYR)./OH | A | 17(SER)./C [C] | G | 3.96 |
| [O] | | | | |
| 27(TYR)./CE1 | A | 17(SER)./O [O] | G | 3.26 |
| [C] | | | | |
| 27(TYR)./CZ [C] | A | 17(SER)./O [O] | G | 3.65 |
| 27(TYR)./OH | A | 17(SER)./O [O] | G | 3.49 |
| [O] | | 17(SER)./CB [C] | | 3.56 |
| 103(ILE)./CB [C] | B | 19(TRP)./CB [C] | G | 3.71 |
| 103(ILE)./CG1 | B | 19(TRP)./CB [C] | G | 3.81 |
| [C] | | | | |
| 103(ILE)./CG2 | B | 19(TRP)./CG [C] | G | 3.85 |
| [C] | | | | |
| 103(ILE)./CB [C] | B | 19(TRP)./CG [C] | G | 3.64 |
| 103(ILE)./CG2 | B | 19(TRP)./CD1 | G | 3.63 |
| [C] | | [C] | | |
| 106(ASN)./OD1 | B | 19(TRP)./CD1 | G | 3.84 |
| [O] | | [C] | | |
| 103(ILE)./CB [C] | B | 19(TRP)./CD1 | G | 3.58 |
| | | [C] | | |
| 103(ILE)./O [O] | B | 19(TRP)./CD1 | G | 3.76 |
| | | [C] | | |
| 108(HIS)./NE2 | B | 19(TRP)./CD1 | G | 3.93 |
| [N] | | [C] | | |
| 103(ILE)./CG2 | B | 19(TRP)./NE1 | G | 3.70 |
| [C] | | [N] | | |
| 106(ASN)./CG | B | 19(TRP)./NE1 | G | 3.54 |
| [C] | | [N] | | |
| 106(ASN)./OD1 | B | 19(TRP)./NE1 | G | 2.88 |
| [O] | | [N] | | |
| 106(ASN)./ND2 | B | 19(TRP)./NE1 | G | 3.59 |
| [N] | | [N] | | |
| 108(HIS)./NE2 | B | 19(TRP)./NE1 | G | 3.95 |
| [N] | | [N] | | |
| 103(ILE)./CG2 | B | 19(TRP)./CE2 | G | 3.96 |
| [C] | | [C] | | |
| 106(ASN)./OD1 | B | 19(TRP)./CE2 | G | 3.83 |
| [O] | | [C] | | |
| 106(ASN)./ND2 | B | 19(TRP)./CZ2 | G | 3.98 |
| [N] | | [C] | | |
| 101(TYR)./CE2 | B | 23(ALA)./CB [C] | G | 3.94 |
| [C] | | | | |
| 101(TYR)./OH | B | 40(GLU)./CG | G | 3.76 |
| [O] | | [C] | | |
| | | 40(GLU)./CD [C] | | 3.65 |
| 101(TYR)./CZ | B | 40(GLU)./OE2 | G | 3.76 |
| [C] | | [O] | | |
| 101(TYR)./OH | B | 40(GLU)./OE2 | G | 2.78 |
| [O] | | [O] | | |
| 31(SER)./OG | B | 41(LYS)./NZ [N] | G | 3.57 |
| [O] | | | | |
| 54(TYR)./CE1 | B | 44(VAL)./CG2 | G | 3.53 |
| [C] | | [C] | | |
| 54(TYR)./CZ [C] | B | 44(VAL)./CG2 | G | 3.63 |
| | | [C] | | |
| 54(TYR)./OH | B | 44(VAL)./CG2 | G | 3.84 |
| [O] | | [C] | | |
| | | 45(SER)./C [C] | | 3.80 |
| 54(TYR)./CE1 | B | 45(SER)./O [O] | G | 3.90 |
| [C] | | | | |
| 54(TYR)./CZ [C] | B | 45(SER)./O [O] | G | 3.65 |
| 54(TYR)./OH | B | 45(SER)./O [O] | G | 2.60 |
| [O] | | 45(SER)./OG | | 3.81 |
| | | [O] | | |
| 53(ASP)./OD2 | B | 48(GLN)./O [O] | G | 3.90 |
| [O] | | | | |
| 54(TYR)./CG | B | 49(LEU)./CD1 | G | 3.76 |
| [C] | | [C] | | |
| 54(TYR)./CB [C] | B | 49(LEU)./CD1 | G | 3.43 |
| | | [C] | | |
| 54(TYR)./CD2 | B | 49(LEU)./CD1 | G | 3.90 |
| [C] | | [C] | | |
| 53(ASP)./OD2 | B | 50(LYS)./N [N] | G | 3.42 |
| [O] | | 50(LYS)./CB [C] | | 3.76 |
| 53(ASP)./CG | B | 50(LYS)./CB [C] | G | 3.86 |
| [C] | | | | |
| 53(ASP)./OD1 | B | 50(LYS)./CB [C] | G | 3.67 |
| [O] | | | | |
| 101(TYR)./OH | B | 50(LYS)./CG [C] | G | 3.54 |
| [O] | | | | |
| 31(SER)./O [O] | B | 50(LYS)./CD [C] | G | 3.58 |
| 101(TYR)./CZ | B | 50(LYS)./CD [C] | G | 3.91 |
| [C] | | | | |
| 101(TYR)./OH | B | 50(LYS)./CD [C] | G | 3.90 |
| [O] | | | | |
| 31(SER)./O [O] | B | 50(LYS)./CE [C] | G | 3.65 |
| 101(TYR)./CE1 | B | 50(LYS)./CE [C] | G | 3.74 |
| [C] | | | | |
| 101(TYR)./CE2 | B | 50(LYS)./CE [C] | G | 3.78 |
| [C] | | | | |
| 101(TYR)./CZ | B | 50(LYS)./CE [C] | G | 3.59 |
| [C] | | | | |
| 103(ILE)./CD1 | B | 50(LYS)./CE [C] | G | 3.76 |
| [C] | | | | |
| 101(TYR)./O | B | 50(LYS)./CE [C] | G | 3.34 |
| [O] | | | | |
| 31(SER)./C [C] | B | 50(LYS)./NZ [N] | G | 3.90 |
| 31(SER)./O [O] | B | 50(LYS)./NZ [N] | G | 2.78 |
| 101(TYR)./O | B | 50(LYS)./NZ [N] | G | 2.87 |
| [O] | | | | |
| 101(TYR)./C | B | 50(LYS)./NZ [N] | G | 3.85 |
| [C] | | | | |
| 103(ILE)./CG1 | B | 52(TYR)./CG [C] | G | 3.70 |
| [C] | | | | |
| 103(ILE)./CD1 | B | 52(TYR)./CG [C] | G | 3.72 |
| [C] | | 52(TYR)./CD1 | | 3.49 |
| | | [C] | | |
| 103(ILE)./CG1 | B | 52(TYR)./CD2 | G | 3.65 |
| [C] | | [C] | | |
| 103(ILE)./CD1 | B | 52(TYR)./CE1 | G | 3.71 |
| [C] | | [C] | | |
| 103(ILE)./CG2 | B | 52(TYR)./CE2 | G | 3.63 |
| [C] | | [C] | | |
| 103(ILE)./CG1 | B | 52(TYR)./CE2 | G | 3.97 |
| [C] | | [C] | | |
| 103(ILE)./CG2 | B | 52(TYR)./CZ [C] | G | 3.82 |
| [C] | | | | |
| 105(TRP)./NE1 | B | 52(TYR)./CZ [C] | G | 3.96 |
| [N] | | | | |
| 103(ILE)./CG2 | B | 52(TYR)./OH | G | 3.98 |
| [C] | | [O] | | |
| 105(TRP)./CD1 | B | 52(TYR)./OH | G | 3.70 |
| [C] | | [O] | | |
| 105(TRP)./NE1 | B | 52(TYR)./OH | G | 2.92 |
| [N] | | [O] | | |
| 105(TRP)./CE2 | B | 52(TYR)./OH | G | 3.92 |
| [C] | | [O] | | |

TABLE 14b
B30 Paratope VL and VH contacts, 4 Å contact residues

| | Column 2 | | | |
|---|---|---|---|---|
| Column 1 | R3BH1 | Column 3 | Column 4 | Column 5 |
| R3BH1 Paratope | variable | BDNF Epitope | BDNF | Distance |
| Atoms | region | Atoms | Chain | Å |
| 26(GLY)./C [C] | L | 16(ILE)./CD1[C] | F | 3.91 |
| 26(GLY)./O [O] | L | 16(ILE)./CD1[C] | F | 3.20 |
| 27(TYR)./CE1 | L | 17(SER)./O [O] | F | 3.52 |
| [C] | | | | |
| 27(TYR)./CZ [C] | L | 17(SER)./O [O] | F | 3.91 |
| 27(TYR)./OH | L | 17(SER)./O [O] | F | 3.77 |
| [O] | | 17(SER)./CB [C] | | 3.62 |
| 103(ILE)./CB [C] | H | 19(TRP)./CB [C] | F | 3.71 |
| 103(ILE)./CG1 | H | 19(TRP)./CB [C] | F | 3.82 |
| [C] | | | | |
| 103(ILE)./CB [C] | H | 19(TRP)./CG [C] | F | 3.66 |
| 103(ILE)./CG2 | H | 19(TRP)./CG [C] | F | 3.89 |
| [C] | | | | |
| 103(ILE)./O [O] | H | 19(TRP)./CD1 | F | 3.74 |
| | | [C] | | |
| 103(ILE)./CB [C] | H | 19(TRP)./CD1 | F | 3.65 |
| | | [C] | | |
| 103(ILE)./CG2 | H | 19(TRP)./CD1 | F | 3.74 |
| [C] | | [C] | | |
| 106(ASN)./OD1 | H | 19(TRP)./CD1 | F | 3.82 |
| [O] | | [C] | | |
| 108(HIS)./NE2 | H | 19(TRP)./CD1 | F | 3.84 |
| [N] | | [C] | | |
| 103(ILE)./CG2 | H | 19(TRP)./NE1 | F | 3.82 |
| [C] | | [N] | | |
| 106(ASN)./CG | H | 19(TRP)./NE1 | F | 3.50 |
| [C] | | [N] | | |
| 106(ASN)./OD1 | H | 19(TRP)./NE1 | F | 2.83 |
| [O] | | [N] | | |
| 106(ASN)./ND2 | H | 19(TRP)./NE1 | F | 3.53 |
| [N] | | [N] | | |
| 108(HIS)./CD2 | H | 19(TRP)./NE1 | F | 3.99 |
| [C] | | [N] | | |
| 108(HIS)./NE2 | H | 19(TRP)./NE1 | F | 3.83 |
| [N] | | [N] | | |
| 103(ILE)./CG2 | H | 19(TRP)./CE2 | F | 3.96 |
| [C] | | [C] | | |
| 106(ASN)./OD1 | H | 19(TRP)./CE2 | F | 3.72 |
| [O] | | [C] | | 3.99 |
| | | 19(TRP)./CZ2 | | |
| | | [C] | | |
| 106(ASN)./ND2 | H | 19(TRP)./CZ2 | F | 3.88 |
| [N] | | [C] | | |
| 101(TYR)./CE2 | H | 21(THR)./CB [C] | F | 3.95 |
| [C] | | 21(THR)./CG2 | | 3.93 |
| | | [C] | | |
| 103(ILE)./CD1 | H | 21(THR)./CG2 | F | 3.82 |
| [C] | | [C] | | |
| 101(TYR)./OH | H | 23(ALA)./CB [C] | F | 3.86 |
| [O] | | | | |
| 101(TYR)./CE2 | H | 23(ALA)./CB [C] | F | 3.74 |
| [C] | | | | |
| 101(TYR)./OH | H | 40(GLU)./CG | F | 3.39 |
| [O] | | [C] | | 3.72 |
| | | 40(GLU)./CD [C] | | |
| 31(SER)./CB | H | 41(LYS)./NZ [N] | F | 3.90 |
| [C] | | | | |
| 31(SER)./OG | H | 41(LYS)./NZ [N] | F | 3.11 |
| [O] | | | | |
| 54(TYR)./CE1 | H | 44(VAL)./CG2 | F | 3.37 |
| [C] | | [C] | | |
| 54(TYR)./CZ | H | 44(VAL)./CG2 | F | 3.52 |
| [C] | | [C] | | |
| 54(TYR)./OH | H | 44(VAL)./CG2 | F | 3.77 |
| [O] | | [C] | | |
| 54(TYR)./CD1 | H | 44(VAL)./CG2 | F | 3.88 |
| [C] | | [C] | | |
| 54(TYR)./OH | H | 45(SER)./C [C] | F | 3.72 |
| [O] | | | | |
| 54(TYR)./CE1 | H | 45(SER)./O [O] | F | 3.78 |
| [C] | | | | |
| 54(TYR)./CZ | H | 45(SER)./O [O] | F | 3.56 |
| [C] | | | | |
| 54(TYR)./OH | H | 45(SER)./O [O] | F | 2.53 |
| [O] | | 45(SER)./OG | | 3.73 |
| | | [O] | | |
| 53(ASP)./OD2 | H | 48(GLN)./O [O] | F | 3.73 |
| [O] | | 49(LEU)./CA [C] | | 3.92 |
| 54(TYR)./CB | H | 49(LEU)./CD1 | F | 3.81 |
| [C] | | [C] | | |
| 56(ILE)./CD1 | H | 49(LEU)./CD1 | F | 3.89 |
| [C] | | [C] | | 3.94 |
| | | 49(LEU)./CD2 | | |
| | | [C] | | |
| 53(ASP)./OD2 | H | 50(LYS)./N [N] | F | 3.21 |
| [O] | | | | |
| 53(ASP)./CG | H | 50(LYS)./N [N] | F | 3.91 |
| [C] | | | | |
| 53(ASP)./OD1 | H | 50(LYS)./N [N] | F | 3.90 |
| [O] | | | | |
| 53(ASP)./OD2 | H | 50(LYS)./CB [C] | F | 3.75 |
| [O] | | | | |
| 53(ASP)./CG | H | 50(LYS)./CB [C] | F | 3.78 |
| [C] | | | | |
| 53(ASP)./OD1 | H | 50(LYS)./CB [C] | F | 3.55 |
| [O] | | | | |
| 53(ASP)./OD2 | H | 50(LYS)./CG [C] | F | 3.77 |
| [O] | | | | |
| 101(TYR)./OH | H | 50(LYS)./CG [C] | F | 3.97 |
| [O] | | | | |
| 53(ASP)./CG | H | 50(LYS)./CG [C] | F | 3.84 |
| [C] | | | | |
| 31(SER)./O [O] | H | 50(LYS)./CD [C] | F | 3.32 |
| 53(ASP)./OD2 | H | 50(LYS)./CD [C] | F | 3.91 |
| [O] | | | | |
| 53(ASP)./CB | H | 50(LYS)./CD [C] | F | 3.75 |
| [C] | | | | |
| 53(ASP)./CG | H | 50(LYS)./CD [C] | F | 3.54 |
| [C] | | | | |
| 53(ASP)./OD1 | H | 50(LYS)./CD [C] | F | 3.68 |
| [O] | | | | |
| 101(TYR)./CE1 | H | 50(LYS)./CE [C] | F | 3.81 |
| [C] | | | | |
| 31(SER)./O [O] | H | 50(LYS)./CE [C] | F | 3.53 |
| 101(TYR)./CZ | H | 50(LYS)./CE [C] | F | 3.71 |
| [C] | | | | |
| 101(TYR)./CE2 | H | 50(LYS)./CE [C] | F | 3.96 |
| [C] | | | | |
| 101(TYR)./O | H | 50(LYS)./CE [C] | F | 3.46 |
| [O] | | | | |
| 103(ILE)./CD1 | H | 50(LYS)./CE [C] | F | 3.76 |
| [C] | | | | |
| 31(SER)./O [O] | H | 50(LYS)./NZ [N] | F | 3.24 |
| 101(TYR)./O | H | 50(LYS)./NZ [N] | F | 2.79 |
| [O] | | | | |
| 101(TYR)./C | H | 50(LYS)./NZ [N] | F | 3.85 |
| [C] | | | | |
| 103(ILE)./CD1 | H | 50(LYS)./NZ [N] | F | 3.54 |
| [C] | | | | |
| 103(ILE)./CG1 | H | 52(TYR)./CG [C] | F | 3.68 |
| [C] | | | | |
| 103(ILE)./CD1 | H | 52(TYR)./CG [C] | F | 3.68 |
| [C] | | 52(TYR)./CD1 | | 3.53 |
| | | [C] | | |
| 103(ILE)./CG1 | H | 52(TYR)./CD2 | F | 3.61 |
| [C] | | [C] | | |
| 103(ILE)./CD1 | H | 52(TYR)./CE1 | F | 3.82 |
| [C] | | [C] | | |
| 103(ILE)./CG1 | H | 52(TYR)./CE2 | F | 3.97 |
| [C] | | [C] | | |
| 103(ILE)./CG2 | H | 52(TYR)./CE2 | F | 3.50 |
| [C] | | [C] | | |
| 105(TRP)./NE1 | H | 52(TYR)./CZ [C] | F | 3.81 |
| [N] | | | | |
| 103(ILE)./CG2 | H | 52(TYR)./CZ [C] | F | 3.76 |
| [C] | | | | |
| 105(TRP)./NE1 | H | 52(TYR)./OH | F | 2.80 |
| [N] | | [O] | | |
| 105(TRP)./CE2 | H | 52(TYR)./OH | F | 3.82 |
| [C] | | [O] | | |
| 103(ILE)./CG2 | H | 52(TYR)./OH | F | 4.00 |
| [C] | | [O] | | |
| 105(TRP)./CD1 | H | 52(TYR)./OH | F | 3.59 |
| [C] | | [O] | | |
| 59(TYR)./CE1 | H | 31(MET)./C [C] | G | 3.97 |
| [C] | | 31(MET)./O [O] | | 3.56 |
| 92(TYR)./CD1 | L | 31(MET)./O [O] | G | 3.57 |
| [C] | | | | |
| 92(TYR)./CE1 | L | 31(MET)./O [O] | G | 3.76 |
| [C] | | | | |
| 105(TRP)./CH2 | H | 31(MET)./CB [C] | G | 3.89 |
| [C] | | 31(MET)./CG | | 3.84 |
| | | [C] | | |
| 59(TYR)./CD1 | H | 31(MET)./CG | G | 3.72 |
| [C] | | [C] | | |
| 59(TYR)./CE1 | H | 31(MET)./CG | G | 3.69 |
| [C] | | [C] | | |
| 106(ASN)./ND2 | H | 31(MET)./SD [S] | G | 3.97 |
| [N] | | | | |
| 105(TRP)./CH2 | H | 31(MET)./CE [C] | G | 3.76 |
| [C] | | | | |
| 92(TYR)./CE1 | L | 32(SER)./C [C] | G | 3.56 |
| [C] | | | | |
| 65(LYS)./NZ | H | 32(SER)./C [C] | G | 3.98 |
| [N] | | | | |
| 92(TYR)./CE1 | L | 32(SER)./O [O] | G | 3.55 |
| [C] | | | | |
| 65(LYS)./CE | H | 32(SER)./O [O] | G | 3.57 |
| [C] | | | | |
| 65(LYS)./NZ | H | 32(SER)./O [O] | G | 3.00 |
| [N] | | | | |
| 92(TYR)./OH | L | 32(SER)./O [O] | G | 3.96 |
| [O] | | | | |
| 58(TH R)./O [O] | H | 32(SER)./OG | G | 3.56 |
| | | [O] | | |
| 59(TYR)./CD1 | H | 32(SER)./OG | G | 3.53 |
| [C] | | [O] | | |
| 59(TYR)./CE1 | H | 32(SER)./OG | G | 3.98 |
| [C] | | [O] | | |
| 92(TYR)./CE1 | L | 33(GLY)./N [N] | G | 3.62 |
| [C] | | 33(GLY)./CA [C] | | 3.72 |
| 92(TYR)./CZ [C] | L | 33(GLY)./CA [C] | G | 4.00 |
| 92(TYR)./OH | L | 33(GLY)./CA [C] | G | 3.33 |
| [O] | | | | |
| 91(TYR)./O [O] | L | 86(TYR)./OH | G | 3.63 |
| | | [O] | | |
| 56(ILE)./CG2 | H | 100(TRP)./O | G | 3.78 |
| [C] | | [O] | | |
| 56(ILE)./CD1 | H | 100(TRP)./O | G | 3.30 |
| [C] | | [O] | | |
| 57(GLU)./OE2 | H | 100(TRP)./O | G | 3.55 |
| [O] | | [O] | | 3.08 |
| | | 101(ARG)./CA | | 3.79 |
| | | [C] | | 3.51 |
| | | 101(ARG)./C | | |
| | | [C] | | |
| | | 101(ARG)./CB | | |
| | | [C] | | |
| 56(ILE)./CG2 | H | 101(ARG)./CG | G | 3.92 |
| [C] | | [C] | | |
| 57(GLU)./OE2 | H | 101(ARG)./CG | G | 3.43 |
| [O] | | [C] | | |
| 56(ILE)./CG2 | H | 101(ARG)./NE | G | 3.81 |
| [C] | | [N] | | |
| 57(GLU)./OE2 | H | 102(PHE)./N | G | 3.45 |
| [O] | | [N] | | |
| 91(TYR)./OH | L | 104(ARG)./CD | G | 3.59 |
| [O] | | [C] | | 3.77 |
| | | 104(ARG)./NE | | |
| | | [N] | | |
| 91(TYR)./O [O] | L | 104(ARG)./CZ | G | 3.63 |
| | | [C] | | |
| 91(TYR)./CE2 | L | 104(ARG)./CZ | G | 3.60 |
| [C] | | [C] | | |
| 91(TYR)./CZ [C] | L | 104(ARG)./CZ | G | 3.65 |
| | | [C] | | |
| 91(TYR)./OH | L | 104(ARG)./CZ | G | 3.89 |
| [O] | | [C] | | |
| 91(TYR)./O [O] | L | 104(ARG)./NH1 | G | 3.20 |
| | | [N] | | |
| 91(TYR)./CD2 | L | 104(ARG)./NH1 | G | 3.77 |
| [C] | | [N] | | |
| 91(TYR)./CE2 | L | 104(ARG)./NH1 | G | 3.49 |
| [C] | | [N] | | |
| 91(TYR)./CE1 | L | 104(ARG)./NH1 | G | 3.38 |
| [C] | | [N] | | |
| 91(TYR)./CZ [C] | L | 104(ARG)./NH1 | G | 3.31 |
| | | [N] | | |
| 91(TYR)./OH | L | 104(ARG)./NH1 | G | 3.82 |
| [O] | | [N] | | |
| 91(TYR)./CG | L | 104(ARG)./NH1 | G | 3.87 |
| [C] | | [N] | | |
| 91(TYR)./CD1 | L | 104(ARG)./NH1 | G | 3.65 |
| [C] | | [N] | | |
| 92(TYR)./CD1 | L | 104(ARG)./NH2 | G | 3.77 |
| [C] | | [N] | | |
| 92(TYR)./CE1 | L | 104(ARG)./NH2 | G | 3.65 |
| [C] | | [N] | | |
| 91(TYR)./O [O] | L | 104(ARG)./NH2 | G | 3.15 |
| | | [N] | | |
| 91(TYR)./CE2 | L | 104(ARG)./NH2 | G | 3.81 |
| [C] | | [N] | | |

### Example 16

### Humanised Anti-BDNF, B30 Inhibits the Activity of BDNF at the TrkB and p75NTR Receptors in TrkB/p75NTR Expressing Cells

In a similar experimental conditions to Example 8 above, R3BH1 and the affinity-optimised variants B18, B20 and B30 were run in the pERK (phospho-extracellular signal-regulated kinase) assay to demonstrate their effects on the functional activity of BDNF at TrkB receptors. U2OS cells expressing TrkB+p75NTR (DiscoverX Corp.) were plated into a 96 well plate in minimum essential medium (MEM; Life Technologies)+0.5% horse serum (Life Technologies) at 100,000 cells per well, 100 ul volume, and left in a 37° C. incubator overnight.

On the day of the assay, R3b-H1, B18, B20, B30 and TrkB-Fc were serial diluted 1:3 in phosphate buffered solution to create a 10 point concentration response curve. 10 ul of the serial diluted samples were then added to the cells and incubated for 1 h at 37° C., following the 1 h incubation, 10 ul of 1.8 nM BDNF (Peprotech) in PBS+0.25% BSA was added to each well, BDNF final assay concentration (FAC): 150 pM. The plate was incubated for 30 minutes at room temperature before media removal and the addition of 35 ul Cellul'erk lysis buffer (Cisbio). The plate was then stored at −80° C. overnight. After thawing, 16 ul of the lysates were transferred to a 384 well Proxiplate (Perkin Elmer) and 8 ul Cellul'erk HTRF reagents added as per kit instructions. After incubating at room temperature for 2 h, the plate was read using a HTRF protocol on an Envision plate reader (Perkin Elmer). Concentration response curves were observed following analysis in Graphpad Prism.

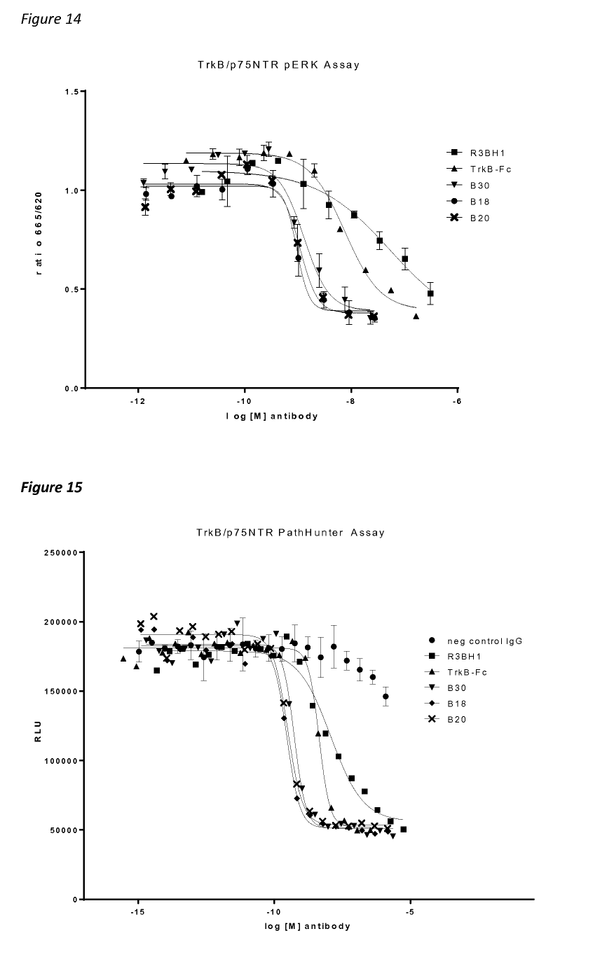

As shown in FIG. 14, presence of the anti-BDNF antibodies inhibited the BDNF mediated activation of the TrkB receptor and subsequent activation of pERK in the cells. IC5Os for TrkBFc, R3BH1, B18, B20 and B30 were 7.6 nM, 53.6 nM, 0.95 nM, 1.1 nM and 1.3 nM respectively, thereby functionally demonstrating improved ligand neutralising properties of the affinity-optimised variants over R3BH1 and also over TrkBFc.

### Example 17

### Humanised Anti-BDNF Molecule, B30 Inhibits the Activity of BDNF at TrkB Receptors in Recombinant TrkB/p75NTR Cells Using the PathHunter pTrkB Assay

In similar experimental conditions to Example 9 above, R3BH1 and the affinity-optimised variants B18, B20 and B30 were run in the DiscoverX PathHunter assay to demonstrate their BDNF neutralising activity at TrkB receptors. U2OS cells expressing TrkB+p75NTR (DiscoverX Corp.) were plated into a 384 well TC plate in minimum essential medium (MEM; Life Technologies)+0.5% horse serum (Life Technologies) at 10,000 cells per well, 40 ul volume, and left in a 37 deg Celsius incubator overnight.

On the day of the assay, R3b-H1, B18, B20, B30, TrkB-Fc and an IgG isotype control were serialised 1:3 in phosphate buffered solution to create 20 point concentration response curves. 10 ul of the serialised samples were then added to the cells and incubated for 1 h at 37 deg Celsius. Following the 1 h incubation, 10 ul of 1.8 nM BDNF (Peprotech) in PBS+0.25% BSA was added to each well, BDNF final assay concentration (FAC): 300 pM. The plate was incubated for 3 h at room temperature before the addition of 20 ul per well of PathHunter Detection reagent (DiscoverX Corp.) and the plate left at room temperature for 1 h before reading luminesence on an Envision plate reader (Perkin Elmer). Concentration response curves were observed following analysis in Graphpad Prism.

As shown in FIG. 15, presence of the anti-BDNF antibodies inhibited the BDNF mediated activation of the TrkB receptor in the Pathhunter assay. IC50s for TrkBFc, R3BH1, B18, B20 and B30 were 4.4 nM, 11.7 nM, 0.29 nM, 0.31 nM and 0.54 nM respectively, thereby functionally demonstrating improved ligand neutralising properties of the affinity-optimised variants over R3BH1 and also over TrkBFc.

### Example 18

### Anti-BDNF R3b-H1 Antibody and B30 Specifically Binds BDNF as a Stable Complex in a Dose Dependant Manner from an In-Vivo Obtained Biological Fluid

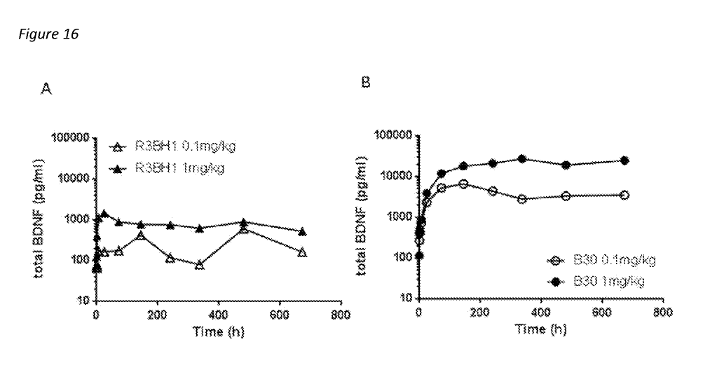

Total BDNF (free and antibody bound) was quantified in rat plasma using a ligand-binding assay following an intravenous dose of anti-BDNF R3b-H1 MAb at 0.1 and 1 mg/kg. In the assay, a commercially available mouse anti-human BDNF biotinylated monoclonal antibody was captured onto streptavidin beads on the affinity capture column [Gyrolab CD microstructure]. BDNF standards, controls and plasma samples from the in vivo study were pre-incubated with excess anti-BDNF R3b-H1 or B30 antibody, to complex available BDNF target. The BDNF/anti-BDNF MAb complex is captured onto the affinity capture column via the mouse anti-human BDNF biotin MAb and the complex was detected with Alexa 647 labeled donkey anti-human IgG (H+L). The fluorescent signal on the column allows for detection of the bound BDNF/anti-BDNF complex. Sample concentrations were determined by interpolation from a standard curve that was fitted using a 5-parameter logistic curve fit with 1/y² response weighting. Data for the anti-BDNF R3b-H1 and B30 antibody dosed rats and total BDNF levels (free+bound) in plasma samples over the study period is shown in FIGS. 16A and 16B, respectively. The data demonstrates that anti-BDNF R3b-H1 and B30 antibody specifically bind endogenous BDNF in a stable complex in plasma in vivo. Dosing of animals with the humanized affinity matured anti-BDNF molecule, B30 resulted in significantly greater BDNF binding in vivo, as shown in FIG. 16B. BDNF levels increased in a dose dependent manner, remaining elevated at 672 hours. The antibody therefore shows selective binding for BDNF and leads to the formation of a stable complex with a longer half life than unbound BDNF.

### Example 19

### In Vivo Efficacy in a Nerve Injury Model; Effect of the Anti-BDNF Antibody R3BH1 and Humanised Affinity Matured Anti-BDNF Molecule B30 Upon Injury Induced Ion Channel Plasticity in Rat Dorsal Root Ganglion (DRG) Neurons

Injury to peripheral nerves often results in neuropathic pain. The mechanisms underlying this condition are complex and involve changes occurring at different levels of the nervous system, one of which involves changes in the expression pattern of ion channels leading to altered neuronal excitability. Contributing to this neuropathy is dysregulation of voltage gated potassium (Kᵥ) channels. Kv channels are key regulators of neuronal excitability and govern the frequency of action potential firing. One hallmark feature of peripheral nerve injury is reduction in the current conducted by Kv ion channels and an increased neuronal excitability. Similar observations have been reported in models of inflammatory pain.

Reduction of Kv ion channels is intrinsically linked to increased excitability and represents a surrogate measure of pain hypersensitivity in neuropathic animals. Kᵥ downregulation has been reported to be mediated by elevated BDNF expression following injury (Cao et al J Neurochem, 114, p1460, 2010). Here we demonstrate that systemic administration of anti-BDNF antibody R3BH1 reverses injury induced Kv suppression in a dose dependent manner. Additionally, humanised anti-BDNF antibody B30 reverses alterations in Kv current induced by nerve injury in this rat model of neuropathic pain. The current carried by Kᵥ ion channels was measured electrophysiologically in injured and uninjured DRG neurons.

DRG at spinal levels lumbar 5 and 6 were dissected from rats either ipsilateral or contralateral to the spinal nerve ligation (SNL) surgical procedure; L5 and L6 DRG from the same side were pooled and dissociated. DRGs were digested in medium containing collagenase then incubated in medium containing trypsin. Following washing and trituration, the dissociated cells were centrifuged, resuspended and plated on glass coverslips. All subsequent recordings were made on the same day as the dissociation. Voltage-clamp recordings were performed from a V_{hold} of −90 mV and then stepped to +60 mV in 10 mV increments. The delayed-rectifier currents (Iₖ quantified at the end of the test pulse) were quantified in subsequent analyses. All measured currents were normalised to the cell's size as measured by cellular capacitance resulting in current densities (pA/pF).

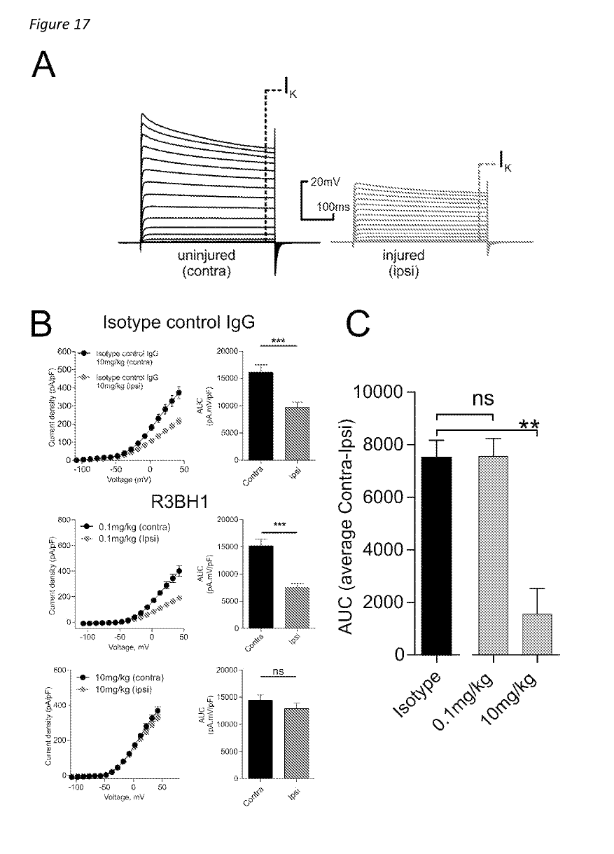

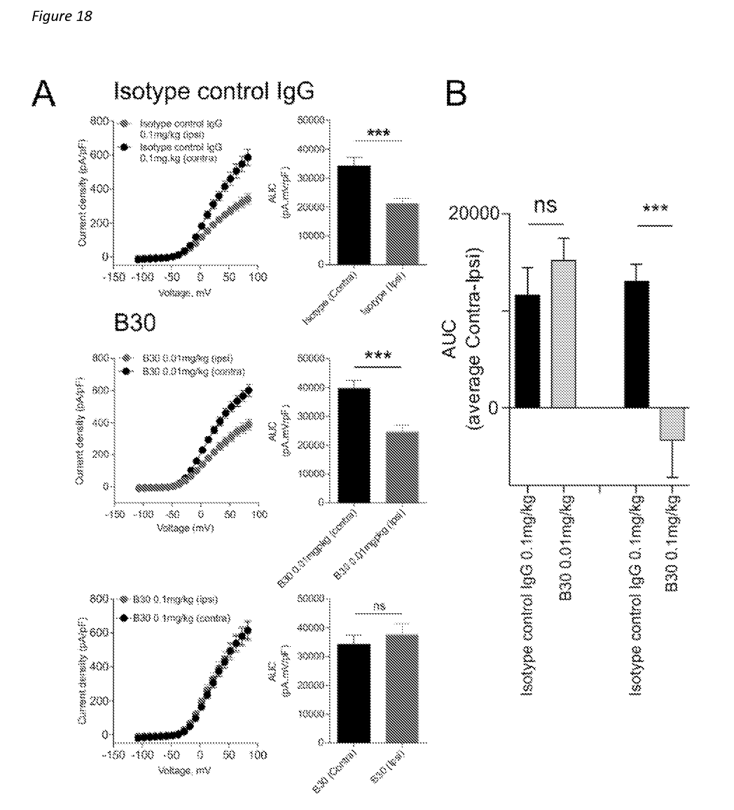

At 3 weeks post injury, DRG neurons recorded from the ipsilateral side to the injury exhibited a smaller current size when compared to uninjured contralateral neurons in the same animal (FIG. 17A). Animals treated with the isotype control IgG (negative control) displayed a clear injury response (FIGS. 17B top panel and 17C) while rats dosed with the anti-BDNF molecule, R3BH1, exhibited a dose dependent reversal of I_{K} suppression back to non-injured levels. Full reversal was obtained with R3BH1 10 mg/kg dose, but not the R3BH1 0.1 mg/kg dose (FIGS. 17B middle and lower panel and 17C). The humanised clone, B30, given at a dose of 0.1 mg/kg fully reversed the injury phenotype, confirming improved potency of the affinity-matured molecule (FIGS. 18A and 18B). Taken together, these data demonstrate the potential utility of R3BH1 and B30 in interfering with mechanisms that drive chronic pain and hence in treating pain, such as chronic pain, neuropathic pain and symptoms, conditions and diseases associated with such pain.

### Example 20

### Humanised Affinity Matured Anti-BDNF Molecule (B30) Reduces Primary Afferent Fibre Hyperexcitability in a Skin Nerve Recording Assay

Nerve injury is known to cause mechanical and heat sensitisation of primary afferent fibres which typically results in reduced activation thresholds and enhanced firing response to evoked stimuli.

The activity of B30 on primary afferent hyperexcitability was evaluated 3 weeks after spinal nerve ligation using the skin nerve preparation. Animals were dosed with either humanised anti-BDNF antibody B30 or inactive isotype (hIgG1) 3-5 days before the day of the experiment. The tibial nerve, along with the associated glaborous skin, was dissected free as described previously (Zimmerman K, et al. Nat Protoc 2009; 4(2); 174-96). The skin is placed, glaborous side down, in a chamber that is continually superfused with oxygenated (95% 02, 5% CO2) modified Krebs' solution maintained at 36±1° C. A section of desheathed nerve fibre was placed in a suction electrode for afferent nerve recording and the electrical activity was recorded.

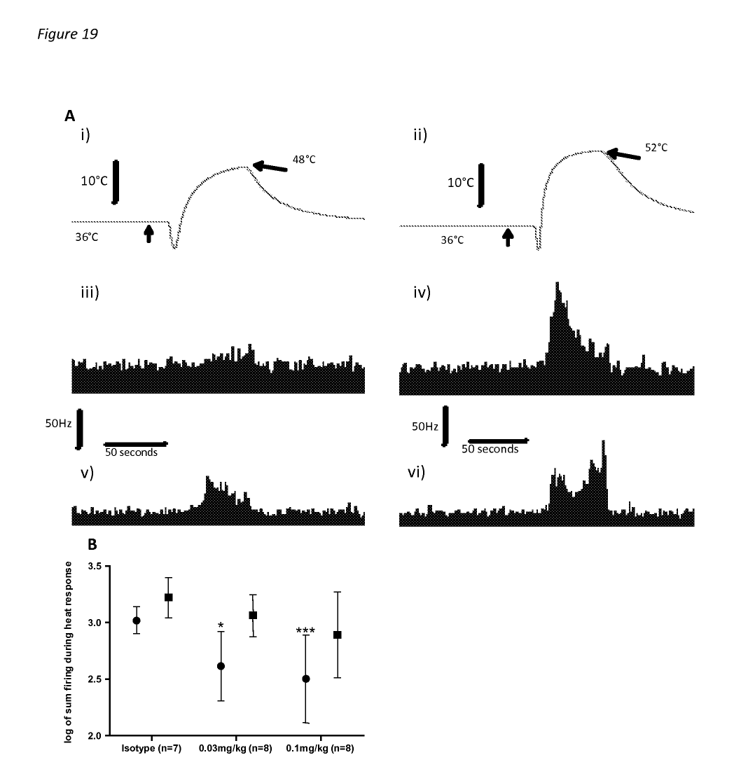

A heat stimulus, consisting of hot Krebs flowed onto the skin over 50 seconds was applied to each preparation. The heat was delivered either via a slow ramp where the rate of temperature rise was slow (36±1° C. to 48±1° C., see FIG. 19Ai), or via a fast ramp where temperature rose more rapidly to 52±1° C. in 50 seconds (see FIG. 19Aii). The two ramps were delivered 15 minutes apart. Slow ramp heat stimulation normally elicits a low firing frequency response in the absence of injury (Aiii), however, following nerve injury, the same stimulus evokes a high frequency firing in the injured leg (Av). This is indicative of heat sensitisation in peripheral nerve fibres. At the end of the experiment, 1 mM lidocaine was superfused onto the preparation for 15 minutes to remove all physiological activity.

The humanised BDNF antibody B30 significantly reversed thermal hypersensitivity in the skin-nerve preparation as shown by the dose dependent reduction in nerve firing in response to slow heat ramp stimulation. The data suggests that B30 has potential utility in reversing mechanisms underlying peripheral nerve hyperexcitability following peripheral nerve damage.

### Example 21

### Humanised Affinity Matured Anti-BDNF Molecule B30 Reduces Spinal Dorsal Horn Neuronal Excitability In Vivo

Peripheral nerve injury results in neuronal excitability changes at multiple levels of the pain neuraxis. Enhanced primary afferent input generates a state of central sensitisation in the spinal cord, that can amplify pain signalling and contribute to long lasting alterations in pain sensory processing. Following tibial nerve transection, there is evidence for spinal cord sensitisation and this is manifested as exaggerated responses to evoked inputs such as mechanical punctate stimulation and cold. To investigate whether BDNF plays a role in mediating hyperexcitability of spinal neurones, animals were dosed with the humanised anti-BDNF antibody and the response profile of spinal dorsal horn neurones was characterised to a range of modalities.

Two to three weeks following tibial nerve injury, animals were systemically dosed with the anti-BDNF molecule B30 and in vivo electrophysiology was conducted 4-7 days later. Animals were anesthetised with isoflurane and the body temperature was monitored and maintained at 37° C. through the use of a heating blanket. A laminectomy was performed to expose the region of the spinal cord receiving afferent input from the hindpaw of the rat. On identification of a single unit, the ongoing activity of the neurone was quantified prior to stimulus application. A range of natural stimuli was delivered to the centre of the receptive field. This included application of mechanical punctate stimuli, delivered through von Frey filaments, and heat, delivered via a water jet.

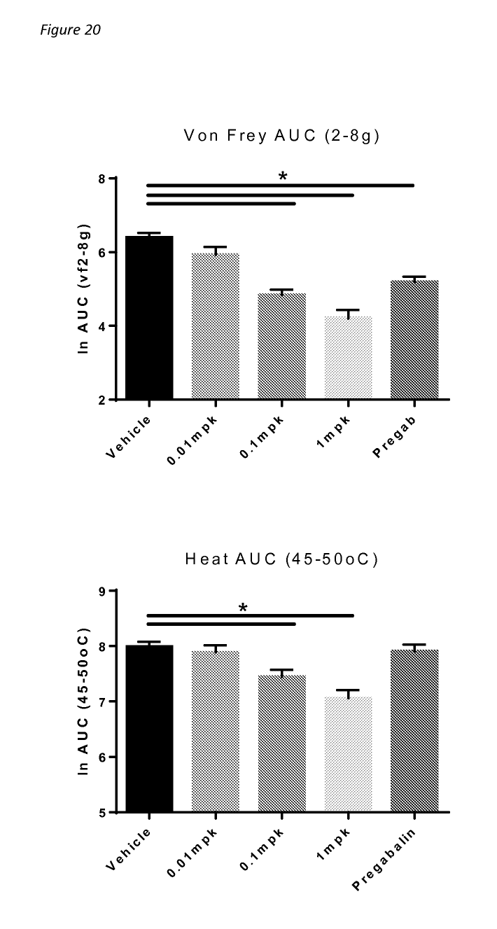

The data demonstrates that B30 dose dependently reverses measures of neuronal sensitisation associated with injury (FIG. 20). The exaggerated response profiles of spinal neurones to evoked stimuli were attenuated such that neuronal excitability was restored to pre-injury levels. Pregabalin, dosed chronically for 5 days (15 mg/kg), similarly reversed signs of neuronal hyperexcitability to mechanical punctate stimuli.

These data demonstrate the role of peripheral BDNF in maintaining sensitisation of spinal neurones following nerve injury conditions. Sequestration of BDNF by B30 may have potential utility in attenuating the pathophysiological mechanisms that drive pain such as chronic pain and neuropathic pain.

### Sequences

The following sequences are hereby disclosed as pertaining to the disclosed aspects of the present invention:

Human BDNF Amino Acid Sequence
SEQ ID NO: 1
[NCBI Reference Sequence: NP_001137277.1; 247 amino acids]
MTILFLTMVI SYFGCMKAAP MKEANIRGQG GLAYPGVRTH GTLESVNGPK AGSRGLTSLA
DTFEHVIEEL LDEDQKVRPN EENNKDADLY TSRVMLSSQV PLEPPLLFLL EEYKNYLDAA
NMSMRVRRHS DPARRGELSV CDSISEWVTA ADKKTAVDMS GGTVTVLEKV PVSKGQLKQY
FYETKCNPMG YTKEGCRGID KRHWNSQCRT TQSYVRALTM DSKKRIGWRF IRIDTSCVCT
LTIKRGR
SEQ ID NO: 2 Mouse BDNF Amino Acid Sequence
[NCBI Reference Sequence: NP_001041604.1, 249 amino acids]
MTILFLTMVI SYFGCMKAAP MKEVNVHGQG NLAYPGVRTH GTLESVNGPR AGSRGLTTTS
LADTFEHVIE ELLDEDQKVR PNEENHKDAD LYTSRVMLSS QVPLEPPLLF LLEEYKNYLD
AANMSMRVRR HSDPARRGEL SVCDSISEWV TAADKKTAVD MSGGTVTVLE KVPVSKGQLK
QYFYETKCNP MGYTKEGCRG IDKRHWNSQC RTTQSYVRAL TMDSKKRIGW RFIRIDTSCV
CTLTIKRGR
R3BH1 Chicken Clone Heavy Chain V-gene Nucleotide Sequence
SEQ ID NO: 3
gccgtgacgttggacgagtccgggggcggcctccagacgcccggaggagggctcagcctcgtctg
caaggcctccgggttcgacttcagcagttacgacatgcactgggtgcgacaggcgcccggcaaag
ggctggaatgggtcgctggtattgatgatggcggtagtgacacatactacgggtcggcggtgaag
ggccgtgccaccatctcgagggacaacgggcagagcacagtgaggctgcagctgaacaacctcag
ggctgaggacaccggcacctactactgcgccaaaagcagttatgacattagttggaatggtcatg
ttgaaaatatcgacgcatggggccacgggaccgaagtcatcgtctcctct
R3BH1 Chicken Clone Heavy Chain V-gene Amino Acid Sequence-CDRs
Underlined
SEQ ID NO: 4
AVTLDESGGGLQTPGGGLSLVCKASGFDFSSYDMHWVRQAPGKGLEWVAGIDDGGSDTYYGSAVK
GRATISRDNGQSTVRLQLNNLRAEDTGTYYCAKSSYDISWNGHVENIDAWGHGTEVIVSS
R3BH1 Chicken Clone Light Chain V-gene Nucleotide Sequence
SEQ ID NO: 5
gccctgactcagccgacctcggtgtcaacaaacctgggaggaaccgtcgagatcacctgctccgg
ggctggaagtggctatggttatggctggttccagcagaagtctcctggcagtgcccctgtcactg
tgatctatagcaacgacaagagaccctcggacatcccttcacgattctccggttctaaatccggc
tccacgggcacattaaccatcactggggtccaagccgaggacgaggctgtctatttctgtgggac
ctacgacagcactgatgctggttatgctatatttggggccgggacaaccctgaccgtccta
R3BH1 Chicken Clone Light Chain V-gene Amino Acid Sequence-CDRs
Underlined
SEQ ID NO: 6
ALTQPTSVSTNLGGTVEITCSGAGSGYGYGWFQQKSPGSAPVTVIYSNDKRPSDIPSRFSGSKSG
STGTLTITGVQAEDEAVYFCGTYDSTDAGYAIFGAGTTLTVL
R3B-H1 CDRH1
SEQ ID NO: 7
SSYDMH [Kabat]
R3B-H1 CDRH2
SEQ ID NO: 8
GIDDGGSDTYYGSAVKG [Kabat]
R3B-H1 CDRH3
SEQ ID NO: 9
SSYDISWNGHVENIDA [Kabat]
R3B-H1 CDRL1
SEQ ID NO: 10
SGAGSGYGYG [Kabat]
R3B-H1 CDRL2
SEQ ID NO: 11
SNDKRPS [Kabat]
R3B-H1 CDRL3
SEQ ID NO: 12
GTYDSTDAGYAI [Kabat]
B30 Humanised Heavy Chain V-gene Nucleotide Sequence
SEQ ID NO: 13
gaggtgcagctgttggagtctgggggaggcttggtgcagcctggggggtccctgagactctcctg
tgcagcctctgggttcgacttcagcagttacgacatgcactgggtccgccaggctccagggaagg
ggctggagtgggtctcaggtattggtgattacggtattgaaacatactacgggtccgctgtgaag
ggccggttcaccatctccagagacaattccaagaacacactgtatctgcaaatgaacagcctgag
agccgaggacaccgccgtgtattactgtgccaaaagcagttatgacattagttggaatggtcatg
ttgaacatatcgactcatggggccaggggaccctggtcaccgtctcctct
B30 Humanised Heavy Chain V-gene Amino Acid Sequence-CDRs
Underlined
SEQ ID NO: 14
EVQLLESGGGLVQPGGSLRLSCAASGFDFSSYDMHWVRQAPGKGLEWVSGIGDYGIETYYGSAVK
GRFTISRDNSKNTLYLQMNSLRAEDTAVYYCAKSSYDISWNGHVEHIDSWGQGTLVTVSS
B30 Humanised Light Chain V-gene Nucleotide Sequence
SEQ ID NO: 15
TCTTCTGAGCTGACTCAGCCTCCTGCTGTGTCTGTGGCCTTGGGACAGACAGTCAGGATCACATG
CTCCGGGGCTGGAAGTGGCTATGGTTATGGCTGGTACCAGCAGAAGCCAGGACAGGCCCCTGTGA
CCGTCATCTATAGCAACGACAAGAGACCCTCCGGGATCCCAGACCGATTCTCTGGCTCCAGCTCA
GGAAACACAGCTTCCTTGACCATCACTGGGGCTCAGGCCGAAGATGAGGCTGACTATTACTGTGG
GACCTACGTCAGCGCATATTATGGTTATGCTATATTTGGGGGCGGGACAAAGCTGACCGTCCTA
B30 Humanised Light Chain V-gene Amino Acid Sequence-CDRs
Underlined
SEQ ID NO: 16
SSELTQPPAVSVALGQTVRITCSGAGSGYGYGWYQQKPGQAPVTVIYSNDKRPSGIPDRFSGSSS
GNTASLTITGAQAEDEADYYCGTYVSAYYGYAIFGGGTKLTVL
B20 Humanised Heavy Chain V-gene Nucleotide Sequence
SEQ ID NO: 17
gaggtgcagctgttggagtctgggggaggcttggtgcagcctggggggtccctgagactctcctg
tgcagcctctgggttcgacttcagcagttacgacatgcactgggtccgccaggctccagggaagg
ggctggagtgggtctcaggtattgatgattacggaattgaaacatactacgggtccgctgtgaag
ggccggttcaccatctccagagacaattccaagaacacactgtatctgcaaatgaacagcctgag
agccgaggacaccgccgtgtattactgtgccaaaagcagttatgacattagttggaatggtcacg
tcgaacatctcgacgcatggggccaggggaccctggtcaccgtctcctct
B20 Humanised Heavy Chain V-gene Amino Acid Sequence-CDRs
Underlined
SEQ ID NO: 18
EVQLLESGGGLVQPGGSLRLSCAASGFDFSSYDMHWVRQAPGKGLEWVSGIDDYGIETYYGSAVK
GRFTISRDNSKNTLYLQMNSLRAEDTAVYYCAKSSYDISWNGHVEHLDAWGQGTLVTVSS
B20 Humanised Light Chain V-gene Nucleotide Sequence
SEQ ID NO: 19
TCTTCTGAGCTGACTCAGCCTCCTGCTGTGTCTGTGGCCTTGGGACAGACAGTCAGGATCACATG
CTCCGGGGCTGGAAGTGGCTATGGTTATGGCTGGTACCAGCAGAAGCCAGGACAGGCCCCTGTGA
CCGTCATCTATAGCAACGACAAGAGACCCTCCGGGATCCCAGACCGATTCTCTGGCTCCAGCTCA
GGAAACACAGCTTCCTTGACCATCACTGGGGCTCAGGCCGAAGATGAGGCTGACTATTACTGTGG
GACCTACGACAGCACTGATGCTGGTTATGCTATATTTGGGGGCGGGACAAAGCTGACCGTCCTA
B20 Humanised Light Chain V-gene Amino Acid Sequence-CDRs
Underlined
SEQ ID NO: 20
SSELTQPPAVSVALGQTVRITCSGAGSGYGYGWYQQKPGQAPVTVIYSNDKRPSGIPDRFSGSSS
GNTASLTITGAQAEDEADYYCGTYDSTDAGYAIFGGGTKLTVL
B18 Humanised Heavy Chain V-gene Nucleotide Sequence
SEQ ID NO: 21
gaggtgcagctgttggagtctgggggaggcttggtgcagcctggggggtccctgagactctcctg
tgcagcctctgggttcgacttcagcagttacgacatgcactgggtccgccaggctccagggaagg
ggctggagtgggtctcaggtattgatgattacggaattgaaacatactacgggtccgctgtgaag
ggccggttcaccatctccagagacaattccaagaacacactgtatctgcaaatgaacagcctgag
agccgaggacaccgccgtgtattactgtgccaaaagcagttatgacattagttggaatggtcacg
tcgaacatctcgacgcatggggccaggggaccctggtcaccgtctcctct
B18 Humanised Heavy Chain V-gene Amino Acid Sequence-CDRs
Underlined
SEQ ID NO: 22
EVQLLESGGGLVQPGGSLRLSCAASGFDFSSYDMHWVRQAPGKGLEWVSGIDDYGIETYYGSAVK
GRFTISRDNSKNTLYLQMNSLRAEDTAVYYCAKSSYDISWNGHVEHLDAWGQGTLVTVSS
B18 Humanised Light Chain V-gene Nucleotide Sequence
SEQ ID NO: 23
TCTTCTGAGCTGACTCAGCCTCCTGCTGTGTCTGTGGCCTTGGGACAGACAGTCAGGATCACATG
CCAGGGTGACAGCTCAGGATACGGTTATGGATGGTACCAGCAGAAGCCAGGACAGGCCCCTGTGA
CCGTCATCTATGGCAAGAACAATCGTCCGAGCGGGATCCCAGACCGATTCTCTGGCTCCAGCTCA
GGAAACACAGCTTCCTTGACCATCACTGGGGCTCAGGCCGAAGATGAGGCTGACTATTACTGTGG
GACCTACGTCAGCGCATATTATGGTTATGCTATATTTGGGGGCGGGACAAAGCTGACCGTCCTA
B18 Humanised Light Chain V-gene Amino Acid Sequence-CDRs
CDRs Underlined
SEQ ID NO: 24
SSELTQPPAVSVALGQTVRITCQGDSSGYGYGWYQQKPGQAPVTVIYGKNNRPSGIPDRFSGSSS
GNTASLTITGAQAEDEADYYCGTYVSAYYGYAIFGGGTKLTVL
B30 CDRH1
SEQ ID NO: 25
SSYDMH [Kabat]
B30 CDRH2
SEQ ID NO: 26
GIGDYGIETYYGSAVK [Kabat]
B30 CDRH3
SEQ ID NO: 27
SSYDISWNGHVEHIDS [Kabat]
B30 CDRL1
SEQ ID NO: 28
SGAGSGYGYG [Kabat]
B30 CDRL2
SEQ ID NO: 29
SNDKRPS [Kabat]
B30 CDRL3
SEQ ID NO: 30
GTYVSAYYGYAI [Kabat]
B20 CDRH1
SEQ ID NO: 31
SSYDMH [Kabat]
B20 CDRH2
SEQ ID NO: 32
GIDDYGIETYYGSAVK [Kabat]
B20 CDRH3
SEQ ID NO: 33
SSYDISWNGHVEHLDA [Kabat]
B20 CDRL1
SEQ ID NO: 34
SGAGSGYGYG [Kabat]
B20 CDRL2
SEQ ID NO: 35
SNDKRPS [Kabat]
B20 CDRL3
SEQ ID NO: 36
GTYDSTDAGYAI [Kabat]
B18 CDRH1
SEQ ID NO: 37
SSYDMH [Kabat]
B18 CDRH2
SEQ ID NO: 38
GIDDYGIETYYGSAVK [Kabat]
B18 CDRH3
SEQ ID NO: 39
SSYDISWNGHVEHLDA [Kabat]
B18 CDRL1
SEQ ID NO: 40
QGDSSGYGYG [Kabat]
B18 CDRL2
SEQ ID NO: 41
GKNNRPS [Kabat]
B18 CDRL3
SEQ ID NO: 42
GTYVSAYYGYAI [Kabat]

What is claimed:

1. An isolated anti-BDNF antibody, or an antigen-binding portion thereof, wherein the antibody:
(a) binds to human BDNF, and
(b) competes for binding to human BDNF with, and/or binds to the same epitope on human BDNF as, a reference antibody comprising:
(i) a heavy chain variable region comprising the amino acid sequence of SEQ ID NO:14 and a light chain variable region comprising the amino acid sequence of SEQ ID NO:16; or
(ii) a heavy chain variable region comprising the amino acid sequence of SEQ ID NO:4 and a light chain variable region comprising the amino acid sequence of SEQ ID NO:6; or
(iii) a heavy chain variable region comprising the amino acid sequence of SEQ ID NO:18 and a light chain variable region comprising the amino acid sequence of SEQ ID NO:20; or
(iv) a heavy chain variable region comprising the amino acid sequence of SEQ ID NO:22 and a light chain variable region comprising the amino acid sequence of SEQ ID NO:24; or

[symbol]

2. The isolated anti-BDNF antibody, or an antigen-binding portion thereof according to claim 1, wherein the antibody, competes for binding to human BDNF with and/or binds to the same epitope on human BDNF as a reference antibody comprising;
(i) a heavy chain region comprising the heavy chain variable region sequence encoded by the plasmid deposited at the ATCC and having ATCC Accession No. PTA-121201 and a light chain region comprising the light chain variable region sequence encoded by the plasmid deposited at the ATCC and having ATCC Accession No. PTA-121202, or
(ii) a heavy chain region comprising the heavy chain variable region sequence encoded by the plasmid deposited at the ATCC and having ATCC Accession No. PTA-121203 and a light chain region comprising the light chain variable region sequence encoded by the plasmid deposited at the ATCC and having ATCC Accession No. PTA-121204.

3. The anti-BDNF antibody, or an antigen-binding portion thereof, according to claim 1, wherein the antibody or antigen-binding portion selectively binds to human BDNF and does not bind and/or specifically bind to related neurotrophins Nerve Growth Factor (NGF), Neurotrophin-3 (NT-3), P75, and Neurotrophin-4 (NT-4).

4. The anti-BDNF antibody, or an antigen-binding portion thereof, according to claim 1, wherein the antibody or antigen-binding portion specifically binds to human BDNF with a K_{D} of less than 55 nM, optionally as measured by SPR.

5. The anti-BDNF antibody, or an antigen-binding portion thereof, according to claim 1, wherein the antibody or antigen-binding portion thereof, inhibits the binding of BDNF to the receptor TrkB and/or p75NTR.

6. The anti-BDNF antibody, or an antigen-binding portion thereof, according to claim 1, wherein the antibody, or antigen-binding portion, inhibits the binding of BDNF to the receptor TrkB and/or p75NTR with an IC50 of less than 0.5 nM.

7. The anti-BDNF antibody, or an antigen-binding portion thereof, according to claim 1, wherein the antibody, or antigen-binding portion thereof, inhibits BDNF activity and/or activation of BDNF receptor signalling pathways.

8. The anti-BDNF antibody, or an antigen-binding portion thereof, according to claim 7, wherein the antibody, or antigen-binding portion thereof, inhibits BDNF activity with an IC50 of less than 300 nM.

9. The antibody or an antigen-binding portion thereof, according to claim 1, which is human, humanised or chimeric.

10. The antibody or an antigen-binding portion thereof, according to claim 1, wherein the antibody has an isotype subclass selected from the group consisting of IgG1, of IgG₂, IgG₄, IgG_{2Δa}, IgG_{4Δb}, IgG_{4Δc}, IgG₄ S228P, IgG_{4Δb} S228P and IgG_{4Δc} S228P.

11. The antibody or an antigen binding portion thereof, according to claim 1, wherein the antibody further comprises an immunologically inert constant region.

12. The antibody or an antigen-binding portion thereof, according to claim 1, which is a single chain antibody, a Fab fragment, a F(ab)₂ fragment, a Fv fragment, a tetrameric antibody, a tetravalent antibody, a multispecific antibody, a domain-specific antibody, a single domain antibody, or a fusion protein.

13. The antibody, or antigen-binding portion thereof, according to claim 1, wherein the antibody, or antigen-binding portion thereof, binds to an epitope comprised within both BDNF monomers of the same BDNF homodimer

[symbol]

14. The antibody, or antigen-binding portion thereof, according to claim 13, wherein the antibody, or antigen-binding portion thereof, binds to an epitope comprising a region comprising loop 1 and loop 4 of a first BDNF monomer and loop 2, loop 3 and the N-terminal region of a second BDNF monomer in the BDNF homodimer.

15. The antibody, or antigen-binding portion thereof, according to claim 1, wherein the antibody binds to the epitope on human BDNF comprising residues within the region of ILE 16 to PHE 102, ILE 16 to Arg 104 or residues ILE 16 to ASN 106 of SEQ ID NO:1.

16. The antibody, or antigen-binding portion thereof, according to claim 1, wherein the epitope comprises;
(a) residues ILE 16, SER 17 TRP 19, THR 21, ALA 23, MET 31, SER 32, GLU 40, LYS 41, LYS 46, LEU 49, LYS 50, TYR 52, MET 61, ARG 88, LYS 95, ARG 97, GLY 99, TRP 100, ARG 101, PHE 102 of SEQ ID NO:1, or
(b) residues ILE 16, SER 17, TRP 19, THR 21, ALA 23, MET 31, SER 32, GLY 33, GLU 40, LYS 41, VAL 44, SER 45, GLN 48, LEU 49, LYS 50, TYR 52, TYR 86, TRP 100, ARG 101, PHE 102, ARG 104 of SEQ ID NO:1, or
(c) residues ILE 16, SER 17 TRP 19, ALA 23, MET 31, SER 32, GLU 40, LYS 41, LEU 49, LYS 50, TYR 52, MET 61, ARG 88, ARG 97, GLY 99, TRP 100, ARG 101, PHE 102 of SEQ ID NO:1, or
(d) residues ILEU 16, SER 17 TRP 19, THR 21, ALA 23, MET 31, SER 32, GLU 40, LYS 41, LYS 50, TYR 52, TRP 100, ARG 101, PHE 102 of SEQ ID NO:1
(e) residues TRP 19, LYS 41, LYS 50, TYR 52, ARG 88, ARG 97, ARG 101 of SEQ ID NO:1, or
(f) residues ILE 16, MET 31, LEU 49, GLY 99, PHE 102 of SEQ ID NO:1, or
(g) residues, THR 21, SER 32, SER 17, GLU 40, MET 61, ASP 30 of SEQ ID NO:1, or residues ALA 23, GLN 48, TRP 100 of SEQ ID NO:1, or residues ILEU 98, GLU 18, ASP 24, ARG 104 of SEQ ID NO:1, or residues THR 21, LYS 46, LYS 95, of SEQ ID NO:1.

17. The antibody, or antigen-binding portion thereof, of claim 1, which comprises:
(i) a heavy chain variable region comprising CDR1, CDR2, CDR3 of the heavy chain variable region sequence of SEQ ID NO: 14 and a light chain variable region comprising CDR1, CDR2, CDR3 of the light chain variable region sequence of SEQ ID NO: 16, or
(ii) a heavy chain variable region comprising CDR1, CDR2, CDR3 of the heavy chain variable region sequence of SEQ ID NO: 4 and a light chain variable region comprising CDR1, CDR2, CDR3 of the light chain variable region sequence of SEQ ID NO: 6, or
(iii) a heavy chain variable region comprising CDR1, CDR2, CDR3 of the heavy chain variable region sequence of SEQ ID NO: 18 and a light chain variable region comprising CDR1, CDR2, CDR3 of the light chain variable region sequence of SEQ ID NO: 20, or
(iv) a heavy chain variable region comprising CDR1, CDR2, CDR3 of the heavy chain variable region sequence of SEQ ID NO: 22 and a light chain variable region comprising CDR1, CDR2, CDR3 of the light chain variable region sequence of SEQ ID NO: 24.

18. The antibody, or antigen-binding portion thereof, of claim 1, which comprises:
(i) a heavy chain variable region comprising
a heavy chain variable region CDR1 comprising SEQ ID NO: 25,
a heavy chain variable region CDR2 comprising SEQ ID NO: 26,
a heavy chain variable region CDR3 comprising SEQ ID NO: 27, and
a light chain variable region comprising
a light chain variable region CDR1 comprising SEQ ID NO: 28,
a light chain variable region CDR2 comprising SEQ ID NO: 29 and
a light chain variable region CDR3 comprising SEQ ID NO: 30; or
(ii) a heavy chain variable region comprising;
a heavy chain variable region CDR1 comprising SEQ ID NO: 7,
a heavy chain variable region CDR2 comprising SEQ ID NO: 8,
a heavy chain variable region CDR3 comprising SEQ ID NO: 9, and
a light chain variable region comprising
a light chain variable region CDR1 comprising SEQ ID NO: 10,
a light chain variable region CDR2 comprising SEQ ID NO: 11 and
a light chain variable region CDR3 comprising SEQ ID NO: 12; or
(iii) a heavy chain variable region comprising
a heavy chain variable region CDR1 comprising SEQ ID NO: 31,
a heavy chain variable region CDR2 comprising SEQ ID NO: 32,
a heavy chain variable region CDR3 comprising SEQ ID NO: 33, and
a light chain variable region comprising
a light chain variable region CDR1 comprising SEQ ID NO: 34,
a light chain variable region CDR2 comprising SEQ ID NO: 35 and
a light chain variable region CDR3 comprising SEQ ID NO: 36; or
(iv) a heavy chain variable region comprising
a heavy chain variable region CDR1 comprising SEQ ID NO: 37,
a heavy chain variable region CDR2 comprising SEQ ID NO: 38,
a heavy chain variable region CDR3 comprising SEQ ID NO: 39, and
a light chain variable region comprising
a light chain variable region CDR1 comprising SEQ ID NO: 40,
a light chain variable region CDR2 comprising SEQ ID NO: 41 and
a light chain variable region CDR3 comprising SEQ ID NO: 42.

19. The antibody, or antigen-binding portion thereof, of claim 1, which comprises:
(i) a heavy chain region comprising the heavy chain variable region sequence of SEQ ID NO: 14 and a light chain region comprising the light chain variable region sequence of SEQ ID NO: 16, or
(ii) a heavy chain region comprising the heavy chain variable region sequence of SEQ ID NO: 4 and a light chain region comprising the light chain variable region sequence of SEQ ID NO: 6, or
(iii) a heavy chain region comprising the heavy chain variable region sequence of SEQ ID NO: 18 and a light chain region comprising the light chain variable region sequence of SEQ ID NO: 20, or
(iv) a heavy chain region comprising the heavy chain variable region sequence of SEQ ID NO: 22 and a light chain region comprising the light chain variable region sequence of SEQ ID NO: 24.

20. The antibody, or antigen-binding portion thereof, of claim 1, which comprises:
(i) a heavy chain region comprising the heavy chain variable region sequence encoded by the plasmid deposited at the ATCC and having ATCC Accession No. PTA-121201 and a light chain region comprising the light chain variable region sequence encoded by the plasmid deposited at the ATCC and having ATCC Accession No. PTA-121202, or
(ii) a heavy chain region comprising the heavy chain variable region sequence encoded by the plasmid deposited at the ATCC and having ATCC Accession No. PTA-121203 and a light chain region comprising the light chain variable region sequence encoded by the plasmid deposited at the ATCC and having ATCC Accession No. PTA-121204.

21. An immunoconjugate comprising the antibody, or antigen-binding portion thereof, of claim 1, linked to a therapeutic agent.

22. A pharmaceutical composition comprising the antibody, or antigen-binding portion thereof, of claim 1 and a pharmaceutically acceptable carrier.

23. A pharmaceutical composition comprising the immunoconjugate of claim 21 and a pharmaceutically acceptable carrier.

24. An isolated nucleic acid molecule encoding the antibody, or antigen-binding portion thereof, of claim 1.

25. An isolated nucleic acid molecule according to claim 24 encoding:
(i) a heavy chain region comprising the heavy chain variable region sequence of SEQ ID NO: 4 and/or a light chain region comprising the light chain variable region sequence of SEQ ID NO: 6, or
(ii) a heavy chain region comprising the heavy chain variable region sequence of SEQ ID NO: 14 and/or a light chain region comprising the light chain variable region sequence of SEQ ID NO: 16, or
(iii) a heavy chain region comprising the heavy chain variable region sequence of SEQ ID NO: 18 and/or a light chain region comprising the light chain variable region sequence of SEQ ID NO: 20, or
(iv) a heavy chain region comprising the heavy chain variable region sequence of SEQ ID NO: 22 and/or a light chain region comprising the light chain variable region sequence of SEQ ID NO: 24.

26. An isolated nucleic acid molecule according to claim 24 comprising a nucleic acid sequence selected from:
(i) SEQ ID NO: 3 and/or SEQ ID NO: 5,
(ii) SEQ ID NO: 13 and/or SEQ ID NO: 15,
(iii) SEQ ID NO: 17 and/or SEQ ID NO: 19, or
(iv) SEQ ID NO: 21 and/or SEQ ID NO: 23.

27. A vector comprising the nucleic acid molecule of claim 24.

28. An isolated host cell comprising the vector of claim 27.

29. A method of producing an anti-BDNF antibody, comprising culturing the host cell of claim 28 under conditions that result in expression and/or production of the antibody, and isolating the antibody from the host cell or culture.

30. A method for treating and/or preventing pain in a subject, comprising administering an effective amount of the antibody, or antigen-binding portion thereof, of claim 1.

31. A method for treating and/or preventing pain in a subject according to claim 30, wherein the pain is selected from inflammatory pain, nociceptive pain or neuropathic pain.

32. The method for treating and/or preventing pain in a subject according to claim 30, wherein the pain is chronic pain.

33. The method for treating and/or preventing pain in a subject according to claim 30, wherein the antibody, or antigen-binding portion thereof, is for separate, sequential or simultaneous use in a combination combined with a second therapeutic agent.

34. The method for treating and/or preventing pain in a subject according to claim 33 wherein the second therapeutic agent is selected from: an opioid analgesic, a nonsteroidal antiinflammatory drug (NSAID), a barbiturate sedative, a benzodiazepine having a sedative action, a sedative such as glutethimide, meprobamate, methaqualone or dichloralphenazone; a skeletal muscle relaxant, an NMDA receptor antagonist, an alpha-adrenergic, a tricyclic antidepressant, an anticonvulsant, a tachykinin (NK) antagonist, a muscarinic antagonist, a COX-2 selective inhibitor, a coal-tar analgesic, a neuroleptic; a vanilloid receptor agonist or antagonist, a beta-adrenergic; a local anaesthetic; a corticosteroid, a 5-HT receptor agonist or antagonist, a 5-HT_{2A} receptor antagonist, a cholinergic (nicotinic) analgesic, Tramadol®; a PDEV inhibitor, a cannabinoid; metabotropic glutamate subtype 1 receptor (mGluR1) antagonist; a serotonin reuptake inhibitor, a noradrenaline (norepinephrine) reuptake inhibitor, a dual serotonin-noradrenaline reuptake inhibitor, an inducible nitric oxide synthase (iNOS) inhibitor, an acetylcholinesterase inhibitor; a prostaglandin E₂ subtype 4 (EP4) antagonist, a leukotriene B4 antagonist; a 5-lipoxygenase inhibitor, a sodium channel blocker, or a 5-HT3 antagonist; and the pharmaceutically acceptable salts and solvates thereof.

35. A method for treating and/or preventing pain in a subject, comprising administering an effective amount of the immunoconjugate of claim 21.

36. The method for treating and/or preventing pain in a subject according to claim 35, wherein the pain is selected from inflammatory pain, nociceptive pain or neuropathic pain.

37. The method for treating and/or preventing pain in a subject according to claim 35, wherein the pain is chronic pain.

38. The method for treating and/or preventing pain in a subject according to claim 35, wherein the immunoconjugate is for separate, sequential or simultaneous use in a combination combined with a second therapeutic agent.

39. The method for treating and/or preventing pain in a subject according to claim 38, wherein the second therapeutic agent is selected from: an opioid analgesic, a nonsteroidal antiinflammatory drug (NSAID), a barbiturate sedative, a benzodiazepine having a sedative action, a sedative such as glutethimide, meprobamate, methaqualone or dichloralphenazone; a skeletal muscle relaxant, an NMDA receptor antagonist, an alpha-adrenergic, a tricyclic antidepressant, an anticonvulsant, a tachykinin (NK) antagonist, a muscarinic antagonist, a COX-2 selective inhibitor, a coal-tar analgesic, a neuroleptic; a vanilloid receptor agonist or antagonist, a beta-adrenergic; a local anaesthetic; a corticosteroid, a 5-HT receptor agonist or antagonist, a 5-HT_{2A} receptor antagonist, a cholinergic (nicotinic) analgesic, Tramadol®; a PDEV inhibitor, a cannabinoid; metabotropic glutamate subtype 1 receptor (mGluR1) antagonist; a serotonin reuptake inhibitor, a noradrenaline (norepinephrine) reuptake inhibitor, a dual serotonin-noradrenaline reuptake inhibitor, an inducible nitric oxide synthase (iNOS) inhibitor, an acetylcholinesterase inhibitor; a prostaglandin E₂ subtype 4 (EP4) antagonist, a leukotriene B4 antagonist; a 5-lipoxygenase inhibitor, a sodium channel blocker, or a 5-HT3 antagonist; and the pharmaceutically acceptable salts and solvates thereof.

40. A method for treating and/or preventing pain in a subject, comprising administering an effective amount of the pharmaceutical composition of claim 22.

41. The method for treating and/or preventing pain in a subject according to claim 40, wherein the pain is selected from inflammatory pain, nociceptive pain or neuropathic pain.

42. The method for treating and/or preventing pain in a subject according to claim 40, wherein the pain is chronic pain.

43. The method for treating and/or preventing pain in a subject according to claim 40, wherein the pharmaceutical composition is for separate, sequential or simultaneous use in a combination combined with a second therapeutic agent.

44. The method for treating and/or preventing pain in a subject according to claim 43, wherein the second therapeutic agent is selected from: an opioid analgesic, a nonsteroidal antiinflammatory drug (NSAID), a barbiturate sedative, a benzodiazepine having a sedative action, a sedative such as glutethimide, meprobamate, methaqualone or dichloralphenazone; a skeletal muscle relaxant, an NMDA receptor antagonist, an alpha-adrenergic, a tricyclic antidepressant, an anticonvulsant, a tachykinin (NK) antagonist, a muscarinic antagonist, a COX-2 selective inhibitor, a coal-tar analgesic, a neuroleptic; a vanilloid receptor agonist or antagonist, a beta-adrenergic; a local anaesthetic; a corticosteroid, a 5-HT receptor agonist or antagonist, a 5-HT_{2A} receptor antagonist, a cholinergic (nicotinic) analgesic, Tramadol®; a PDEV inhibitor, a cannabinoid; metabotropic glutamate subtype 1 receptor (mGluR1) antagonist; a serotonin reuptake inhibitor, a noradrenaline (norepinephrine) reuptake inhibitor, a dual serotonin-noradrenaline reuptake inhibitor, an inducible nitric oxide synthase (iNOS) inhibitor, an acetylcholinesterase inhibitor; a prostaglandin E₂ subtype 4 (EP4) antagonist, a leukotriene B4 antagonist; a 5-lipoxygenase inhibitor, a sodium channel blocker, or a 5-HT3 antagonist; and the pharmaceutically acceptable salts and solvates thereof.

45. A method for treating and/or preventing pain in a subject, comprising administering an effective amount of the pharmaceutical composition of claim 23.

46. The method for treating and/or preventing pain in a subject according to claim 45, wherein the pain is selected from inflammatory pain, nociceptive pain or neuropathic pain.

47. The method for treating and/or preventing pain in a subject according to claim 45, wherein the pain is chronic pain.

48. The method for treating and/or preventing pain in a subject according to claim 45, wherein the pharmaceutical composition is for separate, sequential or simultaneous use in a combination combined with a second therapeutic agent.

49. The method for treating and/or preventing pain in a subject according to claim 48, wherein the second therapeutic agent is selected from: an opioid analgesic, a nonsteroidal antiinflammatory drug (NSAID), a barbiturate sedative, a benzodiazepine having a sedative action, a sedative such as glutethimide, meprobamate, methaqualone or dichloralphenazone; a skeletal muscle relaxant, an NMDA receptor antagonist, an alpha-adrenergic, a tricyclic antidepressant, an anticonvulsant, a tachykinin (NK) antagonist, a muscarinic antagonist, a COX-2 selective inhibitor, a coal-tar analgesic, a neuroleptic; a vanilloid receptor agonist or antagonist, a beta-adrenergic; a local anaesthetic; a corticosteroid, a 5-HT receptor agonist or antagonist, a 5-HT_{2A} receptor antagonist, a cholinergic (nicotinic) analgesic, Tramadol®; a PDEV inhibitor, a cannabinoid; metabotropic glutamate subtype 1 receptor (mGluR1) antagonist; a serotonin reuptake inhibitor, a noradrenaline (norepinephrine) reuptake inhibitor, a dual serotonin-noradrenaline reuptake inhibitor, an inducible nitric oxide synthase (iNOS) inhibitor, an acetylcholinesterase inhibitor; a prostaglandin E₂ subtype 4 (EP4) antagonist, a leukotriene B4 antagonist; a 5-lipoxygenase inhibitor, a sodium channel blocker, or a 5-HT3 antagonist; and the pharmaceutically acceptable salts and solvates thereof.


SEQUENCE LISTING (42 sequences)

SEQ ID NO: 1 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 2 - protein, 249 aa, Mus musculus
MTILFLTMVISYFGCMKAAPMKEVNVHGQGNLAYPGVRTHGTLESVNGPRAGSRGLTTTSLADTFEHVIEELLDEDQKVRPNEENHKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 3 - DNA, 375 bp, Artificial Sequence
  R3BH1 VH DNA Seq - Chicken, human chimeric antibody
gccgtgacgttggacgagtccgggggcggcctccagacgcccggaggagggctcagcctcgtctgcaaggcctccgggttcgacttcagcagttacgacatgcactgggtgcgacaggcgcccggcaaagggctggaatgggtcgctggtattgatgatggcggtagtgacacatactacgggtcggcggtgaagggccgtgccaccatctcgagggacaacgggcagagcacagtgaggctgcagctgaacaacctcagggctgaggacaccggcacctactactgcgccaaaagcagttatgacattagttggaatggtcatgttgaaaatatcgacgcatggggccacgggaccgaagtcatcgtctcctct

SEQ ID NO: 4 - protein, 125 aa, Artificial Sequence
  R3BH1 VH Protein Seq - Chicken, human chimeric antibody
AVTLDESGGGLQTPGGGLSLVCKASGFDFSSYDMHWVRQAPGKGLEWVAGIDDGGSDTYYGSAVKGRATISRDNGQSTVRLQLNNLRAEDTGTYYCAKSSYDISWNGHVENIDAWGHGTEVIVSS

SEQ ID NO: 5 - DNA, 321 bp, Artificial Sequence
  R3BH1 VL DNA Seq  - Chicken, human chimeric antibody
gccctgactcagccgacctcggtgtcaacaaacctgggaggaaccgtcgagatcacctgctccggggctggaagtggctatggttatggctggttccagcagaagtctcctggcagtgcccctgtcactgtgatctatagcaacgacaagagaccctcggacatcccttcacgattctccggttctaaatccggctccacgggcacattaaccatcactggggtccaagccgaggacgaggctgtctatttctgtgggacctacgacagcactgatgctggttatgctatatttggggccgggacaaccctgaccgtccta

SEQ ID NO: 6 - protein, 107 aa, Artificial Sequence
  R3BH1 VL Protein Seq - Chicken, human chimeric antibody
ALTQPTSVSTNLGGTVEITCSGAGSGYGYGWFQQKSPGSAPVTVIYSNDKRPSDIPSRFSGSKSGSTGTLTITGVQAEDEAVYFCGTYDSTDAGYAIFGAGTTLTVL

SEQ ID NO: 7 - protein, 6 aa, Artificial Sequence
  R3BH1 CDRH1 Protein Seq - Chicken, human chimeric antibody
SSYDMH

SEQ ID NO: 8 - protein, 17 aa, Artificial Sequence
  R3BH1 CDRH2 Protein Seq - Chicken, human chimeric antibody
GIDDGGSDTYYGSAVKG

SEQ ID NO: 9 - protein, 16 aa, Artificial Sequence
  R3BH1 CDRH3 Protein Seq - Chicken, human chimeric antibody
SSYDISWNGHVENIDA

SEQ ID NO: 10 - protein, 10 aa, Artificial Sequence
  R3BH1 CDRL1 Protein Seq - Chicken, human chimeric antibody
SGAGSGYGYG

SEQ ID NO: 11 - protein, 7 aa, Artificial Sequence
  R3BH1 CDRL2 Protein Seq - Chicken, human chimeric antibody
SNDKRPS

SEQ ID NO: 12 - protein, 12 aa, Artificial Sequence
  R3BH1 CDRL3 Protein Seq - Chicken, human chimeric antibody
GTYDSTDAGYAI

SEQ ID NO: 13 - DNA, 375 bp, Artificial Sequence
  B30 VH DNA Seq - Humanized antibody,  chicken R3BH1 CDRs
gaggtgcagctgttggagtctgggggaggcttggtgcagcctggggggtccctgagactctcctgtgcagcctctgggttcgacttcagcagttacgacatgcactgggtccgccaggctccagggaaggggctggagtgggtctcaggtattggtgattacggtattgaaacatactacgggtccgctgtgaagggccggttcaccatctccagagacaattccaagaacacactgtatctgcaaatgaacagcctgagagccgaggacaccgccgtgtattactgtgccaaaagcagttatgacattagttggaatggtcatgttgaacatatcgactcatggggccaggggaccctggtcaccgtctcctct

SEQ ID NO: 14 - protein, 125 aa, Artificial Sequence
  B30 VH Protein Seq - Humanized antibody, chicken R3BH1 CDRs
EVQLLESGGGLVQPGGSLRLSCAASGFDFSSYDMHWVRQAPGKGLEWVSGIGDYGIETYYGSAVKGRFTISRDNSKNTLYLQMNSLRAEDTAVYYCAKSSYDISWNGHVEHIDSWGQGTLVTVSS

SEQ ID NO: 15 - DNA, 324 bp, Artificial Sequence
  B30 VL DNA Seq - Humanized antibody,  chicken R3BH1 CDRs
tcttctgagctgactcagcctcctgctgtgtctgtggccttgggacagacagtcaggatcacatgctccggggctggaagtggctatggttatggctggtaccagcagaagccaggacaggcccctgtgaccgtcatctatagcaacgacaagagaccctccgggatcccagaccgattctctggctccagctcaggaaacacagcttccttgaccatcactggggctcaggccgaagatgaggctgactattactgtgggacctacgtcagcgcatattatggttatgctatatttgggggcgggacaaagctgaccgtccta

SEQ ID NO: 16 - protein, 108 aa, Artificial Sequence
  B30 VL Protein Seq - Humanized antibody, chicken R3BH1 CDRs
SSELTQPPAVSVALGQTVRITCSGAGSGYGYGWYQQKPGQAPVTVIYSNDKRPSGIPDRFSGSSSGNTASLTITGAQAEDEADYYCGTYVSAYYGYAIFGGGTKLTVL

SEQ ID NO: 17 - DNA, 375 bp, Artificial Sequence
  B20 VH DNA Seq - Humanized antibody,  chicken R3BH1 CDRs
gaggtgcagctgttggagtctgggggaggcttggtgcagcctggggggtccctgagactctcctgtgcagcctctgggttcgacttcagcagttacgacatgcactgggtccgccaggctccagggaaggggctggagtgggtctcaggtattgatgattacggaattgaaacatactacgggtccgctgtgaagggccggttcaccatctccagagacaattccaagaacacactgtatctgcaaatgaacagcctgagagccgaggacaccgccgtgtattactgtgccaaaagcagttatgacattagttggaatggtcacgtcgaacatctcgacgcatggggccaggggaccctggtcaccgtctcctct

SEQ ID NO: 18 - protein, 125 aa, Artificial Sequence
  B20 VH Protein Seq - Humanized antibody, chicken R3BH1 CDRs
EVQLLESGGGLVQPGGSLRLSCAASGFDFSSYDMHWVRQAPGKGLEWVSGIDDYGIETYYGSAVKGRFTISRDNSKNTLYLQMNSLRAEDTAVYYCAKSSYDISWNGHVEHLDAWGQGTLVTVSS

SEQ ID NO: 19 - DNA, 324 bp, Artificial Sequence
  B20 VL DNA Seq - Humanized antibody,  chicken R3BH1 CDRs
tcttctgagctgactcagcctcctgctgtgtctgtggccttgggacagacagtcaggatcacatgctccggggctggaagtggctatggttatggctggtaccagcagaagccaggacaggcccctgtgaccgtcatctatagcaacgacaagagaccctccgggatcccagaccgattctctggctccagctcaggaaacacagcttccttgaccatcactggggctcaggccgaagatgaggctgactattactgtgggacctacgacagcactgatgctggttatgctatatttgggggcgggacaaagctgaccgtccta

SEQ ID NO: 20 - protein, 108 aa, Artificial Sequence
  B20 VL Protein Seq - Humanized antibody, chicken R3BH1 CDRs
SSELTQPPAVSVALGQTVRITCSGAGSGYGYGWYQQKPGQAPVTVIYSNDKRPSGIPDRFSGSSSGNTASLTITGAQAEDEADYYCGTYDSTDAGYAIFGGGTKLTVL

SEQ ID NO: 21 - DNA, 375 bp, Artificial Sequence; identical to SEQ ID NO: 17
  B18 VH DNA Seq - Humanized antibody,  chicken R3BH1 CDRs

SEQ ID NO: 22 - protein, 125 aa, Artificial Sequence; identical to SEQ ID NO: 18
  B18 VH Protein Seq - Humanized antibody, chicken R3BH1 CDRs

SEQ ID NO: 23 - DNA, 324 bp, Artificial Sequence
  B18 VL DNA Seq - Humanized antibody,  chicken R3BH1 CDRs
tcttctgagctgactcagcctcctgctgtgtctgtggccttgggacagacagtcaggatcacatgccagggtgacagctcaggatacggttatggatggtaccagcagaagccaggacaggcccctgtgaccgtcatctatggcaagaacaatcgtccgagcgggatcccagaccgattctctggctccagctcaggaaacacagcttccttgaccatcactggggctcaggccgaagatgaggctgactattactgtgggacctacgtcagcgcatattatggttatgctatatttgggggcgggacaaagctgaccgtccta

SEQ ID NO: 24 - protein, 108 aa, Artificial Sequence
  B18 VL Protein Seq - Humanized antibody, chicken R3BH1 CDRs
SSELTQPPAVSVALGQTVRITCQGDSSGYGYGWYQQKPGQAPVTVIYGKNNRPSGIPDRFSGSSSGNTASLTITGAQAEDEADYYCGTYVSAYYGYAIFGGGTKLTVL

SEQ ID NO: 25 - protein, 6 aa, Artificial Sequence
  B30 CDRH1 Protein Seq - Humanized antibody, chicken R3BH1 CDRs
SSYDMH

SEQ ID NO: 26 - protein, 16 aa, Artificial Sequence
  B30 CDRH2 Protein Seq - Humanized antibody, chicken R3BH1 CDRs
GIGDYGIETYYGSAVK

SEQ ID NO: 27 - protein, 16 aa, Artificial Sequence
  B30 CDRH3 Protein Seq - Humanized antibody, chicken R3BH1 CDRs
SSYDISWNGHVEHIDS

SEQ ID NO: 28 - protein, 10 aa, Artificial Sequence
  B30 CDRL1 Protein Seq - Humanized antibody, chicken R3BH1 CDRs
SGAGSGYGYG

SEQ ID NO: 29 - protein, 7 aa, Artificial Sequence
  B30 CDRL2 Protein Seq - Humanized antibody, chicken R3BH1 CDRs
SNDKRPS

SEQ ID NO: 30 - protein, 12 aa, Artificial Sequence
  B30 CDRL3 Protein Seq - Humanized antibody, chicken R3BH1 CDRs
GTYVSAYYGYAI

SEQ ID NO: 31 - protein, 6 aa, Artificial Sequence
  B20 CDRH1 Protein Seq - Humanized antibody, chicken R3BH1 CDRs
SSYDMH

SEQ ID NO: 32 - protein, 16 aa, Artificial Sequence
  B20 CDRH2 Protein Seq - Humanized antibody, chicken R3BH1 CDRs
GIDDYGIETYYGSAVK

SEQ ID NO: 33 - protein, 16 aa, Artificial Sequence
  B20 CDRH3 Protein Seq - Humanized antibody, chicken R3BH1 CDRs
SSYDISWNGHVEHLDA

SEQ ID NO: 34 - protein, 10 aa, Artificial Sequence
  B20 CDRL1 Protein Seq - Humanized antibody, chicken R3BH1 CDRs
SGAGSGYGYG

SEQ ID NO: 35 - protein, 7 aa, Artificial Sequence
  B20 CDRL2 Protein Seq - Humanized antibody, chicken R3BH1 CDRs
SNDKRPS

SEQ ID NO: 36 - protein, 12 aa, Artificial Sequence
  B20 CDRL3 Protein Seq - Humanized antibody, chicken R3BH1 CDRs
GTYDSTDAGYAI

SEQ ID NO: 37 - protein, 6 aa, Artificial Sequence
  B18 CDRH1 Protein Seq - Humanized antibody, chicken R3BH1 CDRs
SSYDMH

SEQ ID NO: 38 - protein, 16 aa, Artificial Sequence
  B18 CDRH2 Protein Seq - Humanized antibody, chicken R3BH1 CDRs
GIDDYGIETYYGSAVK

SEQ ID NO: 39 - protein, 16 aa, Artificial Sequence
  B18 CDRH3 Protein Seq - Humanized antibody, chicken R3BH1 CDRs
SSYDISWNGHVEHLDA

SEQ ID NO: 40 - protein, 10 aa, Artificial Sequence
  B18 CDRL1 Protein Seq - Humanized antibody, chicken R3BH1 CDRs
QGDSSGYGYG

SEQ ID NO: 41 - protein, 7 aa, Artificial Sequence
  B18 CDRL2 Protein Seq - Humanized antibody, chicken R3BH1 CDRs
GKNNRPS

SEQ ID NO: 42 - protein, 12 aa, Artificial Sequence
  B18 CDRL3 Protein Seq - Humanized antibody, chicken R3BH1 CDRs
GTYVSAYYGYAI